(19) United States
(12) Patent Application Publication
(10) Pub. No.: US 2022/0306741 A1
(43) Pub. Date: Sep. 29, 2022

(54) Purification Method for Bispecific antigen-binding Polypeptides with Enhanced Protein L Capture Dynamic Binding Capacity

(71) Applicant: AMGEN INC., Thousand Oaks, CA (US)

(72) Inventors: Ashish Sharma, Lincoln, RI (US); Balakumar Thangaraj, Lexington, MA (US)

(21) Appl. No.: 17/641,736

(22) PCT Filed: Sep. 10, 2020
(86) PCT No.: PCT/US20/50063

§ 371 Date: Mar. 9, 2022

Related U.S. Application Data
(60) Provisional application No. 62/898,495, filed on Sep. 10, 2019.

(51) Int. Cl. C07K 16/28; C07K 1/14

(52) U.S. Cl. CPC C07K 16/2809; C07K 1/145; C07K 16/2803; C07K 16/2878; C07K 2317/31; C07K 2317/622; C07K 2317/53; C07K 2317/524; C07K 2317/526

(57) ABSTRACT

The present invention provides a downstream purification method process for the production of bispecific antigen-binding polypeptides. The method comprises at least the steps of (i) providing a separation resin comprising a polymer matrix part and a ligand part, wherein the matrix part comprises polymethacrylate and has a particle size of about 30 to 60 pm, wherein the ligand part comprises recombinant protein L, and wherein the ligand part's protein L is covalently bound to the matrix part's particles, (ii) contacting a process fluid comprising the bispecific antigen-binding polypeptide with the separation resin, (iii) capturing the bispecific antigen-binding polypeptide by the ligand part of the separation resin, wherein the bispecific antigen-binding polypeptide reversibly binds to the ligand part of the separation resin, and wherein the remainder of the process fluid does not bind to the ligand part of the separation resin, (iv) washing the bound bispecific antigen-binding polypeptide with a wash buffer which does not elute the bispecific antigen-binding polypeptide from the ligand portion, and (v) elute the bispecific antigen-binding polypeptide from the ligand part with an elution buffer at a low pH.

### TECHNICAL FIELD

This invention relates to methods of biotechnology, in particular to downstream purification of bispecific antigen-binding polypeptides.

### BACKGROUND

Despite the advances in manufacturing, new protein-based pharmaceuticals require new optimized manufacturing process in order to avoid product quality impact such as protein aggregation. This affects upstream manufacturing, downstream manufacturing, storage and application.

Such new protein-based pharmaceuticals comprise, for example, bispecific antigen-binding polypeptides including (monoclonal) antibodies. A bispecific polypeptide such as an antibody is an artificial protein that can simultaneously bind to two different types of antigen. They are known in several structural formats, and current applications have been explored for cancer immunotherapy and drug delivery (Fan, Gaowei; Wang, Zujian; Hao, Mingju; Li, Jinming (2015). “Bispecific antibodies and their applications”. Journal of Hematology & Oncology. 8: 130).

In general, bispecific antibodies can be IgG-like, i.e. full length bispecific antibodies, or non-IgG-like bispecific antibodies, which are, e.g., not full-length antibodies. Full length bispecific antibodies typically retain the traditional monoclonal antibody (mAb) structure of two Fab arms and one Fc region, except the two Fab sites bind different antigens. Non full-length bispecific antibodies lack an Fc region entirely. These include chemically linked Fabs, consisting of only the Fab regions, and various types of bivalent and trivalent single-chain variable fragments (scFvs). There are also fusion proteins mimicking the variable domains of two antibodies. The likely furthest developed of these newer formats are the bi-specific T-cell engager (BiTE®) molecules (Yang, Fa; Wen, Weihong; Qin, Weijun (2016). “Bispecific Antibodies as a Development Platform for New Concepts and Treatment Strategies”. International Journal of Molecular Sciences. 18 (1): 48).

Bispecific antigen-binding polypeptides such as BiTE® molecules are recombinant protein constructs made from two flexibly linked antibody-derived binding domains. One binding domain of BiTE® molecules is specific for a selected tumor-associated surface antigen on target cells; the second binding domain is specific for CD3, a subunit of the T cell receptor complex on T cells. By their particular design BiTE® molecules are uniquely suited to transiently connect T cells with target cells and, at the same time, potently activate the inherent cytolytic potential of T cells against target cells. An important further development of the first generation of BiTE® molecules (see WO 99/54440 and WO 2005/040220) developed into the clinic as AMG 103 and AMG 110 was the provision of bispecific molecules binding to a context independent epitope at the N-terminus of the CD3ε chain (WO 2008/119567). BiTE® molecules binding to this elected epitope do not only show cross-species specificity for human and Callithrix jacchus, Saguinus oedipus or Saimiri sciureus CD3E chain, but also, due to recognizing this specific epitope instead of previously described epitopes for CD3 binders in bispecific T cell engaging molecules, do not unspecifically activate T cells to the same degree as observed for the previous generation of T cell engaging antibodies. This reduction in T cell activation was connected with less or reduced T cell redistribution in patients, which was identified as a risk for side effects.

Currently, bispecific antigen-binding polypeptides are typically processed downstream chromatographic purification employing affinity resins for antibody fragment purification. In the case of bispecific antibodies are constructs which lack the Fc region necessary for binding to Protein A, some BiTE® molecules do, alternative ligands are required for their affinity purification. Protein L, which is isolated from the surface of bacterial species, has been found to bind immunoglobulin through the light chain which bispecific antigen-binding polypeptides possess. Such affinity resins typically comprise an immunoglobulin-binding recombinant protein L ligand in a rigid, high-flow agarose matrix, wherein the ligand has a strong affinity to the variable region of antibody kappa light chains. Such resins are thought to be suitable for the capture of a wide range of antibody fragments such as fragment antibody binding (Fabs), dAbs, and single-chain fragment variable (scFv) and intend to have high binding capacity, low ligand leakage, and selectivity for a broad range of antibody fragments, thereby preferably reducing process time and amount of resin. However, new complex molecules such as bispecific antigen-binding polypeptides having a scFv format require specific and tailored downstream purification solutions in order to make full use of their potential benefits. A new bispecific antigen-binding polypeptide having advantageous therapeutic properties will not be of practical benefit if available purification methods lead to, for example, poor monomer contents, long purification time and thus, overall underwhelming productivity.

Hence, there is a need for an improved downstream purification method specifically for the production of bispecific antigen-binding polypeptides, which both increases the product quantity and the product quality in order to provide sufficient product amounts at a commercial scale at such a quality that less product needs to be discarded in downstream processing. New process methods that provide even incremental improvements in recombinant protein production and recovery are valuable, given the expense of large scale cell culture processes and the growing demand for greater quantities of and lower costs for biological products to be supplied to patients with severe unmet medical needs.

### SUMMARY

Surprisingly, an adapted downstream purification method can be provided which both ensures improved bispecific antibody product quantity and the product quality. Even if several materials for downstream antibody fragment purification are known, including different Protein L resins, it has so far not been determined which is most suited for the production of scFv bispecific antigen-binding polypeptides.

Hence, in one aspect, it is envisaged in the context of the present invention to provide A method for purifying a bispecific antigen-binding polypeptide comprising a first domain which binds to a cell surface antigen, and a second domain which binds to an extracellular epitope of the human and the Macaca CD3ε chain, wherein the method comprises the steps of

(a) providing a separation resin comprising a polymer matrix part and a ligand part, wherein the matrix part comprises a polymer, preferably polymethacrylate, and has a particle size of at least 10 μm, preferably of at least 20 μm, more preferably of about 30 to 60 μm, wherein the ligand part comprises recombinant protein L, and wherein the ligand part's protein L is covalently bound to the matrix part's particles,
(b) contacting a process fluid comprising the bispecific antigen-binding polypeptide with the separation resin,
(c) capturing the bispecific antigen-binding polypeptide by the ligand part of the separation resin, wherein the bispecific antigen-binding polypeptide reversibly binds to the ligand part of the separation resin, and wherein the remainder of the process fluid does not bind to the ligand part of the separation resin,
(c) washing the bound bispecific antigen-binding polypeptide with a wash buffer which does not elute the bispecific antigen-binding polypeptide from the ligand portion, and
(d) elute the bispecific antigen-binding polypeptide from the ligand part with an elution buffer at an acidic pH.

According to said aspect of the present invention, the matrix part has a particle size of about 45 μm.

According to said aspect of the present invention, the recombinant protein L comprises a modified B4 domain with an alkali-stable tetramer ligand having multiple coupling sites.

The method according to claim 1, wherein the recombinant protein L reversibly binds to a bispecific antigen-binding polypeptide's κ-light chain outside of the antigen binding site.

According to said aspect of the present invention, the process fluid is passed through the separation resin at least one time (purification cycle) allowing the bispecific antigen-binding polypeptide to contact with the protein L (residence time), wherein bispecific antigen-binding polypeptide residence time before elution is at least about 2 minutes, preferably about 2.5 to 4 minutes.

According to said aspect of the present invention, the wash buffer comprises at least one of the compound selected from the group consisting of phosphate buffered saline (PBS) preferably in the range of 0.01 to 1 times concentration, 3-(N-morpholino)propanesulfonic acid (MOPS) preferably in the range of 0 to 30 mM, NaCl preferably in the range of 50 to 150 mM, Tris preferably in the range 15 to 35 mM, Arginine preferably in the range 0.25 to 1 M, and Acetate preferably in the range 40-60 mM, wherein the wash puffer is in the range of pH 5 to 8.

According to said aspect of the present invention, the elution buffer comprises at least one of the compound selected from the group consisting of Tris preferably in the range of 15 to 35 mM, Arginine preferably in the range of 0.25 to 1 M, Glycine preferably in the range of 50 to 150 mM and Acetate preferably in the range of 50 to 150 mM, wherein the elution buffer has a pH in the range of about 3 to 7.5, preferably pH 3.3 to 4.2.

According to said aspect of the present invention, the dynamic loading capacity is at least 10 mg/ml resin, preferably at least 15 mg/ml resin, more preferably at least 18 mg/ml resin.

According to said aspect of the present invention, elution binding capacity is at least 7.5 mg/ml resin, preferably at least 9 mg/ml resin, more preferably 16 mg/ml resin.

According to said aspect of the present invention, further comprising a third domain which comprises two polypeptide monomers, each comprising a hinge, a CH2 domain and a CH3 domain, wherein said two polypeptide monomers are fused to each other via a peptide linker.

According to said aspect of the present invention, the antigen-binding polypeptide is a single chain antigen-binding polypeptide.

According to said aspect of the present invention, said third domain comprises in an amino to carboxyl order:

hinge-CH2-CH3-linker-hinge-CH2-CH3.

According to said aspect of the present invention, each of said polypeptide monomers in the third domain has an amino acid sequence that is at least 90% identical to a sequence selected from the group from the group consisting of: SEQ ID NO: 203-210.

According to said aspect of the present invention, each of said polypeptide monomers has an amino acid sequence selected from SEQ ID NO: 203-210.

According to said aspect of the present invention, the CH2 domain comprises an intra domain cysteine disulfide bridge.

According to said aspect of the present invention,

(i) the first domain comprises two antibody variable domains and the second domain comprises two antibody variable domains;
(ii) the first domain comprises one antibody variable domain and the second domain comprises two antibody variable domains;
(iii) the first domain comprises two antibody variable domains and the second domain comprises one antibody variable domain; or
(iv) the first domain comprises one antibody variable domain and the second domain comprises one antibody variable domain.

According to said aspect of the present invention, the first and second domain are fused to the third domain via a peptide linker.

According to said aspect of the present invention, the antigen-binding polypeptide comprises in an amino to carboxyl order:

(a) the first domain;
(b) a peptide linker preferably having an amino acid sequence selected from the group consisting of SEQ ID NOs: 187-189;
(c) the second domain.

According to said aspect of the present invention, the antigen-binding polypeptide further comprises in an amino to carboxyl order:

(d) a peptide linker having an amino acid sequence selected from the group consisting of SEQ ID NOs: 187, 188, 189, 195, 196, 197, and 198,
(e) the first polypeptide monomer of the third domain;
(f) a peptide linker having an amino acid sequence selected from the group consisting of SEQ ID NOs: 191, 192, 193 and 194; and
(g) the second polypeptide monomer of the third domain.

According to said aspect of the present invention, the first domain of the antigen-binding polypeptide binds to an epitope of CD33, CD19, BCMA, PSMA, EGFRvIII, MUC17, FLT3, CD70, DLL3, CDH3 or EpCAM, preferably CD33.

According to said aspect of the present invention, the first binding domain comprises a VH region comprising CDR-H 1, CDR-H2 and CDR-H3 selected from:

(a) CDR-H1 as depicted in SEQ ID NO: 1, CDR-H2 as depicted in SEQ ID NO: 2, CDR-H3 as depicted in SEQ ID NO: 3, CDR-L1 as depicted in SEQ ID NO: 4, CDR-L2 as depicted in SEQ ID NO: 5 and CDR-L3 as depicted in SEQ ID NO: 6,
(b) CDR-H1 as depicted in SEQ ID NO: 29, CDR-H2 as depicted in SEQ ID NO: 30, CDR-H3 as depicted in SEQ ID NO: 31, CDR-L1 as depicted in SEQ ID NO: 34, CDR-L2 as depicted in SEQ ID NO: 35 and CDR-L3 as depicted in SEQ ID NO: 36,
(c) CDR-H1 as depicted in SEQ ID NO: 42, CDR-H2 as depicted in SEQ ID NO: 43, CDR-H3 as depicted in SEQ ID NO: 44, CDR-L1 as depicted in SEQ ID NO: 45, CDR-L2 as depicted in SEQ ID NO: 46 and CDR-L3 as depicted in SEQ ID NO: 47,
(d) CDR-H1 as depicted in SEQ ID NO: 53, CDR-H2 as depicted in SEQ ID NO: 54, CDR-H3 as depicted in SEQ ID NO: 55, CDR-L1 as depicted in SEQ ID NO: 56, CDR-L2 as depicted in SEQ ID NO: 57 and CDR-L3 as depicted in SEQ ID NO: 58,
(e) CDR-H1 as depicted in SEQ ID NO: 65, CDR-H2 as depicted in SEQ ID NO: 66, CDR-H3 as depicted in SEQ ID NO: 67, CDR-L1 as depicted in SEQ ID NO: 68, CDR-L2 as depicted in SEQ ID NO: 69 and CDR-L3 as depicted in SEQ ID NO: 70,
(f) CDR-H1 as depicted in SEQ ID NO: 83, CDR-H2 as depicted in SEQ ID NO: 84, CDR-H3 as depicted in SEQ ID NO: 85, CDR-L1 as depicted in SEQ ID NO: 86, CDR-L2 as depicted in SEQ ID NO: 87 and CDR-L3 as depicted in SEQ ID NO: 88,
(g) CDR-H1 as depicted in SEQ ID NO: 94, CDR-H2 as depicted in SEQ ID NO: 95, CDR-H3 as depicted in SEQ ID NO: 96, CDR-L1 as depicted in SEQ ID NO: 97, CDR-L2 as depicted in SEQ ID NO: 98 and CDR-L3 as depicted in SEQ ID NO: 99,
(h) CDR-H1 as depicted in SEQ ID NO: 105, CDR-H2 as depicted in SEQ ID NO: 106, CDR-H3 as depicted in SEQ ID NO: 107, CDR-L1 as depicted in SEQ ID NO: 109, CDR-L2 as depicted in SEQ ID NO: 110 and CDR-L3 as depicted in SEQ ID NO: 111,
(i) CDR-H1 as depicted in SEQ ID NO: 115, CDR-H2 as depicted in SEQ ID NO: 116, CDR-H3 as depicted in SEQ ID NO: 117, CDR-L1 as depicted in SEQ ID NO: 118, CDR-L2 as depicted in SEQ ID NO: 119 and CDR-L3 as depicted in SEQ ID NO: 120,
(j) CDR-H1 as depicted in SEQ ID NO: 126, CDR-H2 as depicted in SEQ ID NO: 127, CDR-H3 as depicted in SEQ ID NO: 128, CDR-L1 as depicted in SEQ ID NO: 129, CDR-L2 as depicted in SEQ ID NO: 130 and CDR-L3 as depicted in SEQ ID NO: 131,
(k) CDR-H1 as depicted in SEQ ID NO: 137, CDR-H2 as depicted in SEQ ID NO: 138, CDR-H3 as depicted in SEQ ID NO: 139, CDR-L1 as depicted in SEQ ID NO: 140, CDR-L2 as depicted in SEQ ID NO: 141 and CDR-L3 as depicted in SEQ ID NO: 142,
(l) CDR-H1 as depicted in SEQ ID NO: 152, CDR-H2 as depicted in SEQ ID NO: 153, CDR-H3 as depicted in SEQ ID NO: 154, CDR-L1 as depicted in SEQ ID NO: 155, CDR-L2 as depicted in SEQ ID NO: 156 and CDR-L3 as depicted in SEQ ID NO: 157, and
(m) CDR-H1 as depicted in SEQ ID NO: 167, CDR-H2 as depicted in SEQ ID NO: 168, CDR-H3 as depicted in SEQ ID NO: 169, CDR-L1 as depicted in SEQ ID NO: 170, CDR-L2 as depicted in SEQ ID NO: 171 and CDR-L3 as depicted in SEQ ID NO: 172.

According to said aspect of the present invention, the antigen-binding polypeptide comprises in an amino to carboxyl order:

(a) the first domain as described above,
(b) a peptide linker having an amino acid sequence selected from the group consisting of SEQ ID NOs: 187-189;
(c) the second domain having an amino acid sequence selected from the group consisting of SEQ ID NOs: 23, 25, 41, 43, 59, 61, 77, 79, 95, 97, 113, 115, 131, 133, 149, 151, 167, 169, 185 or 187 of WO 2008/119567.

According to said aspect of the present invention, the antigen-binding polypeptide further comprises in an amino to carboxyl order:

(d) a peptide linker having an amino acid sequence selected from the group consisting of SEQ ID NOs: 187, 188, 189, 195, 196, 197, and 198,
(e) the first polypeptide monomer of the third domain having a polypeptide sequence selected from the group consisting of SEQ ID NOs: 203-210;
(f) a peptide linker having an amino acid sequence selected from the group consisting of SEQ ID NOs: 191, 192, 193, 194 and 195; and
(g) the second polypeptide monomer of the third domain having a polypeptide sequence selected from the group consisting of SEQ ID NOs: 203-210.

According to said aspect of the present invention, the bispecific antigen-binding polypeptide has an amino acid sequence selected from the group consisting of the “bispecific (HLE) molecules according to Table 11.

According to said aspect of the present invention, a pharmaceutical composition comprises the bispecific antigen-binding polypeptide described herein.

According to said aspect of the present invention, the bispecific antibody is for use in the prevention, treatment or amelioration of a disease selected from a proliferative disease, a tumorous disease, cancer or an immunological disorder.

According to a second aspect of the present invention, a method is provided for improving the yield of a production process for a bispecific antigen-binding polypeptide, wherein in downstream processing the method according to the first aspect is applied.

### DESCRIPTION OF THE FIGURES

FIG. 1 shows four chromatograms with bispecific antigen-binding polypeptide elution characteristics under four different Protein L resins in capture chromatography columns: (A) TOYOPEARL® AF-rProtein L-650F resin, (B) GE Kappaselect Protein L resin, (C) GE Lambdaselect Protein L resin, and (D) Kappa XL protein L resin. In (A), Significant elution peak post wash was achieved, while in (B), (C) and (D), no significant elution peak was observed post wash, however, load breakthrough happened unfavorably early.

FIG. 2 shows the binding capacity comparison between traditional Capto L resin [grey bars] versus TOYOPEARL® AF-rProtein L-650F [black bars] during loading and elution phases with respect to a CD33×CD3 bispecific antigen-binding polypeptide.

### DETAILED DESCRIPTION

A downstream purification method for the manufacturing therapeutic proteins, in particular scFv bispecific antigen-binding polypeptides, is herein provided. The present invention is envisaged to gear the downstream process to the specific needs of manufacturing bispecific antigen-binding polypeptides. Said downstream purification method does not only contribute to increased productivity and less requirement for space in comparison to standard purification using protein L filled columns known in the art. Even more, the present method as a chromatographic capture step within downstream processing is specifically adapted for bispecific antibodies and is envisaged to result in higher product quality, i.e. less aggregated bispecific antibodies in terms of higher monomer content with respect to using protein L filled columns such as Capto® L.

It was found that employing a specific chromatographic capture step within downstream purification according to the present invention, i.e. preferably using a recombinant protein L ligand covalently bound to a base matrix preferably of polymethacrylate having preferably a particle size of about 30 to 60 μm, results preferably in a significant enhancement in dynamic binding capacity, and leads to higher loading and reduction in harvested cell culture fluid pool volume. This overall leads to reduced facility fit.

Due to the achieved high elution binding capacity of typically more than 10 g/L packed resin, such as e.g. 12.7 g/L-packed resin with TOYOPEARL® AF-rProtein L-650F resin, a factor of at least two, preferably at least three, or even four improvement with respect to elution binding capacity is seen over the standard affinity resin Capto® L. The overall yield was in the similar range as per the current process. What was surprising in view of the state of the art was that several other benefits are achievable with such improvement in (elution) binding capacity. Employing a chromatography capture step within the downstream processing using a resin with a four factor improved (elution) binding capacity compared to Capto L such as, for example, the TOYOPEARL AF-rProtein L-650F, surprisingly leads to six factor reduction in the number of required purification cycles for a given volume of process fluid, i.e. the reduction of a given volume of harvested cell culture fluid, e.g. from about 12 purification cycles down to 2 purification cycles with respect to a CD33×CD3 bispecific antigen-binding polypeptide as described herein. As the skilled person appreciates, such a significant reduction of required purification cycles reduces the amount of time, space and energy to process a given amount of process fluid comprising the bispecific antigen-binding polypeptide to purify.

In the context of the present invention, the increased efficiency corresponds significantly to a reduction of bispecific antigen-binding polypeptide residence time on the resin within one purification cycle. Also, increased efficacy is associated with less time required to load at max binding capacity at a given residence. For example, at a residence time of 3 minutes compared to 5 minutes with conventional Capto L, the time taken to load at max binding capacity is reduced from about 7 hours to about 4 hours.

In the context of the present invention, one purification cycle corresponds to the time span of the target protein being loaded onto the resin, residing on the resin in the separation column allowing for time for washing plus the time it takes to elute the protein. The loading time typically takes several hours, however, preferably not more than 7 hours, more preferably not more than 5 hours, while the residence time can preferably be as short as 2 minutes or last, 3, 4 or 5 minutes. Longer protein residence times, and thus, purification cycles are uncommon in the context of the present invention and not preferred. For example, in order to achieve for higher load factor for, e.g. a BCMA×CD3 bispecific antigen-binding polypeptide as described herein, to target 18 g/L, loading time typically takes up to 7 hours. Typically, loading is the biggest time factor for a cycle. Hence, the cycle duration depends on the time taken to load at maximum binding capacity which is typically 80-90% of dynamic binding capacity of the respective resin.

In the context of the present invention, residence time is calculated as column bed height divided by the linear flow rate velocity. For example, if the residence time is 3 min then the load duration will be fast as protein will be spending less time in the column. Alternatively, for a 6 min residence time, the load duration is longer, as for the same bed height the linear flow rate [cm/h] is halved. Accordingly, longer residence times are not preferred in the context of the present invention. However, if the target load factor is very high, and maximum binding capacity is also high, then a longer loading time is contemplated within the context of the present invention. The present invention aims to load more bispecific antigen-binding polypeptide in short amount of processing time or at small residence time.

In the context of the present invention, one purification cycle typically involves equilibration, load, at least one washing step comprising wash 1 which is same as equilibration, and optionally wash 2, elution, strip, wash, optionally regeneration between cycles but typically only after the last cycle of the batch, and storage.

In the context of the present invention, proteins A and G are understood to bind to the Fc region in the heavy chains, while protein L binds to K-light chains outside of the antigen binding site. Structural studies show that well-defined motifs—domains E, D, A, B, and C in protein A; C1, D1, and C2 in protein G; B1, B2, B3, and B4 in protein L—are responsible for binding.

In the context of the present invention, a protein L being modified in its B4 domain is preferred such as TOYOPEARL AF-rProtein L-650F. Typically, TOYOPEARL AF-rProtein L-650F comprises a matrix comprising a polymer, preferably polymethacrylate, and has a particle size of preferably about 30 to 60 μm, to which matrix the ligand protein L being modified in its B4 domain is covalently bound to.

In the context of the present invention, target loading (g/L of packed resin) is understood as at least 80%, preferably at least 90% of dynamic binding capacity, which when executed in action on the resin, typically cycle after cycle, no early load breakthrough is observed. This is preferred in the context of the present invention. Early load breakthrough is understood herein as a phenomenon observed when the resin is no longer able to hold onto the determined setpoint load factor for the molecule to be bound to the resin, e.g. an antigen-binding polypeptide, and instead of the molecule binding to the resin it is present in the liquid passing the resin, such as the flow-through load sample. Typically, load breakthrough happens when the concentration of the loaded molecule, such as a bispecific antigen-binding polypeptide, in the flow through pool becomes the same as the feed solution concentration.

In the context of the present invention, Elution binding capacity (g/L of packed resin) is understood as the maximum amount of molecule to be purified, e.g. a bispecific antigen-binding polypeptide, that is typically recovered in the elution pool which was eluted as a result of using a buffer which has typically a higher affinity to the ligand of the resin as compared to the molecule to be purified. The elution binding capacity is also typically represented by the recovery yield percentage of the resin, typically an affinity resin, and is calculated as a percentage of the total antibody recovered (mass) which was loaded per volume of packed resin. Theoretically, elution binding capacity should be equal to load binding capacity, but typically depending on the strength of the elution buffer used, the elution binding capacity is less than the loading binding capacity, as not all protein loaded on to the resin is eluted out.

In the context of the present invention, Column ID (cm) is understood as column inner diameter. The larger the diameter, the more process fluid can pass in a given time frame.

In the context of the present invention, by “cell culture” or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells may be cultured in suspension or while attached to a solid substrate.

The term “mammalian cell” means any cell from or derived from any mammal (e.g., a human, a hamster, a mouse, a green monkey, a rat, a pig, a cow, or a rabbit). For example, a mammalian cell can be an immortalized cell. In some embodiments, the mammalian cell is a differentiated cell. In some embodiments, the mammalian cell is an undifferentiated cell. Non-limiting examples of mammalian cells are described herein. A preferred type of mammalian cells in the context of the present invention are GS-KO cells. Additional examples of mammalian cells are known in the art.

As used herein, the terms “cell culturing medium” (also called “culture medium,” “cell culture media,” “tissue culture media,”) refers to any nutrient solution used for growing cells, e.g., animal or mammalian cells, and which generally provides at least one or more components from the following: an energy source (usually in the form of a carbohydrate such as glucose); one or more of all essential amino acids, and generally the twenty basic amino acids, plus cysteine; vitamins and/or other organic compounds typically required at low concentrations; lipids or free fatty acids; and trace elements, e.g., inorganic compounds or naturally occurring elements that are typically required at very low concentrations, usually in the micromolar range.

Cell culture media include those that are typically employed in and/or are known for use with any cell culture process, such as, but not limited to, batch, extended batch, fed-batch and/or perfusion or continuous culturing of cells.

A “perfusion” cell culture medium or feed medium refers to a cell culture medium that is typically used in cell cultures that are maintained by perfusion or continuous culture methods and is sufficiently complete to support the cell culture during this process. Perfusion cell culture medium formulations may be richer or more concentrated than base cell culture medium formulations to accommodate the method used to remove the spent medium. Perfusion cell culture medium can be used during both the growth and production phases.

The term “0.5× volume” means about 50% of the volume. The term “0.6× volume” means about 60% of the volume. Likewise, 0.7×, 0.8×, 0.9×, and 1.0× means about 70%, 80%, 90%, or 100% of the volume, respectively.

The term “culturing” or “cell culturing” means the maintenance or proliferation of a mammalian cell under a controlled set of physical conditions.

The term “culture of mammalian cells” means a liquid culture medium containing a plurality of mammalian cells that is maintained or proliferated under a controlled set of physical conditions.

The term “liquid culture medium” means a fluid that contains sufficient nutrients to allow a cell (e.g., a mammalian cell) to grow or proliferate in vitro. For example, a liquid culture medium can contain one or more of: amino acids (e.g., 20 amino acids), a purine (e.g., hypoxanthine), a pyrimidine (e.g., thymidine), choline, inositol, thiamine, folic acid, biotin, calcium, niacinamide, pyridoxine, riboflavin, thymidine, cyanocobalamin, pyruvate, lipoic acid, magnesium, glucose, sodium, potassium, iron, copper, zinc, and sodium bicarbonate. In some embodiments, a liquid culture medium can contain serum from a mammal. In some embodiments, a liquid culture medium does not contain serum or another extract from a mammal (a defined liquid culture medium). In some embodiments, a liquid culture medium can contain trace metals, a mammalian growth hormone, and/or a mammalian growth factor. Another example of liquid culture medium is minimal medium (e.g., a medium containing only inorganic salts, a carbon source, and water). Non-limiting examples of liquid culture medium are described herein. Additional examples of liquid culture medium are known in the art and are commercially available. A liquid culture medium can contain any density of mammalian cells. For example, as used herein, a volume of liquid culture medium removed from a bioreactor can be substantially free of mammalian cells.

The term “continuous process” means a process which continuously feeds fluid through at least a part of the system. For example, in any of the exemplary continuous biological manufacturing systems described herein, a liquid culture medium containing a recombinant therapeutic protein is continuously fed into the system while it is in operation and a therapeutic protein drug substance is fed out of the system.

The term “clipping” means the partial cleaving of expressed protein, usually by proteolysis.

The term “degradation” generally means the disintegration of a larger entity, such as a peptide or protein, into at least two smaller entities, whereof one entity may be significantly larger than the other entity or entities.

The term “deamidation” means any a chemical reaction in which an amide functional group in the side chain of an amino acid, typically asparagine or glutamine, is removed or converted to another functional group. Typically, asparagine is converted to aspartic acid or isoaspartic acid.

The term “aggregation” generally refers to the direct mutual attraction between molecules, e.g. via van der Waals forces or chemical bonding. In particular, aggregation is understood as proteins accumulating and clumping together. Aggregates may include amorphous aggregates, oligomers, and amyloid fibrils and are typically referred to as high molecular weight (HMW) species, i.e. molecules having a higher molecular weight than pure product molecules which are non-aggregated molecules, typically referred to herein also as low molecular weight (LMW) species or monomer.

Acidic species are typically understood herein to be comprised in variants which are commonly observed when antibodies are analyzed by charged based-separation techniques such as isoelectric focusing (IEF) gel electrophoresis, capillary isoelectric focusing (cIEF) gel electrophoresis, cation exchange chromatography (CEX) and anion exchange chromatography (AEX). These variants are referred to as acidic or basic species as compared with the main species. Acidic species are typically variants with lower apparent pI and basic species are variants with higher apparent pI when antibodies are analyzed using IEF based methods.

The term “residence time” typically refers to the time which a particular product molecule is present in a bioreactor, i.e. the time spanning from its biotechnological generation until its separation from the bioreactor lumen.

The “product quality” is typically assessed by the presence or absence of clipping, degradation, deamidation and/or aggregation. For example, a product (molecule) comprising a percentile content of HMW species below 40%, preferably below 35, or even 30, 25 or 20% may be considered as of preferred product quality. Also, preferred product quality is associated with the essential absence of residual Host Cell Protein (HCP) and the essential absence of clipping, degradation and deamidation, or with a significant reduction of HCP concentration, clipping, degradation and/or deamidation in comparison to a product manufactured by a process different than the process of the present invention, such as a fed-batch process. Methods known in the art to assess product quality in the context of the present invention comprise Cation Exchange-High Performance Chromatography for Charge Variant Analysis (CEX-HPLC), Tryptic Peptide Mapping for Chemical Modifications, Host Cell Protein (HCP) ELISA Reduced Capillary Electrophoresis-Sodium Dodecyl Sulfate (RCE-SDS), and Size Exclusion-High Performance Liquid Chromatography (SE-HPLC).

The term “product” refers to “secreted protein” or “secreted recombinant protein” and means a protein (e.g., a recombinant protein) that originally contained at least one secretion signal sequence when it is translated within a mammalian cell, and through, at least in part, enzymatic cleavage of the secretion signal sequence in the mammalian cell, is secreted at least partially into the extracellular space (e.g., a liquid culture medium). Skilled practitioners will appreciate that a “secreted” protein need not dissociate entirely from the cell to be considered a secreted protein.

The term “polypeptide” is understood herein as an organic polymer which comprises at least one continuous, unbranched amino acid chain. In the context of the present invention, a polypeptide comprising more than one amino acid chain is likewise envisaged. An amino acid chain of a polypeptide typically comprises at least 50 amino acids, preferably at least 100, 200, 300, 400 or 500 amino acids. It is also envisaged in the context of the present invention that an amino acid chain of a polymer is linked to an entity which is not composed of amino acids.

The term “antigen-binding polypeptide” according to the present invention is preferably a polypeptide which immunospecifically binds to its target or antigen. It typically comprises the heavy chain variable region (VH) and/or the light chain variable region (VL) of an antibody, or comprises domains derived therefrom. A polypeptide according to the invention comprises the minimum structural requirements of an antibody which allow for immunospecific target binding. This minimum requirement may e.g. be defined by the presence of at least three light chain CDRs (i.e. CDR1, CDR2 and CDR3 of the VL region) and/or three heavy chain CDRs (i.e. CDR1, CDR2 and CDR3 of the VH region), preferably of all six CDRs. A T-cell engaging polypeptide may hence be characterized by the presence of three or six CDRs in either one or both binding domains, and the skilled person knows where (in which order) those CDRs are located within the binding domain

The term “bispecific antigen-binding polypeptide product” encompasses bispecific antibodies such as full length e.g. IgG-based antibodies as well as fragments therefor, which are typically referred to herein as bispecific antigen-binding polypeptides.

Alternatively, in the context of the present invention, an antigen-binding polypeptide like an “antibody construct” refers to a molecule in which the structure and/or function is/are based on the structure and/or function of an antibody, e.g., of a full-length or whole immunoglobulin molecule (typically comprising of two untruncated heavy and two light chains) and/or is/are drawn from the variable heavy chain (VH) and/or variable light chain (VL) domains of an antibody or fragment thereof. An antigen-binding polypeptide is hence capable of binding to its specific target or antigen. Furthermore, the domain which binds to its binding partner according to the present invention is understood herein as a binding domain of an antigen-binding polypeptide according to the invention. Typically, a binding domain according to the present invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope of the antibody within the structure of the specific target, respectively, the protein domain of the target protein composing the epitope region (epitope cluster) or by reference to an specific antibody competing with the epitope of the defined antibody. The antibodies on which the constructs according to the invention are based include for example monoclonal, recombinant, chimeric, deimmunized, humanized and human antibodies.

The binding domain of an antigen-binding polypeptide according to the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Examples for embodiments of antigen-binding polypeptides according to the invention are e.g. described in WO 00/006605, WO 2005/040220, WO 2008/119567, WO 2010/037838, WO 2013/026837, WO 2013/026833, US 2014/0308285, US 2014/0302037, WO 2014/144722, WO 2014/151910, and WO 2015/048272.

Also within the definition of “binding domain” or “domain which binds” are fragments of full-length antibodies, such as VH, VHH, VL, (s)dAb, Fv, Fd, Fab, Fab′, F(ab′)2 or “r IgG” (“half antibody”). Antigen-binding polypeptides according to the invention may also comprise modified fragments of antibodies, also called antibody variants, such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which might be VHH, VH or VL, that specifically bind an antigen or epitope independently of other V regions or domains.

As used herein, the terms “single-chain Fv,” “single-chain antibodies” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic.

Furthermore, the definition of the term “antigen-binding polypeptide” includes monovalent, bivalent and polyvalent/multivalent constructs and, thus, bispecific constructs, specifically binding to only two antigenic structure, as well as polyspecific/multispecific constructs, which specifically bind more than two antigenic structures, e.g. three, four or more, through distinct binding domains. Moreover, the definition of the term “antigen-binding polypeptide” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dübel, Antibody Engineering, Springer, 2nd ed. 2010 and Little, Recombinant Antibodies for Immunotherapy, Cambridge University Press 2009.

The term “bispecific” as used herein refers to an antigen-binding polypeptide which is “at least bispecific”, i.e., it comprises at least a first binding domain and a second binding domain, wherein the first binding domain binds to one antigen or target (e.g. the target cell surface antigen), and the second binding domain binds to another antigen or target (e.g. CD3). Accordingly, antigen-binding polypeptides according to the invention comprise specificities for at least two different antigens or targets. For example, the first domain does preferably not bind to an extracellular epitope of CD3ε of one or more of the species as described herein. The term “target cell surface antigen” refers to an antigenic structure expressed by a cell and which is present at the cell surface such that it is accessible for an antigen-binding polypeptide as described herein. It may be a protein, preferably the extracellular portion of a protein, or a carbohydrate structure, preferably a carbohydrate structure of a protein, such as a glycoprotein. It is preferably a tumor antigen. The term “bispecific antigen-binding polypeptide” of the invention also encompasses multispecific antigen-binding polypeptides such as trispecific antigen-binding polypeptides, the latter ones including three binding domains, or constructs having more than three (e.g. four, five . . . ) specificities.

A T-cell engaging antigen-binding polypeptide according to the present invention is preferably bispecific which is understood herein to typically comprise one domain binding to at least one target antigen and another domain binding to CD3. Hence, it does not occur naturally, and it is markedly different in its function from naturally occurring products. A polypeptide in accordance with the invention is hence an artificial “hybrid” polypeptide comprising at least two distinct binding domains with different specificities and is, thus, bispecific. Bispecific antigen-binding polypeptides can be produced by a variety of methods including fusion of hybridomas or linking of Fab′ fragments. See, e.g., Songsivilai & Lachmann, Clin. Exp. Immunol. 79:315-321 (1990).

The at least two binding domains and the variable domains (VH/VL) of the antigen-binding polypeptide of the present invention may or may not comprise peptide linkers (spacer peptides). The term “peptide linker” comprises in accordance with the present invention an amino acid sequence by which the amino acid sequences of one (variable and/or binding) domain and another (variable and/or binding) domain of the antigen-binding polypeptide of the invention are linked with each other. The peptide linkers can also be used to fuse the third domain to the other domains of the antigen-binding polypeptide of the invention. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antigen-binding polypeptide of the invention.

The antigen-binding polypeptides of the present invention are preferably “in vitro generated antigen-binding polypeptides”. This term refers to an antigen-binding polypeptide according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

The term “monoclonal antibody” (mAb) or monoclonal antibody from which a antigen-binding polypeptide as used herein is derived refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target cell surface antigen, (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antigen-binding polypeptides include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dübel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antigen-binding polypeptides, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antigen-binding polypeptides involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human target cell surface antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antigen-binding polypeptides of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primatized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. Sci U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antigen-binding polypeptide, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class Il binding peptides can be searched for motifs present in the VH and VL sequences, as described in WO 98/52976 and WO 00/34317. These motifs bind to any of the 18 major MHC class Il DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. MoI. Biol. 227:776-798; Cook, G. P. et al. (1995) Immunol. Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antigen-binding polypeptides, variants or fragments thereof (such as Fv, Fab, Fab′, F(ab′)2 or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

The term “human antibody”, “human antigen-binding polypeptide” and “human binding domain” includes antibodies, antigen-binding polypeptides and binding domains having antibody regions such as variable and constant regions or domains which correspond substantially to human germline immunoglobulin sequences known in the art, including, for example, those described by Kabat et al. (1991) (loc. cit.). The human antibodies, antigen-binding polypeptides or binding domains of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antigen-binding polypeptides or binding domains can have at least one, two, three, four, five, or more positions replaced with an amino acid residue that is not encoded by the human germline immunoglobulin sequence. The definition of human antibodies, antigen-binding polypeptides and binding domains as used herein, however, also contemplates “fully human antibodies”, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences

In some embodiments, the antigen-binding polypeptides of the invention are “isolated” or “substantially pure” antigen-binding polypeptides. “Isolated” or “substantially pure”, when used to describe the antigen-binding polypeptides disclosed herein, means an antigen-binding polypeptide that has been identified, separated and/or recovered from a component of its production environment. Preferably, the antigen-binding polypeptide is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antigen-binding polypeptides may e.g constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antigen-binding polypeptide in a wide variety of organisms and/or host cells that are known in the art. In preferred embodiments, the antigen-binding polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antigen-binding polypeptide will be prepared by at least one purification step.

The term “binding domain” characterizes in connection with the present invention a domain which (specifically) binds to/interacts with/recognizes a given target epitope or a given target side on the target molecules (antigens), e.g. CD33 and CD3, respectively. The structure and function of the first binding domain (recognizing e.g. CD33), and preferably also the structure and/or function of the second binding domain (recognizing e.g. CD3), is/are based on the structure and/or function of an antibody, e.g. of a full-length or whole immunoglobulin molecule and/or is/are drawn from the variable heavy chain (VH) and/or variable light chain (VL) domains of an antibody or fragment thereof. Preferably the first binding domain is characterized by the presence of three light chain CDRs (i.e. CDR1, CDR2 and CDR3 of the VL region) and/or three heavy chain CDRs (i.e. CDR1, CDR2 and CDR3 of the VH region). The second binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

Preferably the binding domain which binds to the target cell surface antigen and/or the binding domain which binds to CD3ε is/are human binding domains. Antibodies and antigen-binding polypeptides comprising at least one human binding domain avoid some of the problems associated with antibodies or antigen-binding polypeptides that possess non-human such as rodent (e.g. murine, rat, hamster or rabbit) variable and/or constant regions. The presence of such rodent derived proteins can lead to the rapid clearance of the antibodies or antigen-binding polypeptides or can lead to the generation of an immune response against the antibody or antigen-binding polypeptide by a patient. In order to avoid the use of rodent derived antibodies or antigen-binding polypeptides, human or fully human antibodies/antigen-binding polypeptides can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antigen-binding polypeptides are expected to minimize the immunogenic and allergic responses intrinsic to mouse or mouse-derivatized mAbs and thus to increase the efficacy and safety of the administered antibodies/antigen-binding polypeptides. The use of fully human antibodies or antigen-binding polypeptides can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse mice is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antigen-binding polypeptides comprising a human binding domain against the target cell surface antigen and a human binding domain against CD3ε in order to vitiate concerns and/or effects of HAMA or HACA response.

The terms “(specifically) binds to”, (specifically) recognizes”, “is (specifically) directed to”, and “(specifically) reacts with” mean in accordance with this invention that a binding domain interacts or specifically interacts with a given epitope or a given target side on the target molecules (antigens), here: target cell surface antigen and CD3ε, respectively.

The term “epitope” refers to a side on an antigen to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction side”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

A method for epitope mapping is described in the following: When a region (a contiguous amino acid stretch) in the human target cell surface antigen protein is exchanged/replaced with its corresponding region of a non-human and non-primate target cell surface antigen (e.g., mouse target cell surface antigen, but others like chicken, rat, hamster, rabbit etc. might also be conceivable), a decrease in the binding of the binding domain is expected to occur, unless the binding domain is cross-reactive for the non-human, non-primate target cell surface antigen used. Said decrease is preferably at least 10%, 20%, 30%, 40%, or 50%; more preferably at least 60%, 70%, or 80%, and most preferably 90%, 95% or even 100% in comparison to the binding to the respective region in the human target cell surface antigen protein, whereby binding to the respective region in the human target cell surface antigen protein is set to be 100%. It is envisaged that the aforementioned human target cell surface antigen/non-human target cell surface antigen chimeras are expressed in CHO cells. It is also envisaged that the human target cell surface antigen/non-human target cell surface antigen chimeras are fused with a transmembrane domain and/or cytoplasmic domain of a different membrane-bound protein such as EpCAM.

In an alternative or additional method for epitope mapping, several truncated versions of the human target cell surface antigen extracellular domain can be generated in order to determine a specific region that is recognized by a binding domain. In these truncated versions, the different extracellular target cell surface antigen domains/sub-domains or regions are stepwise deleted, starting from the N-terminus. It is envisaged that the truncated target cell surface antigen versions may be expressed in CHO cells. It is also envisaged that the truncated target cell surface antigen versions may be fused with a transmembrane domain and/or cytoplasmic domain of a different membrane-bound protein such as EpCAM. It is also envisaged that the truncated target cell surface antigen versions may encompass a signal peptide domain at their N-terminus, for example a signal peptide derived from mouse IgG heavy chain signal peptide. It is furthermore envisaged that the truncated target cell surface antigen versions may encompass a v5 domain at their N-terminus (following the signal peptide) which allows verifying their correct expression on the cell surface. A decrease or a loss of binding is expected to occur with those truncated target cell surface antigen versions which do not encompass any more the target cell surface antigen region that is recognized by the binding domain. The decrease of binding is preferably at least 10%, 20%, 30%, 40%, 50%; more preferably at least 60%, 70%, 80%, and most preferably 90%, 95% or even 100%, whereby binding to the entire human target cell surface antigen protein (or its extracellular region or domain) is set to be 100.

A further method to determine the contribution of a specific residue of a target cell surface antigen to the recognition by an antigen-binding polypeptide or binding domain is alanine scanning (see e.g. Morrison K L & Weiss G A. Cur Opin Chem Biol. 2001 June; 5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired. Alanine scanning is a mature technology which has been used for a long period of time.

The interaction between the binding domain and the epitope or the region comprising the epitope implies that a binding domain exhibits appreciable affinity for the epitope/the region comprising the epitope on a particular protein or antigen (here: target cell surface antigen and CD3, respectively) and, generally, does not exhibit significant reactivity with proteins or antigens other than the target cell surface antigen or CD3. “Appreciable affinity” includes binding with an affinity of about 10⁻⁶ M (KD) or stronger. Preferably, binding is considered specific when the binding affinity is about 10⁻¹² to 10⁻⁸ M, 10⁻¹² to 10⁻⁹ M, 10⁻¹² to 10⁻¹⁰ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than the target cell surface antigen or CD3. Preferably, a binding domain of the invention does not essentially or substantially bind to proteins or antigens other than the target cell surface antigen or CD3 (i.e., the first binding domain is preferably not capable of binding to proteins other than the target cell surface antigen and the second binding domain is not capable of binding to proteins other than CD3). It is an envisaged characteristic of the antigen-binding polypeptides according to the present invention to have superior affinity characteristics in comparison to other HLE formats. Such a superior affinity, in consequence, suggests a prolonged half-life in vivo. The longer half-life of the antigen-binding polypeptides according to the present invention may reduce the duration and frequency of administration which typically contributes to improved patient compliance. This is of particular importance as the antigen-binding polypeptides of the present invention are particularly beneficial for highly weakened or even multimorbide cancer patients.

The term “does not essentially/substantially bind” or “is not capable of binding” means that a binding domain of the present invention does not bind a protein or antigen other than the target cell surface antigen or CD3, i.e., does not show reactivity of more than 30%, preferably not more than 20%, more preferably not more than 10%, particularly preferably not more than 9%, 8%, 7%, 6% or 5% with proteins or antigens other than the target cell surface antigen or CD3, whereby binding to the target cell surface antigen or CD3, respectively, is set to be 100%.

Specific binding is believed to be effected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding side.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a f-sheet configuration, connected by three hypervariable regions, which form loops connecting, and in some cases forming part of, the f-sheet structure. The hypervariable regions in each chain are held together in close proximity by the FRM and, with the hypervariable regions from the other chain, contribute to the formation of the antigen-binding side (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. MoI. Biol, 1987, 196: 901-917; and MacCallum et al., J. MoI. Biol, 1996, 262: 732). Still another standard for characterizing the antigen binding side is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. MoI. Biol., 1987, 196: 901; Chothia et al., Nature, 1989, 342: 877; Martin and Thornton, J. MoI. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antigen-binding polypeptides, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding side. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 10¹⁰ different antibody molecules (Immunoglobulin Genes, 2^{nd} ed., eds. Jonio et al., Academic Press, San Diego, Calif., 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The term “Fc portion” or “Fc monomer” means in connection with this invention a polypeptide comprising at least one domain having the function of a CH2 domain and at least one domain having the function of a CH3 domain of an immunoglobulin molecule. As apparent from the term “Fc monomer”, the polypeptide comprising those CH domains is a “polypeptide monomer”. An Fc monomer can be a polypeptide comprising at least a fragment of the constant region of an immunoglobulin excluding the first constant region immunoglobulin domain of the heavy chain (CH1), but maintaining at least a functional part of one CH2 domain and a functional part of one CH3 domain, wherein the CH2 domain is amino terminal to the CH3 domain. In a preferred aspect of this definition, an Fc monomer can be a polypeptide constant region comprising a portion of the Ig-Fc hinge region, a CH2 region and a CH3 region, wherein the hinge region is amino terminal to the CH2 domain. It is envisaged that the hinge region of the present invention promotes dimerization. Such Fc polypeptide molecules can be obtained by papain digestion of an immunoglobulin region (of course resulting in a dimer of two Fc polypeptide), for example and not limitation. In another aspect of this definition, an Fc monomer can be a polypeptide region comprising a portion of a CH2 region and a CH3 region. Such Fc polypeptide molecules can be obtained by pepsin digestion of an immunoglobulin molecule, for example and not limitation. In one embodiment, the polypeptide sequence of an Fc monomer is substantially similar to an Fc polypeptide sequence of: an IgG₁ Fc region, an IgG₂ Fc region, an IgG₃ Fc region, an IgG₄ Fc region, an IgM Fc region, an IgA Fc region, an IgD Fc region and an IgE Fc region. (See, e.g., Padlan, Molecular Immunology, 31(3), 169-217 (1993)). Because there is some variation between immunoglobulins, and solely for clarity, Fc monomer refers to the last two heavy chain constant region immunoglobulin domains of IgA, IgD, and IgG, and the last three heavy chain constant region immunoglobulin domains of IgE and IgM. As mentioned, the Fc monomer can also include the flexible hinge N-terminal to these domains. For IgA and IgM, the Fc monomer may include the J chain. For IgG, the Fc portion comprises immunoglobulin domains CH2 and CH3 and the hinge between the first two domains and CH2. Although the boundaries of the Fc portion may vary an example for a human IgG heavy chain Fc portion comprising a functional hinge, CH2 and CH3 domain can be defined e.g. to comprise residues D231 (of the hinge domain—corresponding to D234 in Table 1 below)) to P476, respectively L476 (for IgG₄) of the carboxyl-terminus of the CH3 domain, wherein the numbering is according to Kabat. The two Fc portions or Fc monomers, which are fused to each other via a peptide linker define the third domain of the antigen-binding polypeptide of the invention, which may also be defined as scFc domain.

In one embodiment of the invention it is envisaged that a scFc domain as disclosed herein, respectively the Fc monomers fused to each other are comprised only in the third domain of the antigen-binding polypeptide.

In line with the present invention an IgG hinge region can be identified by analogy using the Kabat numbering as set forth in Table 1. In line with the above, it is envisaged that a hinge domain/region of the present invention comprises the amino acid residues corresponding to the IgG₁ sequence stretch of D234 to P243 according to the Kabat numbering. It is likewise envisaged that a hinge domain/region of the present invention comprises or consists of the IgG1 hinge sequence DKTHTCPPCP (SEQ ID NO: 182) (corresponding to the stretch D234 to P243 as shown in Table 1 below—variations of said sequence are also envisaged provided that the hinge region still promotes dimerization). In a preferred embodiment of the invention the glycosylation site at Kabat position 314 of the CH2 domains in the third domain of the antigen-binding polypeptide is removed by a N314X substitution, wherein X is any amino acid excluding Q. Said substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).
It is also envisaged that the third domain of the antigen-binding polypeptide of the invention comprises or consists in an amino to carboxyl order: DKTHTCPPCP (SEQ ID NO: 182) (i.e. hinge)-CH2-CH3-linker-DKTHTCPPCP (SEQ ID NO: 182) (i.e. hinge)-CH2-CH3. The peptide linker of the aforementioned antigen-binding polypeptide is in a preferred embodiment characterized by the amino acid sequence Gly-Gly-Gly-Gly-Ser, i.e. Gly₄Ser (SEQ ID NO: 187), or polymers thereof, i.e. (Gly₄Ser)x, where x is an integer of 5 or greater (e.g. 5, 6, 7, 8 etc. or greater), 6 being preferred ((Gly4Ser)6). Said construct may further comprise the aforementioned substitutions N314X, preferably N314G, and/or the further substitutions V321C and R309C. In a preferred embodiment of the antigen-binding polypeptides of the invention as defined herein before, it is envisaged that the second domain binds to an extracellular epitope of the human and/or the Macaca CD3ε chain.

TABLE 1
Kabat numbering of the amino acid residues of the hinge region

| IMGT numbering | IgG₁ amino acid | Kabat |
|---|---|---|
| for the hinge | translation | numbering |
| 1 | I | 226 |
| 2 | P | 227 |
| 3 | K | 228 |
| 4 | S | 232 |
| 5 | C | 233 |
| 6 | D | 234 |
| 7 | K | 235 |
| 8 | T | 236 |
| 9 | H | 237 |
| 10 | T | 238 |
| 11 | C | 239 |
| 12 | P | 240 |
| 13 | P | 241 |
| 14 | C | 242 |
| 15 | P | 243 |

In further embodiments of the present invention, the hinge domain/region comprises or consists of the IgG2 subtype hinge sequence ERKCCVECPPCP (SEQ ID NO: 183), the IgG3 subtype hinge sequence ELKTPLDTTHTCPRCP (SEQ ID NO: 184) or ELKTPLGDTTHTCPRCP (SEQ ID NO: 185), and/or the IgG4 subtype hinge sequence ESKYGPPCPSCP (SEQ ID NO: 186). The IgG1 subtype hinge sequence may be the following one EPKSCDKTHTCPPCP (as shown in Table 1 and SEQ ID NO: 183). These core hinge regions are thus also envisaged in the context of the present invention.

The location and sequence of the IgG CH2 and IgG CD3 domain can be identified by analogy using the Kabat numbering as set forth in Table 2:

TABLE 2
Kabat numbering of the amino acid residues of the IgG CH2 and CH3 region

| IgG | CH2 aa | | CH2 Kabat | | CH3 aa | | CH3 Kabat | |
|---|---|---|---|---|---|---|---|---|
| subtype | translation | | numbering | | translation | | numbering | |
| IgG₁ | APE . . . | . . . KAK | 244 . . . | . . . 360 | GQP . . . | . . . PGK | 361 . . . | . . . 478 |
| IgG₂ | APP . . . | . . . KTK | 244 . . . | . . . 360 | GQP . . . | . . . PGK | 361 . . . | . . . 478 |
| IgG₃ | APE . . . | . . . KTK | 244 . . . | . . . 360 | GQP . . . | . . . PGK | 361 . . . | . . . 478 |
| IgG₄ | APE . . . | . . . KAK | 244 . . . | . . . 360 | GQP . . . | . . . LGK | 361 . . . | . . . 478 |

In one embodiment of the invention the emphasized bold amino acid residues in the CH3 domain of the first or both Fc monomers are deleted.

The peptide linker, by whom the polypeptide monomers (“Fc portion” or “Fc monomer”) of the third domain are fused to each other, preferably comprises at least 25 amino acid residues (25, 26, 27, 28, 29, 30 etc.). More preferably, this peptide linker comprises at least 30 amino acid residues (30, 31, 32, 33, 34, 35 etc.). It is also preferred that the linker comprises up to 40 amino acid residues, more preferably up to 35 amino acid residues, most preferably exactly 30 amino acid residues. A preferred embodiment of such peptide linker is characterized by the amino acid sequence Gly-Gly-Gly-Gly-Ser, i.e. Gly₄Ser (SEQ ID NO: 187), or polymers thereof, i.e. (Gly₄Ser)x, where x is an integer of 5 or greater (e.g. 6, 7 or 8). Preferably the integer is 6 or 7, more preferably the integer is 6.

In the event that a linker is used to fuse the first domain to the second domain, or the first or second domain to the third domain, this linker is preferably of a length and sequence sufficient to ensure that each of the first and second domains can, independently from one another, retain their differential binding specificities. For peptide linkers which connect the at least two binding domains (or two variable domains) in the antigen-binding polypeptide of the invention, those peptide linkers are preferred which comprise only a few number of amino acid residues, e.g. 12 amino acid residues or less. Thus, peptide linkers of 12, 11, 10, 9, 8, 7, 6 or 5 amino acid residues are preferred. An envisaged peptide linker with less than 5 amino acids comprises 4, 3, 2 or one amino acid(s), wherein Gly-rich linkers are preferred. A preferred embodiment of the peptide linker for a fusion the first and the second domain is depicted in SEQ ID NO:1. A preferred linker embodiment of the peptide linker for a fusion the second and the third domain is a (Gly)₄-linker, respectively G₄-linker.

A particularly preferred “single” amino acid in the context of one of the above described “peptide linker” is Gly. Accordingly, said peptide linker may consist of the single amino acid Gly. In a preferred embodiment of the invention a peptide linker is characterized by the amino acid sequence Gly-Gly-Gly-Gly-Ser, i.e. Gly₄Ser (SEQ ID NO: 187), or polymers thereof, i.e. (Gly₄Ser)x, where x is an integer of 1 or greater (e.g. 2 or 3). Preferred linkers are depicted in SEQ ID Nos: 1 to 12. The characteristics of said peptide linker, which comprise the absence of the promotion of secondary structures, are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which furthermore do not promote any secondary structures are preferred. The linkage of said domains to each other can be provided, e.g., by genetic engineering, as described in the examples. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, N.Y., 2001).

In a preferred embodiment of the antigen-binding polypeptide or the present invention the first and second domain form an antigen-binding polypeptide in a format selected from the group consisting of (scFv)₂, scFv-single domain mAb, diabody and oligomers of any of the those formats

According to a particularly preferred embodiment, and as documented in the appended examples, the first and the second domain of the antigen-binding polypeptide of the invention is a “bispecific single chain antigen-binding polypeptide”, more preferably a bispecific “single chain Fv” (scFv). Although the two domains of the Fv fragment, VL and VH, are coded for by separate genes, they can be joined, using recombinant methods, by a synthetic linker—as described hereinbefore—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are whole or full-length antibodies. A single-chain variable fragment (scFv) is hence a fusion protein of the variable region of the heavy chain (VH) and of the light chain (VL) of immunoglobulins, usually connected with a short linker peptide of about ten to about 25 amino acids, preferably about 15 to 20 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and introduction of the linker.

Bispecific single chain antigen-binding polypeptides are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56. Techniques described for the production of single chain antibodies (see, inter alia, U.S. Pat. No. 4,946,778, Kontermann and Dübel (2010), loc. Cit. and Little (2009), loc. Cit.) can be adapted to produce single chain antigen-binding polypeptides specifically recognizing (an) elected target(s).

Bivalent (also called divalent) or bispecific single-chain variable fragments (bi-scFvs or di-scFvs having the format (scFv)₂ can be engineered by linking two scFv molecules (e.g. with linkers as described hereinbefore). If these two scFv molecules have the same binding specificity, the resulting (scFv)₂ molecule will preferably be called bivalent (i.e. it has two valences for the same target epitope). If the two scFv molecules have different binding specificities, the resulting (scFv)₂ molecule will preferably be called bispecific. The linking can be done by producing a single peptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. This type is known as diabodies (see e.g. Hollinger, Philipp et al., (July 1993) Proceedings of the National Academy of Sciences of the United States of America 90 (14): 6444-8).

In line with this invention either the first, the second or the first and the second domain may comprise a single domain antibody, respectively the variable domain or at least the CDRs of a single domain antibody. Single domain antibodies comprise merely one (monomeric) antibody variable domain which is able to bind selectively to a specific antigen, independently of other V regions or domains. The first single domain antibodies were engineered from heavy chain antibodies found in camelids, and these are called V_{H}H fragments. Cartilaginous fishes also have heavy chain antibodies (IgNAR) from which single domain antibodies called V_{NAR} fragments can be obtained. An alternative approach is to split the dimeric variable domains from common immunoglobulins e.g. from humans or rodents into monomers, hence obtaining VH or VL as a single domain Ab. Although most research into single domain antibodies is currently based on heavy chain variable domains, nanobodies derived from light chains have also been shown to bind specifically to target epitopes. Examples of single domain antibodies are called sdAb, nanobodies or single variable domain antibodies.

A (single domain mAb)₂ is hence a monoclonal antigen-binding polypeptide composed of (at least) two single domain monoclonal antibodies, which are individually selected from the group comprising V_{H}, V_{L}, V_{H}H and V_{NAR}. The linker is preferably in the form of a peptide linker. Similarly, an “scFv-single domain mAb” is a monoclonal antigen-binding polypeptide composed of at least one single domain antibody as described above and one scFv molecule as described above. Again, the linker is preferably in the form of a peptide linker.

Whether or not an antigen-binding polypeptide competes for binding with another given antigen-binding polypeptide can be measured in a competition assay such as a competitive ELISA or a cell-based competition assay. Avidin-coupled microparticles (beads) can also be used. Similar to an avidin-coated ELISA plate, when reacted with a biotinylated protein, each of these beads can be used as a substrate on which an assay can be performed. Antigen is coated onto a bead and then precoated with the first antibody. The second antibody is added and any additional binding is determined. Possible means for the read-out includes flow cytometry.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

The CD3 receptor complex is a protein complex and is composed of four chains. In mammals, the complex contains a CD3γ (gamma) chain, a CD3δ (delta) chain, and two CD3ε (epsilon) chains. These chains associate with the T cell receptor (TCR) and the so-called ζ (zeta) chain to form the T cell receptor CD3 complex and to generate an activation signal in T lymphocytes. The CD3γ (gamma), CD3δ (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3E gene which resides on chromosome 11. The most preferred epitope of CD3 epsilon is comprised within amino acid residues 1-27 of the human CD3 epsilon extracellular domain. It is envisaged that antigen-binding polypeptides according to the present invention typically and advantageously show less unspecific T cell activation, which is not desired in specific immunotherapy. This translates to a reduced risk of side effects.

The redirected lysis of target cells via the recruitment of T cells by a multispecific, at least bispecific, antigen-binding polypeptide involves cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO 2007/042261.

Cytotoxicity mediated by antigen-binding polypeptides of the invention can be measured in various ways. Effector cells can be e.g. stimulated enriched (human) CD8 positive T cells or unstimulated (human) peripheral blood mononuclear cells (PBMC). If the target cells are of macaque origin or express or are transfected with macaque target cell surface antigen which is bound by the first domain, the effector cells should also be of macaque origin such as a macaque T cell line, e.g. 4119LnPx. The target cells should express (at least the extracellular domain of) the target cell surface antigen, e.g. human or macaque target cell surface antigen. Target cells can be a cell line (such as CHO) which is stably or transiently transfected with target cell surface antigen, e.g. human or macaque target cell surface antigen. Alternatively, the target cells can be a target cell surface antigen positive natural expresser cell line. Usually EC₅₀ values are expected to be lower with target cell lines expressing higher levels of target cell surface antigen on the cell surface. The effector to target cell (E:T) ratio is usually about 10:1, but can also vary. Cytotoxic activity of target cell surface antigen×CD3 bispecific antigen-binding polypeptides can be measured in a ⁵¹Cr-release assay (incubation time of about 18 hours) or in a in a FACS-based cytotoxicity assay (incubation time of about 48 hours). Modifications of the assay incubation time (cytotoxic reaction) are also possible. Other methods of measuring cytotoxicity are well-known to the skilled person and comprise MTT or MTS assays, ATP-based assays including bioluminescent assays, the sulforhodamine B (SRB) assay, WST assay, clonogenic assay and the ECIS technology.

The cytotoxic activity mediated by target cell surface antigen×CD3 bispecific antigen-binding polypeptides of the present invention is preferably measured in a cell-based cytotoxicity assay. It may also be measured in a ⁵¹Cr-release assay. It is represented by the EC₅₀ value, which corresponds to the half maximal effective concentration (concentration of the antigen-binding polypeptide which induces a cytotoxic response halfway between the baseline and maximum). Preferably, the EC₅₀ value of the target cell surface antigen×CD3 bispecific antigen-binding polypeptides is ≤5000 pM or ≤4000 pM, more preferably ≤3000 pM or ≤2000 pM, even more preferably ≤1000 pM or ≤500 pM, even more preferably ≤400 pM or ≤300 pM, even more preferably ≤200 pM, even more preferably ≤100 pM, even more preferably ≤50 pM, even more preferably ≤20 pM or ≤10 pM, and most preferably ≤5 pM.

The above given EC₅₀ values can be measured in different assays. The skilled person is aware that an EC₅₀ value can be expected to be lower when stimulated/enriched CD8⁺ T cells are used as effector cells, compared with unstimulated PBMC. It can furthermore be expected that the EC₅₀ values are lower when the target cells express a high number of the target cell surface antigen compared with a low target expression rat. For example, when stimulated/enriched human CD8⁺ T cells are used as effector cells (and either target cell surface antigen transfected cells such as CHO cells or target cell surface antigen positive human cell lines are used as target cells), the EC₅₀ value of the target cell surface antigen×CD3 bispecific antigen-binding polypeptide is preferably ≤1000 pM, more preferably ≤500 pM, even more preferably ≤250 pM, even more preferably ≤100 pM, even more preferably ≤50 pM, even more preferably ≤10 pM, and most preferably ≤5 pM. When human PBMCs are used as effector cells, the EC₅₀ value of the target cell surface antigen×CD3 bispecific antigen-binding polypeptide is preferably ≤5000 pM or ≤4000 pM (in particular when the target cells are target cell surface antigen positive human cell lines), more preferably ≤2000 pM (in particular when the target cells are target cell surface antigen transfected cells such as CHO cells), more preferably ≤1000 pM or ≤500 pM, even more preferably ≤200 pM, even more preferably ≤150 pM, even more preferably ≤100 pM, and most preferably ≤50 pM, or lower. When a macaque T cell line such as LnP×4119 is used as effector cells, and a macaque target cell surface antigen transfected cell line such as CHO cells is used as target cell line, the EC₅₀ value of the target cell surface antigen×CD3 bispecific antigen-binding polypeptide is preferably ≤2000 pM or ≤1500 pM, more preferably ≤1000 pM or ≤500 pM, even more preferably ≤300 pM or ≤250 pM, even more preferably ≤100 pM, and most preferably ≤50 pM.

Preferably, the target cell surface antigen×CD3 bispecific antigen-binding polypeptides of the present invention do not induce/mediate lysis or do not essentially induce/mediate lysis of target cell surface antigen negative cells such as CHO cells. The term “do not induce lysis”, “do not essentially induce lysis”, “do not mediate lysis” or “do not essentially mediate lysis” means that an antigen-binding polypeptide of the present invention does not induce or mediate lysis of more than 30%, preferably not more than 20%, more preferably not more than 10%, particularly preferably not more than 9%, 8%, 7%, 6% or 5% of target cell surface antigen negative cells, whereby lysis of a target cell surface antigen positive human cell line is set to be 100%. This usually applies for concentrations of the antigen-binding polypeptide of up to 500 nM. The skilled person knows how to measure cell lysis without further ado. Moreover, the present specification teaches specific instructions how to measure cell lysis.

The difference in cytotoxic activity between the monomeric and the dimeric isoform of individual target cell surface antigen×CD3 bispecific antigen-binding polypeptides is referred to as “potency gap”. This potency gap can e.g. be calculated as ratio between EC₅₀ values of the molecule's monomeric and dimeric form. Potency gaps of the target cell surface antigen×CD3 bispecific antigen-binding polypeptides of the present invention are preferably ≤5, more preferably ≤4, even more preferably ≤3, even more preferably ≤2 and most preferably ≤1.

The first and/or the second (or any further) binding domain(s) of the antigen-binding polypeptide of the invention is/are preferably cross-species specific for members of the mammalian order of primates. Cross-species specific CD3 binding domains are, for example, described in WO 2008/119567. According to one embodiment, the first and/or second binding domain, in addition to binding to human target cell surface antigen and human CD3, respectively, will also bind to target cell surface antigen/CD3 of primates including (but not limited to) new world primates (such as Callithrix jacchus, Saguinus oedipus or Saimiri sciureus), old world primates (such baboons and macaques), gibbons, and non-human homininae.

In one embodiment of the antigen-binding polypeptide of the invention the first domain binds to human target cell surface antigen and further binds to macaque target cell surface antigen, such as target cell surface antigen of Macaca fascicularis, and more preferably, to macaque target cell surface antigen expressed on the surface macaque cells. The affinity of the first binding domain for macaque target cell surface antigen is preferably ≤15 nM, more preferably ≤10 nM, even more preferably ≤5 nM, even more preferably ≤1 nM, even more preferably ≤0.5 nM, even more preferably ≤0.1 nM, and most preferably ≤0.05 nM or even ≤0.01 nM.

Preferably the affinity gap of the antigen-binding polypeptides according to the invention for binding macaque target cell surface antigen versus human target cell surface antigen [ma target cell surface antigen:hu target cell surface antigen] (as determined e.g. by BiaCore or by Scatchard analysis) is <100, preferably <20, more preferably <15, further preferably <10, even more preferably <8, more preferably <6 and most preferably <2. Preferred ranges for the affinity gap of the antigen-binding polypeptides according to the invention for binding macaque target cell surface antigen versus human target cell surface antigen are between 0.1 and 20, more preferably between 0.2 and 10, even more preferably between 0.3 and 6, even more preferably between 0.5 and 3 or between 0.5 and 2.5, and most preferably between 0.5 and 2 or between 0.6 and 2.

The second (binding) domain of the antigen-binding polypeptide of the invention binds to human CD3 epsilon and/or to Macaca CD3 epsilon. In a preferred embodiment the second domain further bind to Callithrix jacchus, Saguinus oedipus or Saimiri sciureus CD3 epsilon. Callithrix jacchus and Saguinus 40yophil are both new world primate belonging to the family of Callitrichidae, while Saimiri sciureus is a new world primate belonging to the family of Cebidae.

It is preferred for the antigen-binding polypeptide of the present invention that the second domain which binds to an extracellular epitope of the human and/or the Macaca CD3 on the comprises a VL region comprising CDR-L1, CDR-L2 and CDR-L3 selected from:

(a) CDR-L1 as depicted in SEQ ID NO: 27 of WO 2008/119567, CDR-L2 as depicted in SEQ ID NO: 28 of WO 2008/119567 and CDR-L3 as depicted in SEQ ID NO: 29 of WO 2008/119567;
(b) CDR-L1 as depicted in SEQ ID NO: 117 of WO 2008/119567, CDR-L2 as depicted in SEQ ID NO: 118 of WO 2008/119567 and CDR-L3 as depicted in SEQ ID NO: 119 of WO 2008/119567; and
I CDR-L1 as depicted in SEQ ID NO: 153 of WO 2008/119567, CDR-L2 as depicted in SEQ ID NO: 154 of WO 2008/119567 and CDR-L3 as depicted in SEQ ID NO: 155 of WO 2008/119567.

In an also preferred embodiment of the antigen-binding polypeptide of the present invention, the second domain which binds to an extracellular epitope of the human and/or the Macaca CD3 epsilon chain comprises a VH region comprising CDR-H 1, CDR-H2 and CDR-H3 selected from:

(a) CDR-H1 as depicted in SEQ ID NO: 12 of WO 2008/119567, CDR-H2 as depicted in SEQ ID NO: 13 of WO 2008/119567 and CDR-H3 as depicted in SEQ ID NO: 14 of WO 2008/119567;
(b) CDR-H1 as depicted in SEQ ID NO: 30 of WO 2008/119567, CDR-H2 as depicted in SEQ ID NO: 31 of WO 2008/119567 and CDR-H3 as depicted in SEQ ID NO: 32 of WO 2008/119567;
I CDR-H1 as depicted in SEQ ID NO: 48 of WO 2008/119567, CDR-H2 as depicted in SEQ ID NO: 49 of WO 2008/119567 and CDR-H3 as depicted in SEQ ID NO: 50 of WO 2008/119567;
(d) CDR-H1 as depicted in SEQ ID NO: 66 of WO 2008/119567, CDR-H2 as depicted in SEQ ID NO: 67 of WO 2008/119567 and CDR-H3 as depicted in SEQ ID NO: 68 of WO 2008/119567;
I CDR-H1 as depicted in SEQ ID NO: 84 of WO 2008/119567, CDR-H2 as depicted in SEQ ID NO: 85 of WO 2008/119567 and CDR-H3 as depicted in SEQ ID NO: 86 of WO 2008/119567;
(f) CDR-H1 as depicted in SEQ ID NO: 102 of WO 2008/119567, CDR-H2 as depicted in SEQ ID NO: 103 of WO 2008/119567 and CDR-H3 as depicted in SEQ ID NO: 104 of WO 2008/119567;
(g) CDR-H1 as depicted in SEQ ID NO: 120 of WO 2008/119567, CDR-H2 as depicted in SEQ ID NO: 121 of WO 2008/119567 and CDR-H3 as depicted in SEQ ID NO: 122 of WO 2008/119567;
(h) CDR-H1 as depicted in SEQ ID NO: 138 of WO 2008/119567, CDR-H2 as depicted in SEQ ID NO: 139 of WO 2008/119567 and CDR-H3 as depicted in SEQ ID NO: 140 of WO 2008/119567;
(i) CDR-H1 as depicted in SEQ ID NO: 156 of WO 2008/119567, CDR-H2 as depicted in SEQ ID NO: 157 of WO 2008/119567 and CDR-H3 as depicted in SEQ ID NO: 158 of WO 2008/119567; and
(j) CDR-H1 as depicted in SEQ ID NO: 174 of WO 2008/119567, CDR-H2 as depicted in SEQ ID NO: 175 of WO 2008/119567 and CDR-H3 as depicted in SEQ ID NO: 176 of WO 2008/119567.

In a preferred embodiment of the antigen-binding polypeptide of the invention the above described three groups of VL CDRs are combined with the above described ten groups of VH CDRs within the second binding domain to form (30) groups, each comprising CDR-L 1-3 and CDR-H 1-3.

It is preferred for the antigen-binding polypeptide of the present invention that the second domain which binds to CD3 comprises a VL region selected from the group consisting of a VL region as depicted in SEQ ID NO: 17, 21, 35, 39, 53, 57, 71, 75, 89, 93, 107, 111, 125, 129, 143, 147, 161, 165, 179 or 183 of WO 2008/119567 or as depicted in SEQ ID NO: 200.

It is also preferred that the second domain which binds to CD3 comprises a VH region selected from the group consisting of a VH region as depicted in SEQ ID NO: 15, 19, 33, 37, 51, 55, 69, 73, 87, 91, 105, 109, 123, 127, 141, 145, 159, 163, 177 or 181 of WO 2008/119567 or as depicted in SEQ ID NO: 201.

More preferably, the antigen-binding polypeptide of the present invention is characterized by a second domain which binds to CD3 comprising a VL region and a VH region selected from the group consisting of:

(a) a VL region as depicted in SEQ ID NO: 17 or 21 of WO 2008/119567 and a VH region as depicted in SEQ ID NO: 15 or 19 of WO 2008/119567;
(b) a VL region as depicted in SEQ ID NO: 35 or 39 of WO 2008/119567 and a VH region as depicted in SEQ ID NO: 33 or 37 of WO 2008/119567;
I a VL region as depicted in SEQ ID NO: 53 or 57 of WO 2008/119567 and a VH region as depicted in SEQ ID NO: 51 or 55 of WO 2008/119567;
(d) a VL region as depicted in SEQ ID NO: 71 or 75 of WO 2008/119567 and a VH region as depicted in SEQ ID NO: 69 or 73 of WO 2008/119567;
I a VL region as depicted in SEQ ID NO: 89 or 93 of WO 2008/119567 and a VH region as depicted in SEQ ID NO: 87 or 91 of WO 2008/119567;
(f) a VL region as depicted in SEQ ID NO: 107 or 111 of WO 2008/119567 and a VH region as depicted in SEQ ID NO: 105 or 109 of WO 2008/119567;
(g) a VL region as depicted in SEQ ID NO: 125 or 129 of WO 2008/119567 and a VH region as depicted in SEQ ID NO: 123 or 127 of WO 2008/119567;
(h) a VL region as depicted in SEQ ID NO: 143 or 147 of WO 2008/119567 and a VH region as depicted in SEQ ID NO: 141 or 145 of WO 2008/119567;
(i) a VL region as depicted in SEQ ID NO: 161 or 165 of WO 2008/119567 and a VH region as depicted in SEQ ID NO: 159 or 163 of WO 2008/119567; and
(j) a VL region as depicted in SEQ ID NO: 179 or 183 of WO 2008/119567 and a VH region as depicted in SEQ ID NO: 177 or 181 of WO 2008/119567.

Also preferred in connection with the antigen-binding polypeptide of the present invention is a second domain which binds to CD3 comprising a VL region as depicted in SEQ ID NO: 200 and a VH region as depicted in SEQ ID NO: 201.

According to a preferred embodiment of the antibody construct of the present invention, the first and/or the second domain have the following format: The pairs of VH regions and VL regions are in the format of a single chain antibody (scFv). The VH and VL regions are arranged in the order VH-VL or VL-VH. It is preferred that the VH-region is positioned N-terminally of a linker sequence, and the VL-region is positioned C-terminally of the linker sequence.

A preferred embodiment of the above described antibody construct of the present invention is characterized by the second domain which binds to CD3 comprising an amino acid sequence selected from the group consisting of SEQ ID Nos: 23, 25, 41, 43, 59, 61, 77, 79, 95, 97, 113, 115, 131, 133, 149, 151, 167, 169, 185 or 187 of WO 2008/119567 or depicted in SEQ ID NO: 202.

Covalent modifications of the antibody constructs are also included within the scope of this invention, and are generally, but not always, done post-translationally. For example, several types of covalent modifications of the antibody construct are introduced into the molecule by reacting specific amino acid residues of the antibody construct with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking the antibody constructs of the present invention to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates as described in U.S. Pat. Nos. 3,969,287; 3,691,016; 4,195,128; 4,247,642; 4,229,537; and 4,330,440 are employed for protein immobilization.

Other modifications of the antigen-binding polypeptide are also contemplated herein. For example, another type of covalent modification of the antigen-binding polypeptide comprises linking the antigen-binding polypeptide to various non-proteinaceous polymers, including, but not limited to, various polyols such as polyethylene glycol, polypropylene glycol, polyoxyalkylenes, or copolymers of polyethylene glycol and polypropylene glycol, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antigen-binding polypeptide, e.g. in order to facilitate the addition of polymers such as PEG.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8^{th} Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Nat. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804,387; 5,874,304; 5,876,995; 5,925,558).

The antibody construct of the invention may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed antibody constructs characterized by the identified CDRs may comprise a His-tag domain, which is generally known as a repeat of consecutive His residues in the amino acid sequence of a molecule, preferably of five, and more preferably of six His residues (hexa-histidine). The His-tag may be located e.g. at the N- or C-terminus of the antibody construct, preferably it is located at the C-terminus. Most preferably, a hexa-histidine tag (HHHHHH) (SEQ ID NO:199) is linked via peptide bond to the C-terminus of the antibody construct according to the invention. Additionally, a conjugate system of PLGA-PEG-PLGA may be combined with a poly-histidine tag for sustained release application and improved pharmacokinetic profile.

Amino acid sequence modifications of the antibody constructs described herein are also contemplated. For example, it may be desirable to improve the binding affinity and/or other biological properties of the antibody construct. Amino acid sequence variants of the antibody constructs are prepared by introducing appropriate nucleotide changes into the antibody constructs nucleic acid, or by peptide synthesis. All of the below described amino acid sequence modifications should result in an antibody construct which still retains the desired biological activity (binding to the target cell surface antigen and to CD3) of the unmodified parental molecule.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gin or Q); glutamic acid (Giu or E); glycine (Giy or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Giu); a positively charged sidechain (e.g., Arg, His, Lys); or an uncharged polar side chain (e.g., Asn, Cys, Gin, Giy, His, Met, Phe, Ser, Thr, Trp, and Tyr).

Amino acid modifications include, for example, deletions from, and/or insertions into, and/or substitutions of, residues within the amino acid sequences of the antibody constructs. Any combination of deletion, insertion, and substitution is made to arrive at the final construct, provided that the final construct possesses the desired characteristics. The amino acid changes also may alter post-translational processes of the antibody constructs, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the antibody construct include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. Corresponding modifications may also performed within the third domain of the antibody construct of the invention. An insertional variant of the antibody construct of the invention includes the fusion to the N-terminus or to the C-terminus of the antibody construct of an enzyme or the fusion to a polypeptide.

The sites of greatest interest for substitutional mutagenesis include (but are not limited to) the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the antibody constructs that are preferred locations for mutagenesis is called “alanine scanning mutagenesis” as described by Cunningham and Wells in Science, 244: 1081-1085 (1989). Here, a residue or group of target residues within the antibody construct is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as the target cell surface antigen or CD3 binding.

Generally, if amino acids are substituted in one or more or all of the CDRs of the heavy and/or light chain, it is preferred that the then-obtained “substituted” sequence is at least 60% or 65%, more preferably 70% or 75%, even more preferably 80% or 85%, and particularly preferably 90% or 95% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the antibody construct may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions” listed in Table 3, below) is envisaged as long as the antibody construct retains its capability to bind to the target cell surface antigen via the first domain and to CD3, respectively CD3 epsilon, via the second domain and/or its CDRs have an identity to the then substituted sequence (at least 60% or 65%, more preferably 70% or 75%, even more preferably 80% or 85%, and particularly preferably 90% or 95% identical to the “original” CDR sequence).

Conservative substitutions are shown in Table 3 under the heading of “preferred substitutions”. If such substitutions result in a change in biological activity, then more substantial changes, denominated “exemplary substitutions” in Table 3, or as further described below in reference to amino acid classes, may be introduced and the products screened for a desired characteristic.

TABLE 3
Amino acid substitutions

| Original | Exemplary Substitutions | Preferred Substitutions |
|---|---|---|
| Ala (A) | val, leu, ile | val |
| Arg I | lys, gln, asn | lys |
| Asn (N) | gln, his, asp, lys, arg | gln |
| Asp (D) | glu, asn | glu |
| Cys I | ser, ala | ser |
| Gln (Q) | asn, glu | asn |
| Glu I | asp, gln | Asp |
| Gly (G) | Ala | Ala |
| His (H) | asn, gln, lys, arg | Arg |
| Ile (I) | leu, val, met, ala, phe | Leu |
| Leu (L) | norleucine, ile, val, met, ala | Ile |
| Lys (K) | arg, gln, asn | Arg |
| Met (M) | leu, phe, ile | Leu |
| Phe (F) | leu, val, ile, ala, tyr | Tyr |
| Pro (P) | Ala | Ala |
| Ser (S) | Thr | Thr |
| Thr (T) | Ser | Ser |
| Trp (W) | tyr, phe | Tyr |
| Tyr (Y) | trp, phe, thr, ser | Phe |
| Val (V) | ile, leu, met, phe, ala | Leu |

Substantial modifications in the biological properties of the antibody construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common sidechain properties: (1) hydrophobic: norleucine, met, ala, val, leu, ile; (2) neutral hydrophilic: cys, ser, thir, asn, gln; (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antibody construct may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs or VH/VL sequences are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antibody construct. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs or VH/VL sequences and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” or a “variant VH/VL region” is one with the specified homology, similarity, or identity to the parent CDR/VH/VL of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR or VH/VL.

In one embodiment, the percentage of identity to human germline of the antibody constructs according to the invention is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang & Foote (“Immunogenicity of engineered antibodies”; Methods 36 (2005) 3-10) demonstrate that the reduction of non-human portions of drug antibody constructs leads to a decrease of risk to induce anti-drug antibodies in the patients during treatment. By comparing an exhaustive number of clinically evaluated antibody drugs and the respective immunogenicity data, the trend is shown that humanization of the V-regions of antibodies makes the protein less immunogenic (average 5.1% of patients) than antibodies carrying unaltered non-human V regions (average 23.59% of patients). A higher degree of identity to human sequences is hence desirable for V-region based protein therapeutics in the form of antibody constructs. For this purpose of determining the germline identity, the V-regions of VL can be aligned with the amino acid sequences of human germline V segments and J segments (http://vbase.mrc-cpe.cam.ac.uk/) using Vector NTI software and the amino acid sequence calculated by dividing the identical amino acid residues by the total number of amino acid residues of the VL in percent. The same can be for the VH segments (http://vbase.mrc-cpe.cam.ac.uk/) with the exception that the VH CDR3 may be excluded due to its high diversity and a lack of existing human germline VH CDR3 alignment partners. Recombinant techniques can then be used to increase sequence identity to human antibody germline genes.

In a further embodiment, the bispecific antigen-binding polypeptides of the present invention exhibit high monomer yields under standard research scale conditions, e.g., in a standard two-step purification process. Preferably the monomer yield of the antigen-binding polypeptides according to the invention is ≥0.25 mg/L supernatant, more preferably ≥0.5 mg/L, even more preferably ≥1 mg/L, and most preferably ≥3 mg/L supernatant.

Likewise, the yield of the dimeric antigen-binding polypeptide isoforms and hence the monomer percentage (i.e., monomer: (monomer+dimer)) of the antigen-binding polypeptides can be determined. The productivity of monomeric and dimeric antigen-binding polypeptides and the calculated monomer percentage can e.g. be obtained in the SEC purification step of culture supernatant from standardized research-scale production in roller bottles. In one embodiment, the monomer percentage of the antigen-binding polypeptides is ≥80%, more preferably ≥85%, even more preferably ≥90%, and most preferably ≥95%.

In one embodiment, the antigen-binding polypeptides have a preferred plasma stability (ratio of EC50 with plasma to EC50 w/o plasma) of ≤5 or ≤4, more preferably ≤3.5 or ≤3, even more preferably ≤2.5 or ≤2, and most preferably ≤1.5 or ≤1. The plasma stability of an antigen-binding polypeptide can be tested by incubation of the construct in human plasma at 37° C. for 24 hours followed by EC50 determination in a ⁵¹chromium release cytotoxicity assay. The effector cells in the cytotoxicity assay can be stimulated enriched human CD8 positive T cells. Target cells can e.g. be CHO cells transfected with the human target cell surface antigen. The effector to target cell (E:T) ratio can be chosen as 10:1. The human plasma pool used for this purpose is derived from the blood of healthy donors collected by EDTA coated syringes. Cellular components are removed by centrifugation and the upper plasma phase is collected and subsequently pooled. As control, antigen-binding polypeptides are diluted immediately prior to the cytotoxicity assay in RPMI-1640 medium. The plasma stability is calculated as ratio of EC50 (after plasma incubation) to EC50 (control).

It is furthermore preferred that the monomer to dimer conversion of antigen-binding polypeptides of the invention is low. The conversion can be measured under different conditions and analyzed by high performance size exclusion chromatography. For example, incubation of the monomeric isoforms of the antigen-binding polypeptides can be carried out for 7 days at 37° C. and concentrations of e.g. 100 μg/ml or 250 μg/ml in an incubator. Under these conditions, it is preferred that the antigen-binding polypeptides of the invention show a dimer percentage that is ≤5%, more preferably ≤4%, even more preferably ≤3%, even more preferably ≤2.5%, even more preferably ≤2%, even more preferably ≤1.5%, and most preferably ≤1% or ≤0.5% or even 0%.

It is also preferred that the bispecific antigen-binding polypeptides of the present invention present with very low dimer conversion after a number of freeze/thaw cycles. For example, the antigen-binding polypeptide monomer is adjusted to a concentration of 250 μg/ml e.g. in generic formulation buffer and subjected to three freeze/thaw cycles (freezing at −80° C. for 30 min followed by thawing for 30 min at room temperature), followed by high performance SEC to determine the percentage of initially monomeric antigen-binding polypeptide, which had been converted into dimeric antigen-binding polypeptide. Preferably the dimer percentages of the bispecific antigen-binding polypeptides are 5%, more preferably ≤4%, even more preferably ≤3%, even more preferably ≤2.5%, even more preferably ≤2%, even more preferably ≤1.5%, and most preferably ≤1% or even ≤0.5%, for example after three freeze/thaw cycles.

The bispecific antigen-binding polypeptides of the present invention preferably show a favorable thermostability with aggregation temperatures ≥45° C. or ≥50° C., more preferably ≥52° C. or ≥54° C., even more preferably ≥56° C. or ≥57° C., and most preferably ≥58° C. or ≥59° C. The thermostability parameter can be determined in terms of antibody aggregation temperature as follows: Antibody solution at a concentration 250 μg/ml is transferred into a single use cuvette and placed in a Dynamic Light Scattering (DLS) device. The sample is heated from 40° C. to 70° C. at a heating rate of 0.5° C./min with constant acquisition of the measured radius. Increase of radius indicating melting of the protein and aggregation is used to calculate the aggregation temperature of the antibody.

Alternatively, temperature melting curves can be determined by Differential Scanning Calorimetry (DSC) to determine intrinsic biophysical protein stabilities of the antigen-binding polypeptides. These experiments are performed using a MicroCal LLC (Northampton, Mass., U.S.A) VP-DSC device. The energy uptake of a sample containing an antigen-binding polypeptide is recorded from 20° C. to 90° C. compared to a sample containing only the formulation buffer. The antigen-binding polypeptides are adjusted to a final concentration of 250 μg/ml e.g. in SEC running buffer. For recording of the respective melting curve, the overall sample temperature is increased stepwise. At each temperature T energy uptake of the sample and the formulation buffer reference is recorded. The difference in energy uptake Cp (kcal/mole/° C.) of the sample minus the reference is plotted against the respective temperature. The melting temperature is defined as the temperature at the first maximum of energy uptake.

The target cell surface antigen×CD3 bispecific antigen-binding polypeptides of the invention are also envisaged to have a turbidity (as measured by OD340 after concentration of purified monomeric antigen-binding polypeptide to 2.5 mg/ml and over night incubation) of ≤0.2, preferably of 0.15, more preferably of ≤0.12, even more preferably of ≤0.1, and most preferably of ≤0.08.

In a further embodiment the antigen-binding polypeptide according to the invention is stable at physiologic or slightly lower pH, i.e. about pH 7.4 to 6.0. The more tolerant the antigen-binding polypeptide behaves at unphysiologic pH such as about pH 6.0, the higher is the recovery of the antigen-binding polypeptide eluted from an ion exchange column relative to the total amount of loaded protein. Recovery of the antigen-binding polypeptide from an ion (e.g., cation) exchange column at about pH 6.0 is preferably ≥30%, more preferably ≥40%, more preferably ≥50%, even more preferably ≥60%, even more preferably ≥70%, even more preferably ≥80%, even more preferably ≥90%, even more preferably ≥95%, and most preferably ≥99%.

It is furthermore envisaged that the bispecific antigen-binding polypeptides of the present invention exhibit therapeutic efficacy or anti-tumor activity. This can e.g. be assessed in a study as disclosed in the following example of an advanced stage human tumor xenograft model:

The skilled person knows how to modify or adapt certain parameters of this study, such as the number of injected tumor cells, the site of injection, the number of transplanted human T cells, the amount of bispecific antigen-binding polypeptides to be administered, and the timelines, while still arriving at a meaningful and reproducible result. Preferably, the tumor growth inhibition T/C [%] is ≤70 or ≤60, more preferably ≤50 or ≤40, even more preferably ≤30 or ≤20 and most preferably ≤10 or ≤5 or even ≤2.5.

In a preferred embodiment of the antigen-binding polypeptide of the invention the antigen-binding polypeptide is a single chain antigen-binding polypeptide.

Also in a preferred embodiment of the antigen-binding polypeptide of the invention said third domain comprises in an amino to carboxyl order:
hinge-CH2-CH3-linker-hinge-CH2-CH3.

Also in one embodiment of the invention the CH2 domain of one or preferably each (both) polypeptide monomers of the third domain comprises an intra domain cysteine disulfide bridge. As known in the art the term “cysteine disulfide bridge” refers to a functional group with the general structure R—S—S—R. The linkage is also called an SS-bond or a disulfide bridge and is derived by the coupling of two thiol groups of cysteine residues. It is particularly preferred for the antigen-binding polypeptide of the invention that the cysteines forming the cysteine disulfide bridge in the mature antigen-binding polypeptide are introduced into the amino acid sequence of the CH2 domain corresponding to 309 and 321 (Kabat numbering).

In one embodiment of the invention a glycosylation site in Kabat position 314 of the CH2 domain is removed. It is preferred that this removal of the glycosylation site is achieved by a N314X substitution, wherein X is any amino acid excluding Q. Said substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).

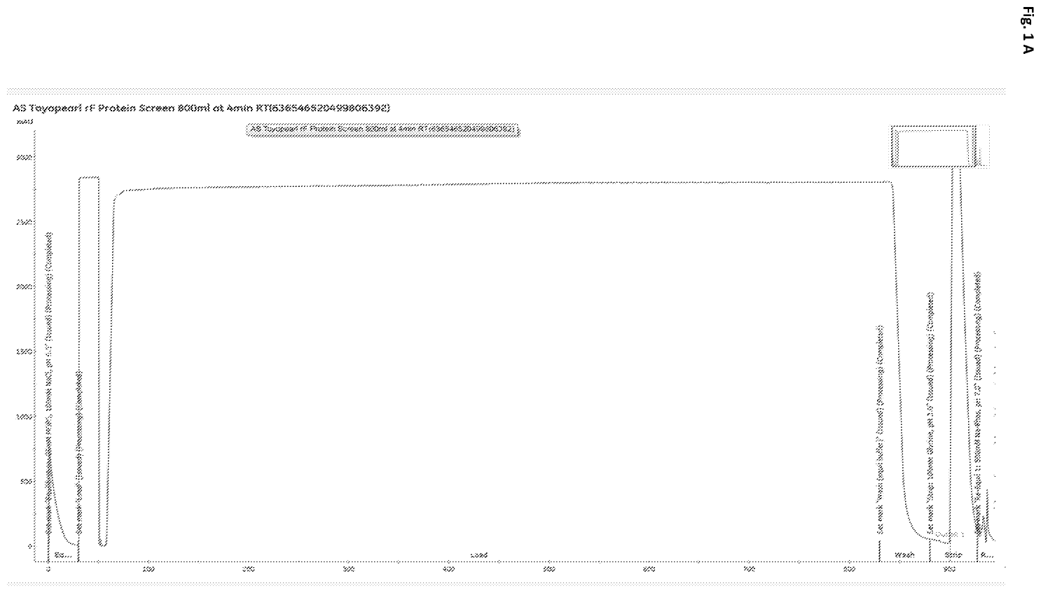

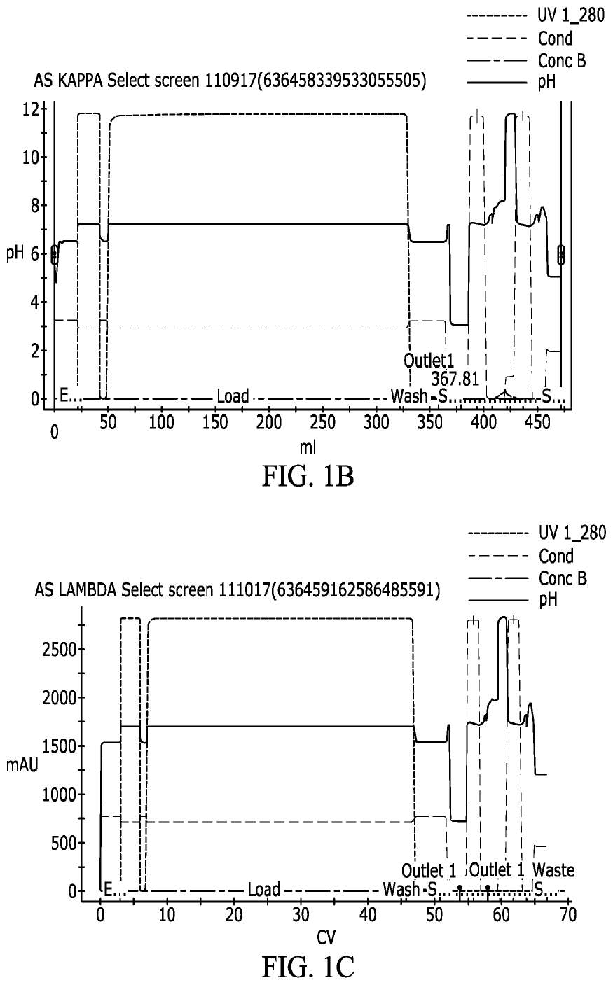

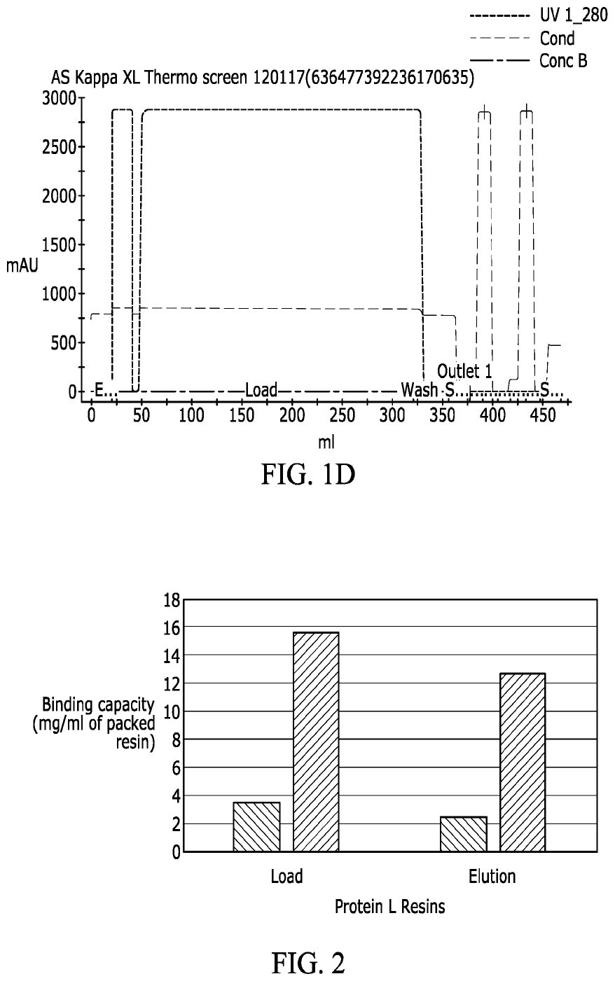

It is assumed that the preferred features of the antigen-binding polypeptide of the invention compared e.g. to the bispecific heteroFc antigen-binding polypeptide known in the art (FIG. 1b) may be inter alia related to the introduction of the above described modifications in the CH2 domain. Thus, it is preferred for the construct of the invention that the CH2 domains in the third domain of the antigen-binding polypeptide of the invention comprise the intra domain cysteine disulfide bridge at Kabat positions 309 and 321 and/or the glycosylation site at Kabat position 314 is removed by a N314X substitution as above, preferably by a N314G substitution.

In a further preferred embodiment of the invention the CH2 domains in the third domain of the antigen-binding polypeptide of the invention comprise the intra domain cysteine disulfide bridge at Kabat positions 309 and 321 and the glycosylation site at Kabat position 314 is removed by a N314G substitution.

In one embodiment the invention provides an antigen-binding polypeptide, wherein: (182) the first domain comprises two antibody variable domains and the second domain comprises two antibody variable domains;

(ii) the first domain comprises one antibody variable domain and the second domain comprises two antibody variable domains;
(iii) the first domain comprises two antibody variable domains and the second domain comprises one antibody variable domain; or
(iv) the first domain comprises one antibody variable domain and the second domain comprises one antibody variable domain.

Accordingly, the first and the second domain may be binding domains comprising each two antibody variable domains such as a VH and a VL domain. Examples for such binding domains comprising two antibody variable domains where described herein above and comprise e.g. Fv fragments, scFv fragments or Fab fragments described herein above. Alternatively either one or both of those binding domains may comprise only a single variable domain. Examples for such single domain binding domains where described herein above and comprise e.g. nanobodies or single variable domain antibodies comprising merely one variable domain, which might be VHH, VH or VL, that specifically bind an antigen or epitope independently of other V regions or domains.

In a preferred embodiment of the antigen-binding polypeptide of the invention first and second domain are fused to the third domain via a peptide linker. Preferred peptide linker have been described herein above and are characterized by the amino acid sequence Gly-Gly-Gly-Gly-Ser, i.e. Gly₄Ser (SEQ ID NO: 187), or polymers thereof, i.e. (Gly₄Ser)x, where x is an integer of 1 or greater (e.g. 2 or 3). A particularly preferred linker for the fusion of the first and second domain to the third domain is depicted in SEQ ID Nos: 1.

In a preferred embodiment the antigen-binding polypeptide of the invention is characterized to comprise in an amino to carboxyl order:

(a) the first domain;
(b) a peptide linker having an amino acid sequence selected from the group consisting of SEQ ID Nos: 187-189;
I the second domain;
(d) a peptide linker having an amino acid sequence selected from the group consisting of SEQ ID NO: 187, 188, 189, 195, 196, 197 and 198;
I the first polypeptide monomer of the third domain;
(f) a peptide linker having an amino acid sequence selected from the group consisting of SEQ ID Nos: 191, 192, 193 and 194; and
(g) the second polypeptide monomer of the third domain.

In one aspect of the invention the target cell surface antigen bound by the first domain is a tumor antigen, an antigen specific for an immunological disorder or a viral antigen. The term “tumor antigen” as used herein may be understood as those antigens that are presented on tumor cells. These antigens can be presented on the cell surface with an extracellular part, which is often combined with a transmembrane and cytoplasmic part of the molecule. These antigens can sometimes be presented only by tumor cells and never by the normal ones. Tumor antigens can be exclusively expressed on tumor cells or might represent a tumor specific mutation compared to normal cells. In this case, they are called tumor-specific antigens. More common are antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue. Non-limiting examples of tumor antigens as used herein are CDH19, MSLN, DLL3, FLT3, EGFRvIII, CD33, CD19, CD20, CD70, BCMA and PSMA.

Further target cell surface antigens specific for an immunological disorder in the context of the present invention comprise, for example, TL1A and TNF-alpha. Said targets are preferably addressed by a bispecific antigen-binding polypeptide of the present invention, which is preferably a full length antibody. In a very preferred embodiment, an antibody of the present invention is a hetero IgG antibody.

In a preferred embodiment of the antigen-binding polypeptide of the invention the tumor antigen is selected from the group consisting of CDH19, MSLN, DLL3, FLT3, EGFRvIII, CD33, CD19, CD20, CD70, BCMA and PSMA.

In one aspect of the invention the antigen-binding polypeptide comprises in an amino to carboxyl order:

(a) the first domain having an amino acid sequence selected from the group consisting of SEQ ID Nos: 7, 8, 17, 27, 28, 37, 38, 39, 40, 41, 48, 49, 50, 51, 52, 59, 60, 61, 62, 63, 64, 71, 72, 73, 74, 75. 76, 77, 78, 79, 80, 81, 89, 90, 91, 92, 93, 100, 101, 102, 103, 104, 113, 114, 121, 122,123, 124, 125, 131, 132, 133, 134, 135, 136, 143, 144, 145, 146, 147, 148, 149, 150, 151, 158, 159, 160, 161, 162, 163, 164, 165, 166, 173, 174, 175, 176, 177, 178, 179, 180, 181
(b) a peptide linker having an amino acid sequence selected from the group consisting of SEQ ID Nos: 187-189;
I the second domain having an amino acid sequence selected from the group consisting of SEQ ID Nos: SEQ ID Nos: 23, 25, 41, 43, 59, 61, 77, 79, 95, 97, 113, 115, 131, 133, 149, 151, 167, 169, 185 or 187 of WO 2008/119567 or of SEQ ID NO: 202;
(d) a peptide linker having an amino acid sequence selected from the group consisting of SEQ ID Nos: 187, 188, 189, 195, 196, 197 and 198;
I the first polypeptide monomer of the third domain having a polypeptide sequence selected from the group consisting of SEQ ID Nos: 17-24 of WO2017/134140;
(f) a peptide linker having an amino acid sequence selected from the group consisting of SEQ ID Nos: 191, 192, 193 and 194; and
(g) the second polypeptide monomer of the third domain having a polypeptide sequence selected from the group consisting of SEQ ID Nos: 17-24 of WO2017/134140.

In one aspect, the bispecific antigen-binding polypeptide of the invention is characterized by having an amino acid sequence selected from the group consisting of and being directed to the respective target cell surface antigen:

### (a) SEQ ID Nos: 27, 28, 37 to 41; CD33

(b) SEQ ID Nos: each of 48 to 52; EGFRvIII
(c) SEQ ID Nos: each of 59 to 64; MSLN
(d) SEQ ID Nos: each of 71 to 82 CDH19
(e) SEQ ID Nos: each of 100 to 104 DLL3

### (f) SEQ ID Nos: 7, 8, 17, 113 and 114CD19

(g) SEQ ID Nos: each of 89 to 93 FLT3
(h) SEQ ID Nos: each of 121 to 125 CDH3
(i) SEQ ID Nos: each of 132 to 136 BCMA and
(j) SEQ ID Nos: each of 143 to 151, 158 to 166 and 173 to 181 PSMA

The invention further provides a polynucleotide/nucleic acid molecule encoding an antigen-binding polypeptide of the invention. A polynucleotide is a biopolymer composed of 13 or more nucleotide monomers covalently bonded in a chain. DNA (such as cDNA) and RNA (such as mRNA) are examples of polynucleotides with distinct biological function. Nucleotides are organic molecules that serve as the monomers or subunits of nucleic acid molecules like DNA or RNA. The nucleic acid molecule or polynucleotide can be double stranded and single stranded, linear and circular. It is preferably comprised in a vector which is preferably comprised in a host cell. Said host cell is, e.g. after transformation or transfection with the vector or the polynucleotide of the invention, capable of expressing the antigen-binding polypeptide. For that purpose the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the vast majority of genes are encoded with exactly the same code, this particular code is often referred to as the canonical or standard genetic code. While the genetic code determines the protein sequence for a given coding region, other genomic regions can influence when and where these proteins are produced.

Furthermore, the invention provides a vector comprising a polynucleotide/nucleic acid molecule of the invention. A vector is a nucleic acid molecule used as a vehicle to transfer (foreign) genetic material into a cell. The term “vector” encompasses—but is not restricted to—plasmids, viruses, cosmids and artificial chromosomes. In general, engineered vectors comprise an origin of replication, a multicloning site and a selectable marker. The vector itself is generally a nucleotide sequence, commonly a DNA sequence that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding side. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding side is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using calcium phosphate, by electroporation, by cell squeezing or by mixing a cationic lipid with the material to produce liposomes, which fuse with the cell membrane and deposit their cargo inside.

The term “transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be effected by artificial means. For transformation to happen, cells or bacteria must be in a state of competence, which might occur as a time-limited response to environmental conditions such as starvation and cell density.

Moreover, the invention provides a host cell transformed or transfected with the polynucleotide/nucleic acid molecule or with the vector of the invention. As used herein, the terms “host cell” or “recipient cell” are intended to include any individual cell or cell culture that can be or has/have been recipients of vectors, exogenous nucleic acid molecules, and polynucleotides encoding the antigen-binding polypeptide of the present invention; and/or recipients of the antigen-binding polypeptide itself. The introduction of the respective material into the cell is carried out by way of transformation, transfection and the like. The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell, but is still included within the scope of the term as used herein. Suitable host cells include prokaryotic or eukaryotic cells, and also include but are not limited to bacteria, yeast cells, fungi cells, plant cells, and animal cells such as insect cells and mammalian cells, e.g., murine, rat, macaque or human.

The antigen-binding polypeptide of the invention can be produced in bacteria. After expression, the antigen-binding polypeptide of the invention is isolated from the E. coli cell paste in a soluble fraction and can be purified through, e.g., affinity chromatography and/or size exclusion. Final purification can be carried out similar to the process for purifying antibody expressed e.g., in CHO cells.

In addition to prokaryotes, eukaryotic microbes such as filamentous fungi or yeast are suitable cloning or expression hosts for the antigen-binding polypeptide of the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of glycosylated antigen-binding polypeptide of the invention are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36: 59 (1977)); baby hamster kidney cells (BHK, ATCC CCL 10); Chinese hamster ovary cells/-DHFR (CHO, Urlaub et al., Proc. Natl. Acad. Sci. USA 77: 4216 (1980)); mouse sertoli cells (TM4, Mather, Biol. Reprod. 23: 243-251 (1980)); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, 1413 8065); mouse mammary tumor (MMT 060562, ATCC CCL5 1); TRI cells (Mather et al., Annals N. Y Acad. Sci. (1982) 383: 44-68); MRC 5 cells; FS4 cells; and a human hepatoma line (Hep G2).

In a further embodiment the invention provides a process for the production of an antigen-binding polypeptide of the invention, said process comprising culturing a host cell of the invention under conditions allowing the expression of the antigen-binding polypeptide of the invention and recovering the produced antigen-binding polypeptide from the culture.

As used herein, the term “culturing” refers to the in vitro maintenance, differentiation, growth, proliferation and/or propagation of cells under suitable conditions in a medium. The term “expression” includes any step involved in the production of an antigen-binding polypeptide of the invention including, but not limited to, transcription, post-transcriptional modification, translation, post-translational modification, and secretion.

When using recombinant techniques, the antigen-binding polypeptide can be produced intracellularly, in the periplasmic space, or directly secreted into the medium. If the antigen-binding polypeptide is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antigen-binding polypeptide of the invention prepared from the host cells can be recovered or purified using, for example, hydroxylapatite chromatography, gel electrophoresis, dialysis, and affinity chromatography. Other techniques for protein purification such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), 59yophili-focusing, SDS-PAGE, and ammonium sulfate precipitation are also available depending on the antibody to be recovered. Where the antigen-binding polypeptide of the invention comprises a CH3 domain, the Bakerbond ABX resin (J. T. Baker, Phillipsburg, N.J.) is useful for purification.

Affinity chromatography is a preferred purification technique. The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose.

Moreover, the invention provides a pharmaceutical composition comprising an antigen-binding polypeptide of the invention or an antigen-binding polypeptide produced according to the process of the invention. It is preferred for the pharmaceutical composition of the invention that the homogeneity of the antigen-binding polypeptide is ≥80%, more preferably ≥81%, ≥82%, ≥83%, ≥84%, or ≥85%, further preferably ≥86%, ≥87%, ≥88%, ≥89%, or ≥90%, still further preferably, ≥91%, ≥92%, ≥93%, ≥94%, or ≥95% and most preferably ≥96%, ≥97%, ≥98% or ≥99%.

As used herein, the term “pharmaceutical composition” relates to a composition which is suitable for administration to a patient, preferably a human patient. The particularly preferred pharmaceutical composition of this invention comprises one or a plurality of the antigen-binding polypeptide(s) of the invention, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions of the invention include, but are not limited to, liquid, frozen, and lyophilized compositions.

The inventive compositions may comprise a pharmaceutically acceptable carrier. In general, as used herein, “pharmaceutically acceptable carrier” means any and all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the antigen-binding polypeptide of the invention and further one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of the invention to improve effectiveness and or to stabilize such formulations and processes against degradation and spoilage due to, for instance, stresses that occur during manufacturing, shipping, storage, pre-use preparation, administration, and thereafter.

In certain embodiments, the pharmaceutical composition may contain formulation materials for the purpose of modifying, maintaining or preserving, e.g., the pH, osmolarity, viscosity, clarity, color, isotonicity, odor, sterility, stability, rate of dissolution or release, adsorption or penetration of the composition (see, REMINGTON'S PHARMACEUTICAL SCIENCES, 18″ Edition, (A. R. Genrmo, ed.), 1990, Mack Publishing Company). In such embodiments, suitable formulation materials may include, but are not limited to:
amino acids such as glycine, alanine, glutamine, asparagine, threonine, proline, 2-phenylalanine, including charged amino acids, preferably lysine, lysine acetate, arginine, glutamate and/or histidine
antimicrobials such as antibacterial and antifungal agents
antioxidants such as ascorbic acid, methionine, or sodium hydrogen-sulfite;
buffers, buffer systems and buffering agents which are used to maintain the composition at physiological pH or at a slightly lower pH; examples of buffers are borate, bicarbonate, Tris-HCl, citrates, phosphates or other organic acids, succinate, phosphate, and histidine; for example Tris buffer of about pH 7.0-8.5;
non-aqueous solvents such as propylene glycol, polyethylene glycol, vegetable oils such as olive oil, and injectable organic esters such as ethyl oleate;
aqueous carriers including water, alcoholic/aqueous solutions, emulsions or suspensions, including saline and buffered media;
biodegradable polymers such as polyesters;
bulking agents such as mannitol or glycine;
chelating agents such as ethylenediamine tetraacetic acid (EDTA);
isotonic and absorption delaying agents;
complexing agents such as caffeine, polyvinylpyrrolidone, beta-cyclodextrin or hydroxypropyl-beta-cyclodextrin)
fillers;
monosaccharides; disaccharides; and other carbohydrates (such as glucose, mannose or dextrins); carbohydrates may be non-reducing sugars, preferably trehalose, sucrose, octasulfate, sorbitol or xylitol;
(low molecular weight) proteins, polypeptides or proteinaceous carriers such as human or bovine serum albumin, gelatin or immunoglobulins, preferably of human origin;
coloring and flavouring agents;
sulfur containing reducing agents, such as glutathione, thioctic acid, sodium thioglycolate, thioglycerol, [alpha]-monothioglycerol, and sodium thio sulfate
diluting agents;
emulsifying agents;
hydrophilic polymers such as polyvinylpyrrolidone)
salt-forming counter-ions such as sodium;
preservatives such as antimicrobials, anti-oxidants, chelating agents, inert gases and the like; examples are: benzalkonium chloride, benzoic acid, salicylic acid, thimerosal, phenethyl alcohol, methylparaben, propylparaben, chlorhexidine, sorbic acid or hydrogen peroxide);
metal complexes such as Zn-protein complexes;
solvents and co-solvents (such as, propylene glycol or polyethylene glycol);
sugars and sugar alcohols, such as trehalose, sucrose, octasulfate, mannitol, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; and polyhydric sugar alcohols;
suspending agents;
surfactants or wetting agents such as pluronics, PEG, sorbitan esters, polysorbates such as polysorbate 20, polysorbate, triton, tromethamine, lecithin, cholesterol, tyloxapol; surfactants may be detergents, preferably with a molecular weight of >1.2 KD and/or a polyether, preferably with a molecular weight of >3 KD; non-limiting examples for preferred detergents are Tween 20, Tween 40, Tween 60, Tween 80 and Tween 85; non-limiting examples for preferred polyethers are PEG 3000, PEG 3350, PEG 4000 and PEG 5000;
stability enhancing agents such as sucrose or sorbitol;
tonicity enhancing agents such as alkali metal halides, preferably sodium or potassium chloride, mannitol sorbitol;
parenteral delivery vehicles including sodium chloride solution, Ringer's dextrose, dextrose and sodium chloride, lactated Ringer's, or fixed oils;
intravenous delivery vehicles including fluid and nutrient replenishers, electrolyte replenishers (such as those based on Ringer's dextrose).

It is evident to those skilled in the art that the different constituents of the pharmaceutical composition (e.g., those listed above) can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

In a preferred aspect of the invention the pharmaceutical composition is stable for at least four weeks at about −20° C. As apparent from the appended examples the quality of an antibody construct of the invention vs. the quality of corresponding state of the art antibody constructs may be tested using different systems. Those tests are understood to be in line with the “ICH Harmonised Tripartite Guideline: Stability Testing of Biotechnological/Biological Products Q5C and Specifications: Test procedures and Acceptance Criteria for Biotech Biotechnological/Biological Products Q6B” and, thus are elected to provide a stability-indicating profile that provides certainty that changes in the identity, purity and potency of the product are detected. It is well accepted that the term purity is a relative term. Due to the effect of glycosylation, deamidation, or other heterogeneities, the absolute purity of a biotechnological/biological product should be typically assessed by more than one method and the purity value derived is method-dependent. For the purpose of stability testing, tests for purity should focus on methods for determination of degradation products.

For the assessment of the quality of a pharmaceutical composition comprising an antibody construct of the invention may be analyzed e.g. by analyzing the content of soluble aggregates in a solution (HMWS per size exclusion). It is preferred that stability for at least four weeks at about −20° C. is characterized by a content of less than 1.5% HMWS, preferably by less than 1% HMWS.

A preferred Product Quality Analytical Method herein is Size Exclusion-High Performance Liquid Chromatography (SE-HPLC). SE-HPLC is typically performed using a size exclusion column and an UHPLC system, e.g. Waters BEH200 size exclusion column (4.6×150 mm, 1.7 μm) and Waters UHPLC system. The protein samples are injected neat and separated isocratically using a phosphate buffer e.g. containing NaCl salt (mobile phase was 100 mM sodium phosphate, 250 mM NaCl at pH 6.8) at a flow rate of e.g. 0.4 m/min, and the eluent was monitored by UV absorbance at 280 nm. Typically, about 6 μg of sample is loaded.

Tryptic Peptide Mapping for Chemical Modifications Bispecific antibody construct protein samples are digested with a filter-based method using e.g. Millipore Microcon 30K device. The protein sample is added on the filter, centrifuged to remove the sample matrix, then denatured in e.g. 6M guanidine hydrochloride (GuHCl) (e.g. Thermo Fisher Scientific, Rockford, Ill.) buffer containing methionine, reduced with e.g. 500 mM dithiothreitol (DTT) (e.g. Sigma-Aldrich, St. Louis, Mo.) at e.g. 37° C. for 30 min, and subsequently alkylated by incubation with e.g. 500 mM iodoacetic acid (IAA) (e.g. Sigma-Aldrich, St. Louis, Mo.) for e.g. 20 min in the dark at room temperature. Unreacted IAA is quenched by adding DTT. All the above steps occurred on the filter. Samples are subsequently buffer exchanged into the digestion buffer (e.g. 50 mM Tris, pH 7.8 containing Methionine) by centrifuging to remove any residual DTT and IAA. Trypsin digestion is performed on the filter e.g. for 1 hr at 37° C. using an enzyme to protein ratio of 1:20 (w/w). The digestion mixture is collected by centrifuging and then quenched e.g. by adding 8M GuHCl in acetate buffer at pH 4.7.

The liquid chromatography-mass spectrometry (LC-MS) analysis is performed using a ultra-performance liquid chromatography (UPLC) system, e.g. Thermo U-3000, directly coupled with a Mass Spectrometer, e.g. Thermo Scientific Q-Exactive. The protein digests were separated by reversed phase using an Agilent Zorbax C18 RR HD column (2.1×150 mm, 1.8 μm), with the column temperature maintained at 50° C. The mobile phase A consisted of 0.020% (v/v) formic acid (FA) in water, and the mobile phase B was 0.018% (v/v) FA in acetonitrile (I). Approximately 5 μg of the digested bispecific antibody construct is injected to the column. A gradient (e.g. 0.5 to 36% B over 145 min) is used to separate the peptides at a flow rate, e.g. of 0.2 m/min. The eluted peptides are monitored by MS.

For peptide identification and modification analysis, a data-dependent tandem MS (MS/MS) experiment is typically utilized. A full scan is typically acquired, e.g. from 200 to 2000 m/z in the positive ion mode followed by e.g. 6 data-dependent MS/MS scans to identify the sequence of the peptide. The quantitation is based on mass spectrometry data of the selected ion monitoring using the equation below:

$$Modification \% = \frac{{A}_{modified}}{{A}_{modified} + {A}_{unmodified}} \times 100$$

Where Modification % is the level of the modified peptides, A_{modified} is the extracted ion chromatogram area of modified peptide, A_{unmodified} is the extracted ion chromatogram area of unmodified peptide.

Host Cell Protein (HCP) ELISA

A microtiter plate is coated with rabbit anti-HCP Immunoglobulin G (IgG) (Amgen, in-house antibody). After the plate is washed and blocked, the test samples, controls and HCP calibration standards are added to the plate and incubated. Unbound proteins are washed from the plate and pooled rabbit anti-HCP IgG-Biotin (Amgen, in-house antibody) is added to the plate and incubated. Following another wash, Streptavidin™ Horseradish Peroxidase conjugate (HRP-conjugate) (e.g. Amersham—GE, Buckinghamshire, UK) is added to the plate and incubated. The plate is washed a final time and the chromogenic substrate tetramethylbenzidine (TMB) (e.g. Kirkegaard and Perry Laboratories, Gaithersburg, Md.) is added to plate. Color development is arrested with 1 M Phosphoric acid and the optical density is measured with a spectrophotometer.

Other examples for the assessment of the stability of an antigen-binding polypeptide of the invention in form of a pharmaceutical composition are provided in the appended examples 4-12. In those examples embodiments of antigen-binding polypeptides of the invention are tested with respect to different stress conditions in different pharmaceutical formulations and the results compared with other half-life extending (HLE) formats of bispecific T cell engaging antigen-binding polypeptide known from the art. In general, it is envisaged that antigen-binding polypeptides provided with the specific FC modality according to the present invention are typically more stable over a broad range of stress conditions such as temperature and light stress, both compared to antigen-binding polypeptides provided with different HLE formats and without any HLE format (e.g. “canonical” antigen-binding polypeptides). Said temperature stability may relate both to decreased (below room temperature including freezing) and increased (above room temperature including temperatures up to or above body temperature) temperature. As the person skilled in the art will acknowledge, such improved stability with regard to stress, which is hardly avoidable in clinical practice, makes the antigen-binding polypeptide safer because less degradation products will occur in clinical practice. In consequence, said increased stability means increased safety.

One embodiment provides the antigen-binding polypeptide of the invention or the antigen-binding polypeptide produced according to the process of the invention for use in the prevention, treatment or amelioration of a proliferative disease, a tumorous disease, a viral disease or an immunological disorder.

The formulations described herein are useful as pharmaceutical compositions in the treatment, amelioration and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement of the disease state of a patient having a tumor or cancer or a metastatic cancer as specified herein below, by the administration of an antigen-binding polypeptide according to the invention to a subject in need thereof. Such an improvement may also be seen as a slowing or stopping of the progression of the tumor or cancer or metastatic cancer of the patient. The term “prevention” as used herein means the avoidance of the occurrence or re-occurrence of a patient having a tumor or cancer or a metastatic cancer as specified herein below, by the administration of an antigen-binding polypeptide according to the invention to a subject in need thereof.

The term “disease” refers to any condition that would benefit from treatment with the antibody construct or the pharmaceutic composition described herein. This includes chronic and acute disorders or diseases including those pathological conditions that predispose the mammal to the disease in question.

A “neoplasm” is an abnormal growth of tissue, usually but not always forming a mass. When also forming a mass, it is commonly referred to as a “tumor”. Neoplasms or tumors or can be benign, potentially malignant (pre-cancerous), or malignant. Malignant neoplasms are commonly called cancer. They usually invade and destroy the surrounding tissue and may form metastases, i.e., they spread to other parts, tissues or organs of the body. Hence, the term “metatstatic cancer” encompasses metastases to other tissues or organs than the one of the original tumor. Lymphomas and leukemias are lymphoid neoplasms. For the purposes of the present invention, they are also encompassed by the terms “tumor” or “cancer”.

The term “immunological disorder” as used herein describes in line with the common definition of this term immunological disorders such as autoimmune diseases, hypersensitivities, immune deficiencies.

In one embodiment the invention provides a method for the treatment or amelioration of a proliferative disease, a tumorous disease, a viral disease or an immunological disorder, comprising the step of administering to a subject in need thereof the antigen-binding polypeptide of the invention, or produced according to the process of the invention.

The terms “subject in need” or those “in need of treatment” includes those already with the disorder, as well as those in which the disorder is to be prevented. The subject in need or “patient” includes human and other mammalian subjects that receive either prophylactic or therapeutic treatment.

The antigen-binding polypeptide of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended to limit the scope of the present invention in any way.

### Example 1: Evaluation of CD33×CD3 Bispecific Antigen-Binding Polypeptide Chromatographic Capture Employing TOYOPEARL® AF-rProtein L-650F in Comparison to Capto® L

a) Column details
Two pre-packed columns [Lot #65PLFC501A, Part #0045162, Serial #00023 and 00042] were used. Columns were 8 mm ID and 10 cm in bed height. Each column was 5 mL in capacity.
b) Resin details
Two pre-packed column with TOYOPEARL AF-rProtein L-650F resins were used.
c) Pre-packed columns were used.
d) Feed conditions
The frozen feed solution was thawed in water bath at 25° C., either on the day of the test or a day before [kept in 2-8° C. overnight]. Once the feed solution was at room temperature, it was sterile filtered and used in the studies.
Example 1 results: CD33×CD3 bispecific antigen-binding polypeptide
Two pre-packed columns with mi TOYOPEARL AF-rProtein L-650F resin in each, were connected together to achieve a total bed height of 20 cm representing the bed height at pilot scale. The dynamic binding capacity study was performed by running load material as per the conditions shown in Table 5. Elution binding capacity achieved was 12.7 g/L-packed resin, which is a four factor improvement over the current affinity resin. The overall yield was in the similar range as per the current process. Several other benefits are possible with the four factor improvement in binding capacity. Using the new TOYOPEARL AF-rProtein L-650F with the four factor improved binding capacity would lead to six factor reduction in the number of cycles for volume reduction of harvested cell culture fluid [assuming a 5K L feed solution, Table 5]. These are significant benefits on the manufacturing scale [Table 5].

TABLE 4
Study Comparison details between pilot scale and small scale
Protein L resins
STUDY COMPARISON DETAILS

| | | TOYOPEARL ® |
|---|---|---|
| | | AF-rProtein |
| Resin | Capto L | L-650F |
| Study scale comparison | Pilot | Small |
| Column bed Height (cm) | 20 | 20 |
| Column ID (cm) | 10 | 0.8 |
| Column Volume (mL) | 1571 | 10 |
| Target Loading (g/L of packed resin) | 3.5 | 15.6 |
| Protein residence time (min) | 5.2 | 4 |
| Elution binding capacity/ml of packed resin | 2.1-3.4 | 12.7 |
| Overall Yield | 73-91% | 81% |
| Enhancement in Target/Elution Capacity | | 4× |

TABLE 5
Large scale specific benefit with use of TOYOPEARL ® AF-rProtein
L-650F resin
AT SCALE BENEFITS

| | | FUTURE |
|---|---|---|
| | | RESIN - |
| | | TOYOPEARL ® |
| | | AF-rProtein |
| Resin | Capto L | L-650F |
| Scale comparison | Large | Large |
| | Scale | Scale |
| Column bed Height (cm) | 20 | 20 |
| Column ID (cm) | 45 | 45 |
| Column Volume (mL) | 31800 | 31800 |
| Target Loading (g/L of packed resin) | 2.5 | 15.6 |
| # of cycles needed to process 5000 L HCCF | 12.6 | 2 |
| [Titer at 0.2 mg/mL] | | |
| Overall reduction in cycles | | 6× |
| With 20 cm ID column (2× reduction in | | 10 |
| column ID) # of cycles needed | | |

### Example 2: Evaluation of CD19×CD3 Bispecific Antigen-Binding Polypeptide Chromatographic Capture Employing TOYOPEARL® AF-rProtein L-650F in Comparison to Capto® L

a) Column details
Only one pre-packed column with similar details as above was used.
b) Resin details
One pre-packed column with TOYOPEARL AF-rProtein L-650F resin was used.
c) Pre-packed columns were used.
d) Feed conditions
The frozen feed solution was thawed in water bath at 25° C., either on the day of the test or a day before [kept in 2-8° C. overnight]. Once the feed solution was at room temperature, it was sterile filtered and used in the studies.
Example 2 results: CD19×CD3 bispecific antigen-binding polypeptide
One pre-packed column with 5 ml TOYOPEARL AF-rProtein L-650F resin, and 10 cm bed height representing the bed height at pilot scale was used for binding capacity measurements. The binding capacity study was performed by running load material as per the conditions shown in Table 6. Elution binding capacity achieved was comparable to the current affinity resin, but there is a potential to achieve a two factor improvement over the current affinity resin. Several other benefits are possible with the two factor improvement in binding capacity. Using the new TOYOPEARL AF-rProtein L-650F with the two factor improved binding capacity would lead to two factor reduction in the number of cycles for volume reduction of harvested cell culture fluid [assuming a 1K L feed solution, Table 7]. These are significant benefits on the manufacturing scale [Table 7]. Table 8 shows the product quality comparison between the screening runs and large scale GMP runs performed with the current Capto L resin.

TABLE 6
Study Comparison details between pilot scale and small scale
Protein L resins
STUDY COMPARISON DETAILS

| | | TOYOPEARL ® |
|---|---|---|
| | | AF-rProtein |
| Resin | Capto L | L-650F |
| Scale | Pilot | Small |
| Column bed Height (cm) | 13 | 10 |
| Column ID (cm) | 10 | 0.8 |
| Column Volume (mL) | 1020 | 5 |
| Target Loading | 8-9 | 11 |
| (g/L of packed resin) | | |
| Protein residence time (min) | 3 | 2.5 |
| Elution binding capacity | N/A | 9 |
| (g/L of packed resin) | | |
| Overall Yield | 80-100% | 77% |
| Potential Enhancement in | | 2× [Potential loading |
| Target/Elution Capacity | | to 18 mg/ml possible] |

TABLE 7
Large scale specific benefit with use of TOYOPEARL® AF-rProtein
L-650F resin
AT SCALE BENEFITS

| | | FUTURE |
|---|---|---|
| | | RESIN - |
| | | TOYOPEARL ® |
| | | AF-rProtein |
| Resin | Capto L | L-650F |
| Scale comparison | Large Scale | Large Scale |
| Column bed Height (cm) | 13 | 13 |
| Column ID (cm) | 10 | 10 |
| Column Volume (mL) | 1020 | 1020 |
| Target Loading (g/L of packed resin) | 8 | 18* |
| # of cycles needed to process 1000 L | 12 | 5 |
| HCCF [Titer at 0.1 mg/mL] | | |
| Overall reduction in cycles | | 2× |

*Loading at high target binding as per the potential highlighted in Table 6

TABLE 8
bispecific CD19 × CD3 antigen-binding polypeptide CM PQ results
comparison between Toyopearl Screen and GMP Runs
CD19 × CD3 bispecific construct PQ Results comparison to GMP Runs

| | | GMP Runs |
|---|---|---|
| | Toyopearl | (I and II) |
| | Screen Run I | (TS004403) |
| CEX- Acidic Peak % | 11.5 | 3.6-5.4 |
| CEX- Basic Peak % | 33.5 | 49.0-61.7 |
| CEX- Main Peak % | 55 | 34.3-45.6 |
| SE-HPLC [Monomer %] | 53.3 | 38.8-45.7 |
| SE-HPLC [Total Aggregate %] | 46.7 | 54.3-61.2 |
| SDS PAGE (MAIN BAND %) | 100 | 100 |
| DNA [pg/mg] | <17.6 | <10-<20 |

### Example 3: Evaluation of BCMA×CD3 Bispecific Antigen-Binding Polypeptide Chromatographic Capture Employing TOYOPEARL® AF-rProtein L-650F in Comparison to Capto® L

a) Column details
One Omnifit glass bore column with 6 mm ID, manually packed to 5 cm bed height was used.
b) Resin details
A 100 ml bottle of TOYOPEARL AF-rProtein L-650F resin [Lot #65PLFC03C] was used to manually pack an Omnifit 6 mm ID column.
c) Column packing
For Example 3, desired amount of TOYOPEARL AF-rProtein L-650F resin was suspended in a graduated cylinder to calculate slurry percentage in the shipped buffer for the resin. A calculated amount of the resin based on particular compression factor was then transferred into a 6 mm ID Omnifit glass bore column. The resin was then subsequently flow packed in 100 mM solution of sodium chloride, to the final target bed height of 5 cm
d) Feed conditions
The frozen feed solution was thawed in water bath at 25° C., either on the day of the test or a day before [kept in 2-8° C. overnight]. Once the feed solution was at room temperature, it was sterile filtered and used in the studies.
Example 3 results: BCMA×CD3 bispecific antigen-binding polypeptide
For the study three, a 6.6 mm ID glass bore Omnifit column was manually packed to perform the binding capacity measurements as per the conditions shown in Table 9. Elution binding capacity achieved was comparable to the current affinity resin, but there are significant time savings in load processing at the pilot scale, of approximately three hours [Table 9].

TABLE 9
Study Comparison details between pilot scale and small scale
Protein L resins
STUDY COMPARISON DETAILS

| | | TOYOPEARL ® |
|---|---|---|
| | Capto | AF-rProtein |
| Resin | L | L-650F |
| Scale | Pilot | Small |
| Column bed Height (cm) | 20 | 5 |
| Column ID (cm) | 25.1 | 0.66 |
| Column Volume (mL) | 9900 | 1.71 |
| Target Loading (g/L of packed resin) | 7-18 | 7-19 |
| Protein residence time (min) | 5 | 3 |
| Elution binding capacity (g/L of packed resin) | N/A | 6 |
| Overall Yield | >85% | 33% |
| Time taken to load at max binding capacity at | 7 | 4 |
| current residence time [hours] | | |
| Time savings at scale [hours] | | 3 |
| Potential Enhancement in Target/Elution | | N/A |
| Capacity | | |

TABLE 10
Wash and Elution Buffers used for all the Studies

| | | |
|---|---|---|
| Step | Buffers used | Ranges |

CD33 × CD3 bispecific

| | | |
|---|---|---|
| EQ/Wash | 25 mM MOPS; | 0-30 mM MOPS, |
| | 100 mM NaCl pH 6.5 | 50-150 mM NaCl |
| Elution | 25 mM Tris; | 15-35 mM Tris, |
| | 500 mM L-Arginine pH 7.5 | 0.25-1M Arginine |
| Elution 1 | 100 mM Glycine pH 3.0 | 50-150 mM Glycine |

CD19 × CD3 bispecific construct

| | | |
|---|---|---|
| Wash 1 | 25 mM Tris, 100 mM NaCl, | 15-35 mM Tris |
| | pH 7.4 | |
| Wash 2 | 25 Mm Tris, 0.5M Arginine, | 0.25-1M Arginine |
| | pH 7.5 | |
| Elution 2 | 100 mM Acetate, pH 3.3 | 50-150 mM Acetate |

BCMA × CD3 bispecific construct

| | | |
|---|---|---|
| EQ/Wash 1 | PBS: 8.03 mM Sodium phosphate, | PBS 0-1×, pH 7.4 |
| | 1.47 mM Potassium phosphate, | |
| | 2.68 mM KCl, | |
| | 137 mM NaCl, pH 7.4 | |
| Wash 2 | 100 mM Tris, 1M NaCl, | |
| | 250 mM L-Arginine, pH 8.0 | |
| Wash 3 | 50 mM Sodium Acetate, | 40-60 mM Acetate, |
| | pH 5.5 | pH 4-6 |
| Elution | 50 mM Sodium Acetate, | Low pH 3-3.5, |
| | pH 3.3 | 50-100 mM |
| Strip Buffer | 1M Acetic acid | |
| Regeneration | 6M Urea | Typically use 0.1M |
| | | NaOH |

TABLE 11
Sequence table

| | | | | |
|---|---|---|---|---|
| 1. | CD19 VL CDR1 | artificial | aa | KASQSVDYDGDSYLN |
| 2. | CD19 VL CDR2 | artificial | aa | DASNLVS |
| 3. | CD19 VL CDR3 | artificial | aa | QQSTEDPWT |
| 4. | CD19 VH CDR1 | artificial | aa | SYWMN |
| 5. | CD19 VH CDR2 | artificial | aa | QIWPGDGDTNYNGKFKG |
| 6. | CD19 VH CDR3 | artificial | aa | RETTTVGRYYYAMDY |
| 7. | CD19 VL | artificial | aa | DIQLTQSPASLAVSLGQRATISCKASQSVDYDGDSYLN |
| | | | | WYQQIPGQPPKLLIYDASNLVSGIPPRFSGSGSGTDFT |
| | | | | LNIHPVEKVDAATYHCQQSTEDPWTFGGGTKLEIK |
| 8. | CD19 VH | artificial | aa | QVQLQQSGAELVRPGSSVKISCKASGYAFSSYWMNWVK |
| | | | | QRPGQGLEWIGQIWPGDGDTNYNGKFKGKATLTADESS |
| | | | | STAYMQLSSLASEDSAVYFCARRETTTVGRYYYAMDYW |
| | | | | GQGTTVTVSS |
| 9. | CD3 VH CDR1 | artificial | aa | RYTMH |
| 10. | CD3 VH CDR2 | artificial | aa | YINPSRGYTNYNQKFKD |
| 11. | CD3 VH CDR3 | artificial | aa | YYDDHYCLDY |
| 12. | CD3 VL CDR1 | artificial | aa | RASSSVSYMN |
| 13. | CD3 VL CDR2 | artificial | aa | DTSKVAS |
| 14. | CD3 VL CDR3 | artificial | aa | QQWSSNPLT |
| 15. | CD3 VH | artificial | aa | DIKLQQSGAELARPGASVKMSCKTSGYTFTRYTMHWVK |
| | | | | QRPGQGL |
| | | | | EWIGYINPSRGYTNYNQKFKDKATLTTDKSSSTAYMQL |
| | | | | SSLTSEDSAVYYCARYYDDHYCLDYWGQGTTLTVSS |
| 16. | CD3 VL | artificial | aa | VDDIQLTQSPAIMSASPGEKVTMTCRASSSVSYMNWYQ |
| | | | | QKSGTSPKRWIYDTSKVASGVPYRFSGSGSGTSYSLTI |
| | | | | SSMEAEDAATYYCQQWSSNPLTFGAGTKLELK |
| 17. | CD19 × CD3 scFv | artificial | aa | DIQLTQSPASLAVSLGQRATISCKASQSVDYDGDSYLN |
| | BLINCYTO incl | | | WYQQIPGQPPKLLIYDASNLVSGIPPRFSGSGSGTDFT |
| | linker and his- | | | LNIHPVEKVDAATYHCQQSTEDPWTFGGGTKLEIKGGG |
| | tag | | | GSGGGGSGGGGSQVQLQQSGAELVRPGSSVKISCKASG |
| | | | | YAFSSYWMNWVKQRPGQGLEWIGQIWPGDGDTNYNGKF |
| | | | | KGKATLTADESSSTAYMQLSSLASEDSAVYFCARRETT |
| | | | | TVGRYYYAMDYWGQGTTVTVSSGGGGSDIKLQQSGAEL |
| | | | | ARPGASVKMSCKTSGYTFTRYTMHWVKQRPGQGLEWIG |
| | | | | YINPSRGYTNYNQKFKDKATLTTDKSSSTAYMQLSSLT |
| | | | | SEDSAVYYCARYYDDHYCLDYWGQGTTLTVSSVEGGSG |
| | | | | GSGGSGGSGGVDDIQLTQSPAIMSASPGEKVTMTCRAS |
| | | | | SSVSYMNWYQQKSGTSPKRWIYDTSKVASGVPYRFSGS |
| | | | | GSGTSYSLTISSMEAEDAATYYCQQWSSNPLTFGAGTK |
| | | | | LELKHHHHHH |
| 18. | CDR-L1 of I2C | artificial | aa | GSSTGAVTSGNYPN |
| 19. | CDR-L2 of I2C | artificial | aa | GTKFLAP |
| 20. | CDR-L3 of I2C | artificial | aa | VLWYSNRWV |
| 21. | CDR-H1 of I2C | artificial | aa | KYAMN |
| 22. | CDR-H2 of I2C | artificial | aa | RIRSKYNNYATYYADSVKD |
| 23. | CDR-H3 of I2C | artificial | aa | HGNFGNSYISYWAY |
| 24. | VH of I2C | artificial | aa | EVQLVESGGGLVQPGGSLKLSCAASGFTFNKYAMNWVR |
| | | | | QAPGKGLEWVARIRSKYNNYATYYADSVKDRFTISRDD |
| | | | | SKNTAYLQMNNLKTEDTAVYYCVRHGNFGNSYISYWAY |
| | | | | WGQGTLVTVSS |
| 25. | VL of I2C | artificial | aa | QTVVTQEPSLTVSPGGTVTLTCGSSTGAVTSGNYPNWV |
| | | | | QQKPGQAPRGLIGGTKFLAPGTPARFSGSLLGGKAALT |
| | | | | LSGVQPEDEAEYYCVLWYSNRWVFGGGTKLTVL |
| 26. | VH-VL of I2C | artificial | aa | EVQLVESGGGLVQPGGSLKLSCAASGFTFNKYAMNWVR |
| | | | | QAPGKGLEWVARIRSKYNNYATYYADSVKDRFTISRDD |
| | | | | SKNTAYLQMNNLKTEDTAVYYCVRHGNFGNSYISYWAY |
| | | | | WGQGTLVTVSSGGGGSGGGGSGGGGSQTVVTQEPSLTV |
| | | | | SPGGTVTLTCGSSTGAVTSGNYPNWVQQKPGQAPRGLI |
| | | | | GGTKFLAPGTPARFSGSLLGGKAALTLSGVQPEDEAEY |
| | | | | YCVLWYSNRWVFGGGTKLTVL |
| 27. | CD33 ccVH of | artificial | aa | QVQLVQSGAEVKKPGESVKVSCKASGYTFTNYGMNWVK |
| | E11 | | | QAPGQCLEWMGWINTYTGEPTYADKFQGRVTMTTDTST |
| | | | | STAYMEIRNLGGDDTAVYYCARWSWSDGYYVYFDYWGQ |
| | | | | GTSVTVSS |
| 28. | CD33 VH of E11 | Artificial | aa | QVQLVQSGAEVKKPGESVKVSCKASGYTFTNYGMNWVK |
| | | | | QAPGQGLEWMGWINTYTGEPTYADKFQGRVTMTTDTST |
| | | | | STAYMEIRNLGGDDTAVYYCARWSWSDGYYVYFDYWGQ |
| | | | | GTSVTVSS |
| 29. | CD33 HCDR1 of | artificial | aa | NYGMN |
| | E11 | | | |
| 30. | CD33 HCDR2 of | artificial | aa | WINTYTGEPTYADKFQG |
| | E11 | | | |
| 31. | CD33 HCDR3 of | artificial | aa | WSWSDGYYVYFDY |
| | E11 | | | |
| 32. | CD33 CC VL of | artificial | aa | DIVMTQSPDSLTVSLGERTTINCKSSQSVLDSSTNKNS |
| | E11 | | | LAWYQQKPGQPPKLLLSWASTRESGIPDRFSGSGSGTD |
| | | | | FTLTIDSPQPEDSATYYCQQSAHFPITFGCGTRLEIK |
| 33. | CD33 VL of E11 | artificial | aa | DIVMTQSPDSLTVSLGERTTINCKSSQSVLDSSTNKNS |
| | | | | LAWYQQKPGQPPKLLLSWASTRESGIPDRFSGSGSGTD |
| | | | | FTLTIDSPQPEDSATYYCQQSAHFPITFGQGTRLEIK |
| 34. | CD33 LCDR1 of | artificial | aa | KSSQSVLDSSTNKNSLA |
| | E11 | | | |
| 35. | CD33 LCDR2 of | artificial | aa | WASTRES |
| | E11 | | | |
| 36. | CD33 LCDR3 of | artificial | aa | QQSAHFPIT |
| | E11 | | | |
| 37. | CD33 HL CC of | artificial | aa | QVQLVQSGAEVKKPGESVKVSCKASGYTFTNYGMNWVK |
| | E11 | | | QAPGQCLEWMGWINTYTGEPTYADKFQGRVTMTTDTST |
| | | | | STAYMEIRNLGGDDTAVYYCARWSWSDGYYVYFDYWGQ |
| | | | | GTSVTVSSGGGGSGGGGSGGGGSDIVMTQSPDSLTVSL |
| | | | | GERTTINCKSSQSVLDSSTNKNSLAWYQQKPGQPPKLL |
| | | | | LSWASTRESGIPDRFSGSGSGTDFTLTIDSPQPEDSAT |
| | | | | YYCQQSAHFPITFGCGTRLEIK |
| 38. | CD33 HL of E11 | artificial | aa | QVQLVQSGAEVKKPGESVKVSCKASGYTFTNYGMNWVK |
| | | | | QAPGQGLEWMGWINTYTGEPTYADKFQGRVTMTTDTST |
| | | | | STAYMEIRNLGGDDTAVYYCARWSWSDGYYVYFDYWGQ |
| | | | | GTSVTVSSGGGGSGGGGSGGGGSDIVMTQSPDSLTVSL |
| | | | | GERTTINCKSSQSVLDSSTNKNSLAWYQQKPGQPPKLL |
| | | | | LSWASTRESGIPDRFSGSGSGTDFTLTIDSPQPEDSAT |
| | | | | YYCQQSAHFPITFGQGTRLEIK |
| 39. | CD33 CC E11 HL × | artificial | aa | QVQLVQSGAEVKKPGESVKVSCKASGYTFTNYGMNWVK |
| | I2C HL | | | QAPGQCLEWMGWINTYTGEPTYADKFQGRVTMTTDTST |
| | Bispecific | | | STAYMEIRNLGGDDTAVYYCARWSWSDGYYVYFDYWGQ |
| | molecule | | | GTSVTVSSGGGGSGGGGSGGGGSDIVMTQSPDSLTVSL |
| | | | | GERTTINCKSSQSVLDSSTNKNSLAWYQQKPGQPPKLL |
| | | | | LSWASTRESGIPDRFSGSGSGTDFTLTIDSPQPEDSAT |
| | | | | YYCQQSAHFPITFGCGTRLEIKSGGGGSEVQLVESGGG |
| | | | | LVQPGGSLKLSCAASGFTFNKYAMNWVRQAPGKGLEWV |
| | | | | ARIRSKYNNYATYYADSVKDRFTISRDDSKNTAYLQMN |
| | | | | NLKTEDTAVYYCVRHGNFGNSYISYWAYWGQGTLVTVS |
| | | | | SGGGGSGGGGSGGGGSQTVVTQEPSLTVSPGGTVTLTC |
| | | | | GSSTGAVTSGNYPNWVQQKPGQAPRGLIGGTKFLAPGT |
| | | | | PARFSGSLLGGKAALTLSGVQPEDEAEYYCVLWYSNRW |
| | | | | VFGGGTKLTVL |
| 40. | CD33 E11 HL × | artificial | aa | MGWSCIILFLVATATGVHSQVQLVQSGAEVKKPGESVK |
| | I2C HL | | | VSCKASGYTFTNYGMNWVKQAPGQGLEWMGWINTYTGE |
| | | | | PTYADKFQGRVTMTTDTSTSTAYMEIRNLGGDDTAVYY |
| | | | | CARWSWSDGYYVYFDYWGQGTSVTVSSGGGGSGGGGSG |
| | | | | GGGSDIVMTQSPDSLTVSLGERTTINCKSSQSVLDSST |
| | | | | NKNSLAWYQQKPGQPPKLLLSWASTRESGIPDRFSGSG |
| | | | | SGTDFTLTIDSPQPEDSATYYCQQSAHFPITFGQGTRL |
| | | | | EIKSGGGGSEVQLVESGGGLVQPGGSLKLSCAASGFTF |
| | | | | NKYAMNWVRQAPGKGLEWVARIRSKYNNYATYYADSVK |
| | | | | DRFTISRDDSKNTAYLQMNNLKTEDTAVYYCVRHGNFG |
| | | | | NSYISYWAYWGQGTLVTVSSGGGGSGGGGSGGGGSQTV |
| | | | | VTQEPSLTVSPGGTVTLTCGSSTGAVTSGNYPNWVQQK |
| | | | | PGQAPRGLIGGTKFLAPGTPARFSGSLLGGKAALTLSG |
| | | | | VQPEDEAEYYCVLWYSNRWVFGGGTKLTVLHHHHHH |
| 41. | CD33 CC × I2C- | artificial | aa | QVQLVQSGAEVKKPGESVKVSCKASGYTFTNYGMNWVK |
| | scFc Bispecific | | | QAPGQCLEWMGWINTYTGEPTYADKFQGRVTMTTDTST |
| | HLE molecule | | | STAYMEIRNLGGDDTAVYYCARWSWSDGYYVYFDYWGQ |
| | | | | GTSVTVSSGGGGSGGGGSGGGGSDIVMTQSPDSLTVSL |
| | | | | GERTTINCKSSQSVLDSSTNKNSLAWYQQKPGQPPKLL |
| | | | | LSWASTRESGIPDRFSGSGSGTDFTLTIDSPQPEDSAT |
| | | | | YYCQQSAHFPITFGCGTRLEIKSGGGGSEVQLVESGGG |
| | | | | LVQPGGSLKLSCAASGFTFNKYAMNWVRQAPGKGLEWV |
| | | | | ARIRSKYNNYATYYADSVKDRFTISRDDSKNTAYLQMN |
| | | | | NLKTEDTAVYYCVRHGNFGNSYISYWAYWGQGTLVTVS |
| | | | | SGGGGSGGGGSGGGGSQTVVTQEPSLTVSPGGTVTLTC |
| | | | | GSSTGAVTSGNYPNWVQQKPGQAPRGLIGGTKFLAPGT |
| | | | | PARFSGSLLGGKAALTLSGVQPEDEAEYYCVLWYSNRW |
| | | | | VFGGGTKLTVLGGGGDKTHTCPPCPAPELLGGPSVFLF |
| | | | | PPKPKDTLMISRTPEVTCVVVDVSHEDPEVKFNWYVDG |
| | | | | VEVHNAKTKPCEEQYGSTYRCVSVLTVLHQDWLNGKEY |
| | | | | KCKVSNKALPAPIEKTISKAKGQPREPQVYTLPPSREE |
| | | | | MTKNQVSLTCLVKGFYPSDIAVEWESNGQPENNYKTTP |
| | | | | PVLDSDGSFFLYSKLTVDKSRWQQGNVFSCSVMHEALH |
| | | | | NHYTQKSLSLSPGKGGGGSGGGGSGGGGSGGGGSGGGG |
| | | | | SGGGGSDKTHTCPPCPAPELLGGPSVFLFPPKPKDTLM |
| | | | | ISRTPEVTCVVVDVSHEDPEVKFNWYVDGVEVHNAKTK |
| | | | | PCEEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDGSF |
| | | | | FLYSKLTVDKSRWQQGNVFSCSVMHEALHNHYTQKSLS |
| | | | | LSPGK |
| 42. | EGFRvIII × CD3- | artificial | aa | NYGMH |
| | scFc VH CDR1 | | | |
| 43. | EGFRvIII × CD3- | artificial | aa | VIWYDGSDKYYADSVRG |
| | scFc VH CDR2 | | | |
| 44. | EGFRvIII × CD3- | artificial | aa | DGYDILTGNPRDFDY |
| | scFc VH CDR3 | | | |
| 45. | EGFRvIII × CD3- | artificial | aa | RSSQSLVHSDGNTYLS |
| | scFc VL CDR1 | | | |
| 46. | EGFRvIII × CD3- | artificial | aa | RISRRFS |
| | scFc VL CDR2 | | | |
| 47. | EGFRvIII × CD3- | artificial | aa | MQSTHVPRT |
| | scFc VL CDR3 | | | |
| 48. | EGFRvIII_CC × CD | artificial | aa | QVQLVESGGGVVQSGRSLRLSCAASGFTFRNYGMHWVR |
| | 3-scFc VH | | | QAPGKCLEWVAVIWYDGSDKYYADSVRGRFTISRDNSK |
| | | | | NTLYLQMNSLRAEDTAVYYCARDGYDILTGNPRDFDYW |
| | | | | GQGTLVTVSS |
| 49. | EGFRvIII_CC × CD | artificial | aa | DTVMTQTPLSSHVTLGQPASISCRSSQSLVHSDGNTYL |
| | 3-scFc VL | | | SWLQQRPGQPPRLLIYRISRRFSGVPDRFSGSGAGTDF |
| | | | | TLEISRVEAEDVGVYYCMQSTHVPRTFGCGTKVEIK |
| 50. | EGFRvIII_CC × CD | artificial | aa | QVQLVESGGGVVQSGRSLRLSCAASGFTFRNYGMHWVR |
| | 3-scFc scFv | | | QAPGKCLEWVAVIWYDGSDKYYADSVRGRFTISRDNSK |
| | | | | NTLYLQMNSLRAEDTAVYYCARDGYDILTGNPRDFDYW |
| | | | | GQGTLVTVSSGGGGSGGGGSGGGGSDTVMTQTPLSSHV |
| | | | | TLGQPASISCRSSQSLVHSDGNTYLSWLQQRPGQPPRL |
| | | | | LIYRISRRFSGVPDRFSGSGAGTDFTLEISRVEAEDVG |
| | | | | VVYYCMQSTHVPRTFGCGTKVEIK |
| 51. | EGFRvIII_CC × CD | artificial | aa | QVQLVESGGGVVQSGRSLRLSCAASGFTFRNYGMHWVR |
| | 3-scFc Bispecific | | | QAPGKCLEWVAVIWYDGSDKYYADSVRGRFTISRDNSK |
| | molecule | | | NTLYLQMNSLRAEDTAVYYCARDGYDILTGNPRDFDYW |
| | | | | GQGTLVTVSSGGGGSGGGGSGGGGSDTVMTQTPLSSHV |
| | | | | TLGQPASISCRSSQSLVHSDGNTYLSWLQQRPGQPPRL |
| | | | | LIYRISRRFSGVPDRFSGSGAGTDFTLEISRVEAEDVG |
| | | | | VYYCMQSTHVPRTFGCGTKVEIKSGGGGSEVQLVESGG |
| | | | | GLVQPGGSLKLSCAASGFTFNKYAMNWVRQAPGKGLEW |
| | | | | VARIRSKYNNYATYYADSVKDRFTISRDDSKNTAYLQM |
| | | | | NNLKTEDTAVYYCVRHGNFGNSYISYWAYWGQGTLVTV |
| | | | | SSGGGGSGGGGSGGGGSQTVVTQEPSLTVSPGGTVTLT |
| | | | | CGSSTGAVTSGNYPNWVQQKPGQAPRGLIGGTKFLAPG |
| | | | | TPARFSGSLLGGKAALTLSGVQPEDEAEYYCVLWYSNR |
| | | | | WVFGGGTKLTVL |
| 52. | EGFRvIII_CC × CD | artificial | aa | QVQLVESGGGVVQSGRSLRLSCAASGFTFRNYGMHWVR |
| | 3-scFc Bispecific | | | QAPGKCLEWVAVIWYDGSDKYYADSVRGRFTISRDNSK |
| | HLE molecule | | | NTLYLQMNSLRAEDTAVYYCARDGYDILTGNPRDFDYW |
| | | | | GQGTLVTVSSGGGGSGGGGSGGGGSDTVMTQTPLSSHV |
| | | | | TLGQPASISCRSSQSLVHSDGNTYLSWLQQRPGQPPRL |
| | | | | LIYRISRRFSGVPDRFSGSGAGTDFTLEISRVEAEDVG |
| | | | | VYYCMQSTHVPRTFGCGTKVEIKSGGGGSEVQLVESGG |
| | | | | GLVQPGGSLKLSCAASGFTFNKYAMNWVRQAPGKGLEW |
| | | | | VARIRSKYNNYATYYADSVKDRFTISRDDSKNTAYLQM |
| | | | | NNLKTEDTAVYYCVRHGNFGNSYISYWAYWGQGTLVTV |
| | | | | SSGGGGSGGGGSGGGGSQTVVTQEPSLTVSPGGTVTLT |
| | | | | CGSSTGAVTSGNYPNWVQQKPGQAPRGLIGGTKFLAPG |
| | | | | TPARFSGSLLGGKAALTLSGVQPEDEAEYYCVLWYSNR |
| | | | | WVFGGGTKLTVLGGGGDKTHTCPPCPAPELLGGPSVFL |
| | | | | FPPKPKDTLMISRTPEVTCVVVDVSHEDPEVKFNWYVD |
| | | | | GVEVHNAKTKPCEEQYGSTYRCVSVLTVLHQDWLNGKE |
| | | | | YKCKVSNKALPAPIEKTISKAKGQPREPQVYTLPPSRE |
| | | | | EMTKNQVSLTCLVKGFYPSDIAVEWESNGQPENNYKTT |
| | | | | PPVLDSDGSFFLYSKLTVDKSRWQQGNVFSCSVMHEAL |
| | | | | HNHYTQKSLSLSPGKGGGGSGGGGSGGGGSGGGGSGGG |
| | | | | GSGGGGSDKTHTCPPCPAPELLGGPSVFLFPPKPKDTL |
| | | | | MISRTPEVTCVVVDVSHEDPEVKFNWYVDGVEVHNAKT |
| | | | | KPCEEQYGSTYRCVSVLTVLHQDWLNGKEYKCKVSNKA |
| | | | | LPAPIEKTISKAKGQPREPQVYTLPPSREEMTKNQVSL |
| | | | | TCLVKGFYPSDIAVEWESNGQPENNYKTTPPVLDSDGS |
| | | | | FFLYSKLTVDKSRWQQGNVFSCSVMHEALHNHYTQKSL |
| | | | | SLSPGK |
| 53. | MSLN_5 VH | artificial | aa | DYYMT |
| | CDR1 | | | |
| 54. | MSLN_5 VH | artificial | aa | YISSSGSTIYYADSVKG |
| | CDR2 | | | |
| 55. | MSLN_5 VH | artificial | aa | DRNSHFDY |
| | CDR3 | | | |
| 56. | MSLN_5 VL | artificial | aa | RASQGINTWLA |
| | CDR1 | | | |
| 57. | MSLN_5 VL | artificial | aa | GASGLQS |
| | CDR2 | | | |
| 58. | MSLN_5 VL | artificial | aa | QQAKSFPRT |
| | CDR3 | | | |
| 59. | MSLN_5 VH | artificial | aa | QVQLVESGGGLVKPGGSLRLSCAASGFTFSDYYMTWIR |
| | | | | QAPGKGLEWLSYISSSGSTIYYADSVKGRFTISRDNAK |
| | | | | NSLFLQMNSLRAEDTAVYYCARDRNSHFDYWGQGTLVT |
| | | | | VSS |
| 60. | MSLN_5 VL | artificial | aa | DIQMTQSPSSVSASVGDRVTITCRASQGINTWLAWYQQ |
| | | | | KPGKAPKLLIYGASGLQSGVPSRFSGSGSGTDFTLTIS |
| | | | | SLQPEDFATYYCQQAKSFPRTFGQGTKVEIK |
| 61. | MSLN_5 scFv | artificial | aa | QVQLVESGGGLVKPGGSLRLSCAASGFTFSDYYMTWIR |
| | | | | QAPGKGLEWLSYISSSGSTIYYADSVKGRFTISRDNAK |
| | | | | NSLFLQMNSLRAEDTAVYYCARDRNSHFDYWGQGTLVT |
| | | | | VSSGGGGSGGGGSGGGGSDIQMTQSPSSVSASVGDRVT |
| | | | | ITCRASQGINTWLAWYQQKPGKAPKLLIYGASGLQSGV |
| | | | | PSRFSGSGSGTDFTLTISSLQPEDFATYYCQQAKSFPR |
| | | | | TFGQGTKVEIK |
| 62. | MSLN_5 × 12C0 | artificial | aa | QVQLVESGGGLVKPGGSLRLSCAASGFTFSDYYMTWIR |
| | bispecific | | | QAPGKGLEWLSYISSSGSTIYYADSVKGRFTISRDNAK |
| | molecule | | | NSLFLQMNSLRAEDTAVYYCARDRNSHFDYWGQGTLVT |
| | | | | VSSGGGGSGGGGSGGGGSDIQMTQSPSSVSASVGDRVT |
| | | | | ITCRASQGINTWLAWYQQKPGKAPKLLIYGASGLQSGV |
| | | | | PSRFSGSGSGTDFTLTISSLQPEDFATYYCQQAKSFPR |
| | | | | TFGQGTKVEIKSGGGGSEVQLVESGGGLVQPGGSLKLS |
| | | | | CAASGFTFNKYAMNWVRQAPGKGLEWVARIRSKYNNYA |
| | | | | TYYADSVKDRFTISRDDSKNTAYLQMNNLKTEDTAVYY |
| | | | | CVRHGNFGNSYISYWAYWGQGTLVTVSSGGGGSGGGGS |
| | | | | GGGGSQTVVTQEPSLTVSPGGTVTLTCGSSTGAVTSGN |
| | | | | YPNWVQQKPGQAPRGLIGGTKFLAPGTPARFSGSLLGG |
| | | | | KAALTLSGVQPEDEAEYYCVLWYSNRWVFGGGTKLTVL |
| 63. | MSLN_5 × CD3- | artificial | aa | QVQLVESGGGLVKPGGSLRLSCAASGFTFSDYYMTWIR |
| | scFc Bispecific | | | QAPGKGLEWLSYISSSGSTIYYADSVKGRFTISRDNAK |
| | HLE molecule | | | NSLFLQMNSLRAEDTAVYYCARDRNSHFDYWGQGTLVT |
| | | | | VSSGGGGSGGGGSGGGGSDIQMTQSPSSVSASVGDRVT |
| | | | | ITCRASQGINTWLAWYQQKPGKAPKLLIYGASGLQSGV |
| | | | | PSRFSGSGSGTDFTLTISSLQPEDFATYYCQQAKSFPR |
| | | | | TFGQGTKVEIKSGGGGSEVQLVESGGGLVQPGGSLKLS |
| | | | | CAASGFTFNKYAMNWVRQAPGKGLEWVARIRSKYNNYA |
| | | | | TYYADSVKDRFTISRDDSKNTAYLQMNNLKTEDTAVYY |
| | | | | CVRHGNFGNSYISYWAYWGQGTLVTVSSGGGGSGGGGS |
| | | | | GGGGSQTVVTQEPSLTVSPGGTVTLTCGSSTGAVTSGN |
| | | | | YPNWVQQKPGQAPRGLIGGTKFLAPGTPARFSGSLLGG |
| | | | | KAALTLSGVQPEDEAEYYCVLWYSNRWVFGGGTKLTVL |
| | | | | GGGGDKTHTCPPCPAPELLGGPSVFLFPPKPKDTLMIS |
| | | | | RTPEVTCVVVDVSHEDPEVKFNWYVDGVEVHNAKTKPC |
| | | | | EEQYGSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFFL |
| | | | | YSKLTVDKSRWQQGNVFSCSVMHEALHNHYTQKSLSLS |
| | | | | PGKGGGGSGGGGSGGGGSGGGGSGGGGSGGGGSDKTHT |
| | | | | CPPCPAPELLGGPSVFLFPPKPKDTLMISRTPEVTCVV |
| | | | | VDVSHEDPEVKFNWYVDGVEVHNAKTKPCEEQYGSTYR |
| | | | | CVSVLTVLHQDWLNGKEYKCKVSNKALPAPIEKTISKA |
| | | | | KGQPREPQVYTLPPSREEMTKNQVSLTCLVKGFYPSDI |
| | | | | AVEWESNGQPENNYKTTPPVLDSDGSFFLYSKLTVDKS |
| | | | | RWQQGNVFSCSVMHEALHNHYTQKSLSLSPGK |
| 64. | MSLN_5_CC × CD | artificial | aa | QVQLVESGGGLVKPGGSLRLSCAASGFTFSDHYMSWIR |
| | 3-scFc Bispecific | | | QAPGKCLEWFSYISSSGGIIYYADSVKGRFTISRDNAK |
| | HLE molecule | | | NSLYLQMNSLRAEDTAVYYCARDVGSHFDYWGQGTLVT |
| | | | | VSSGGGGSGGGGSGGGGSDIQMTQSPSSVSASVGDRVT |
| | | | | ITCRASQDISRWLAWYQQKPGKAPKLLISAASRLQSGV |
| | | | | PSRFSGSGSGTDFTLTISSLQPEDFAIYYCQQAKSFPR |
| | | | | TFGCGTKVEIKSGGGGSEVQLVESGGGLVQPGGSLKLS |
| | | | | CAASGFTFNKYAMNWVRQAPGKGLEWVARIRSKYNNYA |
| | | | | TYYADSVKDRFTISRDDSKNTAYLQMNNLKTEDTAVYY |
| | | | | CVRHGNFGNSYISYWAYWGQGTLVTVSSGGGGSGGGGS |
| | | | | GGGGSQTVVTQEPSLTVSPGGTVTLTCGSSTGAVTSGN |
| | | | | YPNWVQQKPGQAPRGLIGGTKFLAPGTPARFSGSLLGG |
| | | | | KAALTLSGVQPEDEAEYYCVLWYSNRWVFGGGTKLTVL |
| | | | | GGGGDKTHTCPPCPAPELLGGPSVFLFPPKPKDTLMIS |
| | | | | RTPEVTCVVVDVSHEDPEVKFNWYVDGVEVHNAKTKPC |
| | | | | EEQYGSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFFL |
| | | | | YSKLTVDKSRWQQGNVFSCSVMHEALHNHYTQKSLSLS |
| | | | | PGKGGGGSGGGGSGGGGSGGGGSGGGGSGGGGSDKTHT |
| | | | | CPPCPAPELLGGPSVFLFPPKPKDTLMISRTPEVTCVV |
| | | | | VDVSHEDPEVKFNWYVDGVEVHNAKTKPCEEQYGSTYR |
| | | | | CVSVLTVLHQDWLNGKEYKCKVSNKALPAPIEKTISKA |
| | | | | KGQPREPQVYTLPPSREEMTKNQVSLTCLVKGFYPSDI |
| | | | | AVEWESNGQPENNYKTTPPVLDSDGSFFLYSKLTVDKS |
| | | | | RWQQGNVFSCSVMHEALHNHYTQKSLSLSPGK |
| 65. | CDR-H1 of CDH19 | artificial | aa | SYGMH |
| | 65254.007 | | | |
| 66. | CDR-H2 of CDH19 | artificial | aa | FIWYEGSNKYYAESVKD |
| | 65254.007 | | | |
| 67. | CDR-H3 of CDH19 | artificial | aa | RAGIIGTIGYYYGMDV |
| | 65254.007 | | | |
| 68. | CDR-L1 of CDH19 | artificial | aa | SGDRLGEKYTS |
| | 65254.007 | | | |
| 69. | CDR-L2 of CDH19 | artificial | aa | QDTKRPS |
| | 65254.007 | | | |
| 70. | CDR-L3 of CDH19 | artificial | aa | QAWESSTVV |
| | 65254.007 | | | |
| 71. | VH of CDH19 | artificial | aa | QVQLVESGGGVVQPGGSLRLSCAASGFTFSSYGMHWVR |
| | 65254.007 | | | QAPGKGLEWVAFIWYEGSNKYYAESVKDRFTISRDNSK |
| | | | | NTLYLQMNSLRAEDTAVYYCARRAGIIGTIGYYYGMDV |
| | | | | WGQGTTVTVSS |
| 72. | VL of CDH19 | artificial | aa | SYELTQPPSVSVSPGQTASITCSGDRLGEKYTSWYQQR |
| | 65254.007 | | | PGQSPLLVIYQDTKRPSGIPERFSGSNSGNTATLTISG |
| | | | | TQAMDEADYYCQAWESSTVVFGGGTKLTVLS |
| 73. | VH-VL of CDH19 | artificial | aa | QVQLVESGGGVVQPGGSLRLSCAASGFTFSSYGMHWVR |
| | 65254.007 | | | QAPGKGLEWVAFIWYEGSNKYYAESVKDRFTISRDNSK |
| | | | | NTLYLQMNSLRAEDTAVYYCARRAGIIGTIGYYYGMDV |
| | | | | WGQGTTVTVSSGGGGSGGGGSGGGGSSYELTQPPSVSV |
| | | | | SPGQTASITCSGDRLGEKYTSWYQQRPGQSPLLVIYQD |
| | | | | TKRPSGIPERFSGSNSGNTATLTISGTQAMDEADYYCQ |
| | | | | AWESSTVVFGGGTKLTVLS |
| 74. | CDH19 65254.007 × | artificial | aa | QVQLVESGGGVVQPGGSLRLSCAASGFTFSSYGMHWVR |
| | I2C | | | QAPGKGLEWVAFIWYEGSNKYYAESVKDRFTISRDNSK |
| | | | | NTLYLQMNSLRAEDTAVYYCARRAGIIGTIGYYYGMDV |
| | | | | WGQGTTVTVSSGGGGSGGGGSGGGGSSYELTQPPSVSV |
| | | | | SPGQTASITCSGDRLGEKYTSWYQQRPGQSPLLVIYQD |
| | | | | TKRPSGIPERFSGSNSGNTATLTISGTQAMDEADYYCQ |
| | | | | AWESSTVVFGGGTKLTVLSGGGGSEVQLVESGGGLVQP |
| | | | | GGSLKLSCAASGFTENKYAMNWVRQAPGKGLEWVARIR |
| | | | | SKYNNYATYYADSVKDRFTISRDDSKNTAYLQMNNLKT |
| | | | | EDTAVYYCVRHGNFGNSYISYWAYWGQGTLVTVSSGGG |
| | | | | GSGGGGSGGGGSQTVVTQEPSLTVSPGGTVTLTCGSST |
| | | | | GAVTSGNYPNWVQQKPGQAPRGLIGGTKFLAPGTPARF |
| | | | | SGSLLGGKAALTLSGVQPEDEAEYYCVLWYSNRWVFGG |
| | | | | GTKLTVLHHHHHH |
| 75. | CDH19 65254.007 × | artificial | aa | QVQLVESGGGVVQPGGSLRLSCAASGFTFSSYGMHWVR |
| | I2C-scFc | | | QAPGKGLEWVAFIWYEGSNKYYAESVKDRFTISRDNSK |
| | Bispecific HLE | | | NTLYLQMNSLRAEDTAVYYCARRAGIIGTIGYYYGMDV |
| | molecule | | | WGQGTTVTVSSGGGGSGGGGSGGGGSSYELTQPPSVSV |
| | | | | SPGQTASITCSGDRLGEKYTSWYQQRPGQSPLLVIYQD |
| | | | | TKRPSGIPERFSGSNSGNTATLTISGTQAMDEADYYCQ |
| | | | | AWESSTVVFGGGTKLTVLSGGGGSEVQLVESGGGLVQP |
| | | | | GGSLKLSCAASGFTENKYAMNWVRQAPGKGLEWVARIR |
| | | | | SKYNNYATYYADSVKDRFTISRDDSKNTAYLQMNNLKT |
| | | | | EDTAVYYCVRHGNFGNSYISYWAYWGQGTLVTVSSGGG |
| | | | | GSGGGGSGGGGSQTVVTQEPSLTVSPGGTVTLTCGSST |
| | | | | GAVTSGNYPNWVQQKPGQAPRGLIGGTKFLAPGTPARF |
| | | | | SGSLLGGKAALTLSGVQPEDEAEYYCVLWYSNRWVFGG |
| | | | | GTKLTVLGGGGDKTHTCPPCPAPELLGGPSVFLFPPKP |
| | | | | KDTLMISRTPEVTCVVVDVSHEDPEVKFNWYVDGVEVH |
| | | | | NAKTKPCEEQYGSTYRCVSVLTVLHQDWLNGKEYKCKV |
| | | | | SNKALPAPIEKTISKAKGQPREPQVYTLPPSREEMTKN |
| | | | | QVSLTCLVKGFYPSDIAVEWESNGQPENNYKTTPPVLD |
| | | | | SDGSFFLYSKLTVDKSRWQQGNVFSCSVMHEALHNHYT |
| | | | | QKSLSLSPGKGGGGSGGGGSGGGGSGGGGSGGGGSGGG |
| | | | | GSDKTHTCPPCPAPELLGGPSVFLEPPKPKDTLMISRT |
| | | | | PEVTCVVVDVSHEDPEVKFNWYVDGVEVHNAKTKPCEE |
| | | | | QYGSTYRCVSVLTVLHQDWLNGKEYKCKVSNKALPAPI |
| | | | | EKTISKAKGQPREPQVYTLPPSREEMTKNQVSLTCLVK |
| | | | | GFYPSDIAVEWESNGQPENNYKTTPPVLDSDGSFFLYS |
| | | | | KLTVDKSRWQQGNVFSCSVMHEALHNHYTQKSLSLSPG |
| | | | | K |
| 76. | CDH19 65254.007 × | artificial | aa | QVQLVESGGGVVQPGGSLRLSCAASGFTFSSYGMHWVR |
| | I2C-scFc_delGK | | | QAPGKGLEWVAFIWYEGSNKYYAESVKDRFTISRDNSK |
| | Bispecific HLE | | | NTLYLQMNSLRAEDTAVYYCARRAGIIGTIGYYYGMDV |
| | molecule | | | WGQGTTVTVSSGGGGSGGGGSGGGGSSYELTQPPSVSV |
| | | | | SPGQTASITCSGDRLGEKYTSWYQQRPGQSPLLVIYQD |
| | | | | TKRPSGIPERFSGSNSGNTATLTISGTQAMDEADYYCQ |
| | | | | AWESSTVVFGGGTKLTVLSGGGGSEVQLVESGGGLVQP |
| | | | | GGSLKLSCAASGFTENKYAMNWVRQAPGKGLEWVARIR |
| | | | | SKYNNYATYYADSVKDRFTISRDDSKNTAYLQMNNLKT |
| | | | | EDTAVYYCVRHGNFGNSYISYWAYWGQGTLVTVSSGGG |
| | | | | GSGGGGSGGGGSQTVVTQEPSLTVSPGGTVTLTCGSST |
| | | | | GAVTSGNYPNWVQQKPGQAPRGLIGGTKFLAPGTPARF |
| | | | | SGSLLGGKAALTLSGVQPEDEAEYYCVLWYSNRWVFGG |
| | | | | GTKLTVLGGGGDKTHTCPPCPAPELLGGPSVFLFPPKP |
| | | | | KDTLMISRTPEVTCVVVDVSHEDPEVKFNWYVDGVEVH |
| | | | | NAKTKPCEEQYGSTYRCVSVLTVLHQDWLNGKEYKCKV |
| | | | | SNKALPAPIEKTISKAKGQPREPQVYTLPPSREEMTKN |
| | | | | QVSLTCLVKGFYPSDIAVEWESNGQPENNYKTTPPVLD |
| | | | | SDGSFFLYSKLTVDKSRWQQGNVFSCSVMHEALHNHYT |
| | | | | QKSLSLSPGGGGSGGGGSGGGGSGGGGSGGGGSGGGGS |
| | | | | DKTHTCPPCPAPELLGGPSVFLEPPKPKDTLMISRTPE |
| | | | | VTCVVVDVSHEDPEVKFNWYVDGVEVHNAKTKPCEEQY |
| | | | | GSTYRCVSVLTVLHQDWLNGKEYKCKVSNKALPAPIEK |
| | | | | TISKAKGQPREPQVYTLPPSREEMTKNQVSLTCLVKGF |
| | | | | YPSDIAVEWESNGQPENNYKTTPPVLDSDGSFFLYSKL |
| | | | | TVDKSRWQQGNVFSCSVMHEALHNHYTQKSLSLSPGK |
| 77. | CDH19 | artificial | aa | QVQLVESGGGVVQPGGSLRLSCAASGFTFSSYGMHWVR |
| | 65254.007_CC × | | | QAPGKCLEWVAFIWYEGSNKYYAESVKDRFTISRDNSK |
| | I2C-scFc VH | | | NTLYLQMNSLRAEDTAVYYCARRAGIIGTIGYYYGMDV |
| | | | | WGQGTTVTVSS |
| 78. | CDH19 | artificial | aa | SYELTQPPSVSVSPGQTASITCSGDRLGEKYTSWYQQR |
| | 65254.007_CC × | | | PGQSPLLVIYQDTKRPSGIPERFSGSNSGNTATLTISG |
| | I2C-scFc VL | | | TQAMDEADYYCQAWESSTVVFGCGTKLTVL |
| 79. | CDH19 | artificial | aa | QVQLVESGGGVVQPGGSLRLSCAASGFTFSSYGMHWVR |
| | 65254.007_CC × | | | QAPGKCLEWVAFIWYEGSNKYYAESVKDRFTISRDNSK |
| | I2C-scFc scFv | | | NTLYLQMNSLRAEDTAVYYCARRAGIIGTIGYYYGMDV |
| | | | | WGQGTTVTVSSGGGGSGGGGSGGGGSSYELTQPPSVSV |
| | | | | SPGQTASITCSGDRLGEKYTSWYQQRPGQSPLLVIYQD |
| | | | | TKRPSGIPERFSGSNSGNTATLTISGTQAMDEADYYCQ |
| | | | | AWESSTVVFGCGTKLTVL |
| 80. | CDH19 | artificial | aa | QVQLVESGGGVVQPGGSLRLSCAASGFTFSSYGMHWVR |
| | 65254.007_CC × | | | QAPGKCLEWVAFIWYEGSNKYYAESVKDRFTISRDNSK |
| | I2C-scFc | | | NTLYLQMNSLRAEDTAVYYCARRAGIIGTIGYYYGMDV |
| | Bispecific | | | WGQGTTVTVSSGGGGSGGGGSGGGGSSYELTQPPSVSV |
| | molecule | | | SPGQTASITCSGDRLGEKYTSWYQQRPGQSPLLVIYQD |
| | | | | TKRPSGIPERFSGSNSGNTATLTISGTQAMDEADYYCQ |
| | | | | AWESSTVVFGCGTKLTVLSGGGGSEVQLVESGGGLVQP |
| | | | | GGSLKLSCAASGFTENKYAMNWVRQAPGKGLEWVARIR |
| | | | | SKYNNYATYYADSVKDRFTISRDDSKNTAYLQMNNLKT |
| | | | | EDTAVYYCVRHGNFGNSYISYWAYWGQGTLVTVSSGGG |
| | | | | GSGGGGSGGGGSQTVVTQEPSLTVSPGGTVTLTCGSST |
| | | | | GAVTSGNYPNWVQQKPGQAPRGLIGGTKFLAPGTPARF |
| | | | | SGSLLGGKAALTLSGVQPEDEAEYYCVLWYSNRWVFGG |
| | | | | GTKLTVL |
| 81. | CDH19 | artificial | aa | QVQLVESGGGVVQPGGSLRLSCAASGFTFSSYGMHWVR |
| | 65254.007_CC × | | | QAPGKCLEWVAFIWYEGSNKYYAESVKDRFTISRDNSK |
| | I2C-scFc | | | NTLYLQMNSLRAEDTAVYYCARRAGIIGTIGYYYGMDV |
| | Bispecific HLE | | | WGQGTTVTVSSGGGGSGGGGSGGGGSSYELTQPPSVSV |
| | molecule | | | SPGQTASITCSGDRLGEKYTSWYQQRPGQSPLLVIYQD |
| | | | | TKRPSGIPERFSGSNSGNTATLTISGTQAMDEADYYCQ |
| | | | | AWESSTVVFGCGTKLTVLSGGGGSEVQLVESGGGLVQP |
| | | | | GGSLKLSCAASGFTENKYAMNWVRQAPGKGLEWVARIR |
| | | | | SKYNNYATYYADSVKDRFTISRDDSKNTAYLQMNNLKT |
| | | | | EDTAVYYCVRHGNFGNSYISYWAYWGQGTLVTVSSGGG |
| | | | | GSGGGGSGGGGSQTVVTQEPSLTVSPGGTVTLTCGSST |
| | | | | GAVTSGNYPNWVQQKPGQAPRGLIGGTKFLAPGTPARF |
| | | | | SGSLLGGKAALTLSGVQPEDEAEYYCVLWYSNRWVFGG |
| | | | | GTKLTVLGGGGDKTHTCPPCPAPELLGGPSVFLFPPKP |
| | | | | KDTLMISRTPEVTCVVVDVSHEDPEVKFNWYVDGVEVH |
| | | | | NAKTKPCEEQYGSTYRCVSVLTVLHQDWLNGKEYKCKV |
| | | | | SNKALPAPIEKTISKAKGQPREPQVYTLPPSREEMTKN |
| | | | | QVSLTCLVKGFYPSDIAVEWESNGQPENNYKTTPPVLD |
| | | | | SDGSFFLYSKLTVDKSRWQQGNVFSCSVMHEALHNHYT |
| | | | | QKSLSLSPGKGGGGSGGGGSGGGGSGGGGSGGGGSGGG |
| | | | | GSDKTHTCPPCPAPELLGGPSVFLEPPKPKDTLMISRT |
| | | | | PEVTCVVVDVSHEDPEVKFNWYVDGVEVHNAKTKPCEE |
| | | | | QYGSTYRCVSVLTVLHQDWLNGKEYKCKVSNKALPAPI |
| | | | | EKTISKAKGQPREPQVYTLPPSREEMTKNQVSLTCLVK |
| | | | | GFYPSDIAVEWESNGQPENNYKTTPPVLDSDGSFELYS |
| | | | | KLTVDKSRWQQGNVFSCSVMHEALHNHYTQKSLSLSPG |
| | | | | K |
| 82. | CDH19 | artificial | aa | QVQLVESGGGVVQPGGSLRLSCAASGFTFSSYGMHWVR |
| | 65254.007_CC × | | | QAPGKCLEWVAFIWYEGSNKYYAESVKDRFTISRDNSK |
| | I2C-scFc_delGK | | | NTLYLQMNSLRAEDTAVYYCARRAGIIGTIGYYYGMDV |
| | Bispecific HLE | | | WGQGTTVTVSSGGGGSGGGGSGGGGSSYELTQPPSVSV |
| | molecule | | | SPGQTASITCSGDRLGEKYTSWYQQRPGQSPLLVIYQD |
| | | | | TKRPSGIPERFSGSNSGNTATLTISGTQAMDEADYYCQ |
| | | | | AWESSTVVFGCGTKLTVLSGGGGSEVQLVESGGGLVQP |
| | | | | GGSLKLSCAASGFTENKYAMNWVRQAPGKGLEWVARIR |
| | | | | SKYNNYATYYADSVKDRFTISRDDSKNTAYLQMNNLKT |
| | | | | EDTAVYYCVRHGNFGNSYISYWAYWGQGTLVTVSSGGG |
| | | | | GSGGGGSGGGGSQTVVTQEPSLTVSPGGTVTLTCGSST |
| | | | | GAVTSGNYPNWVQQKPGQAPRGLIGGTKFLAPGTPARF |
| | | | | SGSLLGGKAALTLSGVQPEDEAEYYCVLWYSNRWVFGG |
| | | | | GTKLTVLGGGGDKTHTCPPCPAPELLGGPSVFLFPPKP |
| | | | | KDTLMISRTPEVTCVVVDVSHEDPEVKFNWYVDGVEVH |
| | | | | NAKTKPCEEQYGSTYRCVSVLTVLHQDWLNGKEYKCKV |
| | | | | SNKALPAPIEKTISKAKGQPREPQVYTLPPSREEMTKN |
| | | | | QVSLTCLVKGFYPSDIAVEWESNGQPENNYKTTPPVLD |
| | | | | SDGSFFLYSKLTVDKSRWQQGNVFSCSVMHEALHNHYT |
| | | | | QKSLSLSPGGGGSGGGGSGGGGSGGGGSGGGGSGGGGS |
| | | | | DKTHTCPPCPAPELLGGPSVFLEPPKPKDTLMISRTPE |
| | | | | VTCVVVDVSHEDPEVKFNWYVDGVEVHNAKTKPCEEQY |
| | | | | GSTYRCVSVLTVLHQDWLNGKEYKCKVSNKALPAPIEK |
| | | | | TISKAKGQPREPQVYTLPPSREEMTKNQVSLTCLVKGF |
| | | | | YPSDIAVEWESNGQPENNYKTTPPVLDSDGSFELYSKL |
| | | | | TVDKSRWQQGNVFSCSVMHEALHNHYTQKSLSLSPGK |
| 83. | FLT3_7 | artificial | aa | NARMGVS |
| | A8 × CD3-scFc | | | |
| | VH CDR1 | | | |
| 84. | FLT3_7 | artificial | aa | HIFSNDEKSYSTSLKN |
| | A8 × CD3-scFc | | | |
| | VH CDR2 | | | |
| 85. | FLT3_7 | artificial | aa | IVGYGSGWYGFFDY |
| | A8 × CD3-scFc | | | |
| | VH CDR3 | | | |
| 86. | FLT3_7 | artificial | aa | RASQGIRNDLG |
| | A8 × CD3-scFc | | | |
| | VL CDR1 | | | |
| 87. | FLT3_7 | artificial | aa | AASTLQS |
| | A8 × CD3-scFc | | | |
| | VL CDR2 | | | |
| 88. | FLT3_7 | artificial | aa | LQHNSYPLT |
| | A8 × CD3-scFc | | | |
| | VL CDR3 | | | |
| 89. | FLT3_7 | artificial | aa | QVTLKESGPTLVKPTETLTLTCTLSGFSLNNARMGV |
| | A8 × CD3-scFc | | | SWIRQPPGKCLEWLAHIFSNDEKSYSTSLKNRLTIS |
| | VH | | | KDSSKTQVVLTMTNVDPVDTATYYCARIVGYGSGWY |
| | | | | GFFDYWGQGTLVTVSS |
| 90. | FLT3_A8-scFc | artificial | aa | DIQMTQSPSSLSASVGDRVTITCRASQGIRNDLGWY |
| | VL | | | QQKPGKAPKRLIYAASTLQSGVPSRFSGSGSGTEFT |
| | | | | LTISSLQPEDFATYYCLQHNSYPLTFGCGTKVEIK |
| 91. | FLT3_7 | artificial | aa | QVTLKESGPTLVKPTETLTLTCTLSGFSLNNARMGV |
| | A8 × CD3-scFv | | | SWIRQPPGKCLEWLAHIFSNDEKSYSTSLKNRLTIS |
| | | | | KDSSKTQVVLTMTNVDPVDTATYYCARIVGYGSGWY |
| | | | | GFFDYWGQGTLVTVSSGGGGSGGGGSGGGGSDIQMT |
| | | | | QSPSSLSASVGDRVTITCRASQGIRNDLGWYQQKPG |
| | | | | KAPKRLIYAASTLQSGVPSRFSGSGSGTEFTLTISS |
| | | | | LQPEDFATYYCLQHNSYPLTFGCGTKVEIK |
| 92. | FLT3_7 | artificial | aa | QVTLKESGPTLVKPTETLTLTCTLSGFSLNNARMGV |
| | A8 × CD3 | | | SWIRQPPGKCLEWLAHIFSNDEKSYSTSLKNRLTIS |
| | Bispecific | | | KDSSKTQVVLTMTNVDPVDTATYYCARIVGYGSGWY |
| | molecule | | | GFFDYWGQGTLVTVSSGGGGSGGGGSGGGGSDIQMT |
| | | | | QSPSSLSASVGDRVTITCRASQGIRNDLGWYQQKPG |
| | | | | KAPKRLIYAASTLQSGVPSRFSGSGSGTEFTLTISS |
| | | | | LQPEDFATYYCLQHNSYPLTFGCGTKVEIKSGGGGS |
| | | | | EVQLVESGGGLVQPGGSLKLSCAASGFTFNKYAMNW |
| | | | | VRQAPGKGLEWVARIRSKYNNYATYYADSVKDRFTI |
| | | | | SRDDSKNTAYLQMNNLKTEDTAVYYCVRHGNFGNSY |
| | | | | ISYWAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALT |
| | | | | LSGVQPEDEAEYYCVLWYSNRWVFGGGTKLTVL |
| 93. | FLT3_7 | artificial | aa | QVTLKESGPTLVKPTETLTLTCTLSGFSLNNARMGV |
| | A8 × CD3-scFc | | | SWIRQPPGKCLEWLAHIFSNDEKSYSTSLKNRLTIS |
| | Bispecific HLE | | | KDSSKTQVVLTMTNVDPVDTATYYCARIVGYGSGWY |
| | molecule | | | GFFDYWGQGTLVTVSSGGGGSGGGGSGGGGSDIQMT |
| | | | | QSPSSLSASVGDRVTITCRASQGIRNDLGWYQQKPG |
| | | | | KAPKRLIYAASTLQSGVPSRFSGSGSGTEFTLTISS |
| | | | | LQPEDFATYYCLQHNSYPLTFGCGTKVEIKSGGGGS |
| | | | | EVQLVESGGGLVQPGGSLKLSCAASGFTFNKYAMNW |
| | | | | VRQAPGKGLEWVARIRSKYNNYATYYADSVKDRFTI |
| | | | | SRDDSKNTAYLQMNNLKTEDTAVYYCVRHGNFGNSY |
| | | | | ISYWAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALT |
| | | | | LSGVQPEDEAEYYCVLWYSNRWVFGGGTKLTVLGGG |
| | | | | GDKTHTCPPCPAPELLGGPSVFLFPPKPKDTLMISR |
| | | | | TPEVTCVVVDVSHEDPEVKFNWYVDGVEVHNAKTKP |
| | | | | CEEQYGSTYRCVSVLTVLHQDWLNGKEYKCKVSNKA |
| | | | | LPAPIEKTISKAKGQPREPQVYTLPPSREEMTKNQV |
| | | | | SLTCLVKGFYPSDIAVEWESNGQPENNYKTTPPVLD |
| | | | | SDGSFFLYSKLTVDKSRWQQGNVFSCSVMHEALHNH |
| | | | | YTQKSLSLSPGKGGGGSGGGGSGGGGSGGGGSGGGG |
| | | | | SGGGGSDKTHTCPPCPAPELLGGPSVFLFPPKPKDT |
| | | | | LMISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEM |
| | | | | TKNQVSLTCLVKGFYPSDIAVEWESNGQPENNYKTT |
| | | | | PPVLDSDGSFFLYSKLTVDKSRWQQGNVFSCSVMHE |
| | | | | ALHNHYTQKSLSLSPGK |
| 94. | VH CDR1 | artificial | aa | SYYWS |
| | DLL3_1_CC_ | | | |
| | delGK | | | |
| 95. | VH CDR2 | artificial | aa | YVYYSGTTNYNPSLKS |
| | DLL3_1_CC_ | | | |
| | delGK | | | |
| 96. | VH CDR3 | artificial | aa | IAVTGFYFDY |
| | DLL3_1_CC_ | | | |
| | delGK | | | |
| 97. | VL CDR1 | artificial | aa | RASQRVNNNYLA |
| | DLL3_1_CC_ | | | |
| | delGK | | | |
| 98. | VL CDR2 | artificial | aa | GASSRAT |
| | DLL3_1_CC_ | | | |
| | delGK | | | |
| 99. | VL CDR3 | artificial | aa | QQYDRSPLT |
| | DLL3_1_CC_ | | | |
| | delGK | | | |
| 100. | VH | artificial | aa | QVQLQESGPGLVKPSETLSLTCTVSGGSISSYYWSW |
| | DLL3_1_CC_ | | | IRQPPGKCLEWIGYVYYSGTTNYNPSLKSRVTISVD |
| | delGK | | | TSKNQFSLKLSSVTAADTAVYYCASIAVTGFYFDYW |
| | | | | GQGTLVTVSS |
| 101. | VL | artificial | aa | EIVLTQSPGTLSLSPGERVTLSCRASQRVNNNYLAW |
| | DLL3_1_CC_ | | | YQQRPGQAPRLLIYGASSRATGIPDRFSGSGSGTDF |
| | delGK | | | TLTISRLEPEDFAVYYCQQYDRSPLTFGCGTKLEIK |
| 102. | DLL3_1_CC_ | artificial | aa | QVQLQESGPGLVKPSETLSLTCTVSGGSISSYYWSW |
| | delGK | | | IRQPPGKCLEWIGYVYYSGTTNYNPSLKSRVTISVD |
| | | | | TSKNQFSLKLSSVTAADTAVYYCASIAVTGFYFDYW |
| | | | | GQGTLVTVSSGGGGSGGGGSGGGGSEIVLTQSPGTL |
| | | | | SLSPGERVTLSCRASQRVNNNYLAWYQQRPGQAPRL |
| | | | | LIYGASSRATGIPDRFSGSGSGTDFTLTISRLEPED |
| | | | | FAVYYCQQYDRSPLTFGCGTKLEIK |
| 103. | DLL3_1_CC × | artificial | aa | QVQLQESGPGLVKPSETLSLTCTVSGGSISSYYWSW |
| | CD3_delGK | | | IRQPPGKCLEWIGYVYYSGTTNYNPSLKSRVTISVD |
| | Bispecific | | | TSKNQFSLKLSSVTAADTAVYYCASIAVTGFYFDYW |
| | molecule | | | GQGTLVTVSSGGGGSGGGGSGGGGSEIVLTQSPGTL |
| | | | | SLSPGERVTLSCRASQRVNNNYLAWYQQRPGQAPRL |
| | | | | LIYGASSRATGIPDRFSGSGSGTDFTLTISRLEPED |
| | | | | FAVYYCQQYDRSPLTFGCGTKLEIKSGGGGSEVQLV |
| | | | | ESGGGLVQPGGSLKLSCAASGFTFNKYAMNWVRQAP |
| | | | | GKGLEWVARIRSKYNNYATYYADSVKDRFTISRDDS |
| | | | | KNTAYLQMNNLKTEDTAVYYCVRHGNFGNSYISYWA |
| | | | | YWGQGTLVTVSSGGGGSGGGGSGGGGSQTVVTQEPS |
| | | | | LTVSPGGTVTLTCGSSTGAVTSGNYPNWVQQKPGQA |
| | | | | PRGLIGGTKFLAPGTPARFSGSLLGGKAALTLSGVQ |
| | | | | PEDEAEYYCVLWYSNRWVFGGGTKLTVL |
| 104. | DLL3_1_CC × | artificial | aa | QVQLQESGPGLVKPSETLSLTCTVSGGSISSYYWSW |
| | CD3- | | | IRQPPGKCLEWIGYVYYSGTTNYNPSLKSRVTISVD |
| | scFc_delGK | | | TSKNQFSLKLSSVTAADTAVYYCASIAVTGFYFDYW |
| | Bispecific HLE | | | GQGTLVTVSSGGGGSGGGGSGGGGSEIVLTQSPGTL |
| | molecule | | | SLSPGERVTLSCRASQRVNNNYLAWYQQRPGQAPRL |
| | | | | LIYGASSRATGIPDRFSGSGSGTDFTLTISRLEPED |
| | | | | FAVYYCQQYDRSPLTFGCGTKLEIKSGGGGSEVQLV |
| | | | | ESGGGLVQPGGSLKLSCAASGFTFNKYAMNWVRQAP |
| | | | | GKGLEWVARIRSKYNNYATYYADSVKDRFTISRDDS |
| | | | | KNTAYLQMNNLKTEDTAVYYCVRHGNFGNSYISYWA |
| | | | | YWGQGTLVTVSSGGGGSGGGGSGGGGSQTVVTQEPS |
| | | | | LTVSPGGTVTLTCGSSTGAVTSGNYPNWVQQKPGQA |
| | | | | PRGLIGGTKFLAPGTPARFSGSLLGGKAALTLSGVQ |
| | | | | PEDEAEYYCVLWYSNRWVFGGGTKLTVLGGGGDKTH |
| | | | | TCPPCPAPELLGGPSVFLFPPKPKDTLMISRTPEVT |
| | | | | CVVVDVSHEDPEVKFNWYVDGVEVHNAKTKPCEEQY |
| | | | | GSTYRCVSVLTVLHQDWLNGKEYKCKVSNKALPAPI |
| | | | | EKTISKAKGQPREPQVYTLPPSREEMTKNQVSLTCL |
| | | | | VKGFYPSDIAVEWESNGQPENNYKTTPPVLDSDGSF |
| | | | | FLYSKLTVDKSRWQQGNVFSCSVMHEALHNHYTQKS |
| | | | | LSLSPGGGGSGGGGSGGGGSGGGGSGGGGSGGGGSD |
| | | | | KTHTCPPCPAPELLGGPSVFLFPPKPKDTLMISRTP |
| | | | | EVTCVVVDVSHEDPEVKFNWYVDGVEVHNAKTKPCE |
| | | | | EQYGSTYRCVSVLTVLHQDWLNGKEYKCKVSNKALP |
| | | | | APIEKTISKAKGQPREPQVYTLPPSREEMTKNQVSL |
| | | | | TCLVKGFYPSDIAVEWESNGQPENNYKTTPPVLDSD |
| | | | | GSFFLYSKLTVDKSRWQQGNVFSCSVMHEALHNHYT |
| | | | | QKSLSLSPGK |
| 105. | VH CDR1 | artificial | aa | SYGMH |
| | CD19 97- | | | |
| | G1RE-C2 | | | |
| 106. | VH CDR2 | artificial | aa | VISYEGSNKYYAESVKG |
| | CD19 97- | | | |
| | G1RE-C2 | | | |
| 107. | VH CDR3 | artificial | aa | DRGTIFGNYGLEV |
| | CD19 97- | | | |
| | G1RE-C2 | | | |
| 108. | VH CD19 97- | artificial | aa | QVQLVESGGGVVQPGRSLRLSCAASGFTFSSYGMHW |
| | G1RE-C2 CC | | | VRQAPGKCLEWVAVISYEGSNKYYAESVKGRFTISR |
| | | | | DNSKNTLYLQMNSLRDEDTAVYYCARDRGTIFGNYG |
| | | | | LEVWGQGTTVTVSS |
| 109. | VL CDR1 CD19 | artificial | aa | RSSQSLLHKNAFNYLD |
| | 97-G1RE-C2 | | | |
| 110. | VL CDR2 CD19 | artificial | aa | LGSNRAS |
| | 97-G1RE-C2 | | | |
| 111. | VL CDR3 CD19 | artificial | aa | MQALQTPFT |
| | 97-G1RE-C2 | | | |
| 112. | VL CD19 97- | artificial | aa | DIVMTQSPLSLPVISGEPASISCRSSQSLLHKNAFN |
| | G1RE-C2 CC | | | YLDWYLQKPGQSPQLLIYLGSNRASGVPDRFSGSGS |
| | | | | GTDFTLKISRVEAEDVGVYYCMQALQTPFTFGCGTK |
| | | | | VD IK |
| 113. | CD19 97- | artificial | aa | MDMRVPAQLLGLLLLWLRGARCDIVMTQSPLSLPVI |
| | G1RE-C2 CC × | | | SGEPASISCRSSQSLLHKNAFNYLDWYLQKPGQSPQ |
| | I2C0 | | | LLIYLGSNRASGVPDRFSGSGSGTDFTLKISRVEAE |
| | | | | DVGVYYCMQALQTPFTFGCGTKVDIKGGGGSGGGGS |
| | | | | GGGGSQVQLVESGGGVVQPGRSLRLSCAASGFTFSS |
| | | | | YGMHWVRQAPGKCLEWVAVISYEGSNKYYAESVKGR |
| | | | | FTISRDNSKNTLYLQMNSLRDEDTAVYYCARDRGTI |
| | | | | FGNYGLEVWGQGTTVTVSSGGGGSEVQLVESGGGLV |
| | | | | QPGGSLKLSCAASGFTFNKYAMNWVRQAPGKGLEWV |
| | | | | ARIRSKYNNYATYYADSVKDRFTISRDDSKNTAYLQ |
| | | | | MNNLKTEDTAVYYCVRHGNFGNSYISYWAYWGQGTL |
| | | | | VTVSSGGGGSGGGGSGGGGSQTVVTQEPSLTVSPGG |
| | | | | TVTLTCGSSTGAVTSGNYPNWVQQKPGQAPRGLIGG |
| | | | | TKFLAPGTPARFSGSLLGGKAALTLSGVQPEDEAEY |
| | | | | YCVLWYSNRWVFGGGTKLTVL |
| 114. | CD19 97- | artificial | aa | MDMRVPAQLLGLLLLWLRGARCDIVMTQSPLSLPVI |
| | G1RE-C2 CC × | | | SGEPASISCRSSQSLLHKNAFNYLDWYLQKPGQSPQ |
| | I2C0-scFc | | | LLIYLGSNRASGVPDRFSGSGSGTDFTLKISRVEAE |
| | | | | DVGVYYCMQALQTPFTFGCGTKVDIKGGGGSGGGGS |
| | | | | GGGGSQVQLVESGGGVVQPGRSLRLSCAASGFTFSS |
| | | | | YGMHWVRQAPGKCLEWVAVISYEGSNKYYAESVKGR |
| | | | | FTISRDNSKNTLYLQMNSLRDEDTAVYYCARDRGTI |
| | | | | FGNYGLEVWGQGTTVTVSSGGGGSEVQLVESGGGLV |
| | | | | QPGGSLKLSCAASGFTFNKYAMNWVRQAPGKGLEWV |
| | | | | ARIRSKYNNYATYYADSVKDRFTISRDDSKNTAYLQ |
| | | | | MNNLKTEDTAVYYCVRHGNFGNSYISYWAYWGQGTL |
| | | | | VTVSSGGGGSGGGGSGGGGSQTVVTQEPSLTVSPGG |
| | | | | TVTLTCGSSTGAVTSGNYPNWVQQKPGQAPRGLIGG |
| | | | | TKFLAPGTPARFSGSLLGGKAALTLSGVQPEDEAEY |
| | | | | YCVLWYSNRWVFGGGTKLTVLGGGGDKTHTCPPCPA |
| | | | | PELLGGPSVFLFPPKPKDTLMISRTPEVTCVVVDVS |
| | | | | HEDPEVKFNWYVDGVEVHNAKTKPCEEQYGSTYRCV |
| | | | | SVLTVLHQDWLNGKEYKCKVSNKALPAPIEKTISKA |
| | | | | KGQPREPQVYTLPPSREEMTKNQVSLTCLVKGFYPS |
| | | | | DIAVEWESNGQPENNYKTTPPVLDSDGSFFLYSKLT |
| | | | | VDKSRWQQGNVFScSVMHEALHNHYTQKSLSLSPGK |
| | | | | GGGGSGGGGSGGGGSGGGGSGGGGSGGGGSDKTHTC |
| | | | | PPCPAPELLGGPSVFLFPPKPKDTLMISRTPEVTCV |
| | | | | VVDVSHEDPEVKFNWYVDGVEVHNAKTKPCEEQYGS |
| | | | | TYRCVSVLTVLHQDWLNGKEYKCKVSNKALPAPIEK |
| | | | | TISKAKGQPREPQVYTLPPSREEMTKNQVSLTCLVK |
| | | | | GFYPSDIAVEWESNGQPENNYKTTPPVLDSDGSFFL |
| | | | | YSKLTVDKSRWQQGNVFSCSVMHEALHNHYTQKSLS |
| 115. | VH CDR1 | artificial | aa | SYPIN |
| | CDH3 G8A 6- | | | |
| | B12 | | | |
| 116. | VH CDR2 | artificial | aa | VIWTGGGTNYASSVKG |
| | CDH3 G8A 6- | | | |
| | B12 | | | |
| 117. | VH CDR3 | artificial | aa | SRGVYDFDGRGAMDY |
| | CDH3 G8A 6- | | | |
| | B12 | | | |
| 118. | VL CDR1 | artificial | aa | KSSQSLLYSSNQKNYFA |
| | CDH3 G8A 6- | | | |
| | B12 | | | |
| 119. | VL CDR2 | artificial | aa | WASTRES |
| | CDH3 G8A 6- | | | |
| | B12 | | | |
| 120. | VL CDR3 | artificial | aa | QQYYSYPYT |
| | CDH3 G8A 6- | | | |
| | B12 | | | |
| 121. | VH CDH3 G8A | artificial | aa | EVQLLESGGGLVQPGGSLRLSCAASGFSFSSYPINW |
| | 6-B12 | | | VRQAPGKGLEWVGVIWTGGGTNYASSVKGRFTISRD |
| | | | | NSKNTVYLQMNSLRAEDTAVYYCAKSRGVYDFDGRG |
| | | | | AMDYWGQGTLVTVSS |
| 122. | VL CDH3 G8A | artificial | aa | DIVMTQSPDSLAVSLGERATINCKSSQSLLYSSNQK |
| | 6-B12 | | | NYFAWYQQKPGQPPKLLIYWASTRESGVPDRFSGSG |
| | | | | SGTDFTLTISSLQAEDVAVYYCQQYYSYPYTFGQGT |
| | | | | KLEIK |
| 123. | CDH3 G8A 6- | artificial | aa | EVQLLESGGGLVQPGGSLRLSCAASGFSFSSYPINW |
| | B12 scFv | | | VRQAPGKGLEWVGVIWTGGGTNYASSVKGRFTISRD |
| | | | | NSKNTVYLQMNSLRAEDTAVYYCAKSRGVYDFDGRG |
| | | | | AMDYWGQGTLVTVSSGGGGSGGGGSGGGGSDIVMTQ |
| | | | | SPDSLAVSLGERATINCKSSQSLLYSSNQKNYFAWY |
| | | | | QQKPGQPPKLLIYWASTRESGVPDRFSGSGSGTDFT |
| | | | | LTISSLQAEDVAVYYCQQYYSYPYTFGQGTKLEIK |
| 124. | CDH3 G8A 6- | artificial | aa | EVQLLESGGGLVQPGGSLRLSCAASGFSFSSYPINW |
| | B12 × 12C0 | | | VRQAPGKGLEWVGVIWTGGGTNYASSVKGRFTISRD |
| | bispecific | | | NSKNTVYLQMNSLRAEDTAVYYCAKSRGVYDFDGRG |
| | molecule | | | AMDYWGQGTLVTVSSGGGGSGGGGSGGGGSDIVMTQ |
| | | | | SPDSLAVSLGERATINCKSSQSLLYSSNQKNYFAWY |
| | | | | QQKPGQPPKLLIYWASTRESGVPDRFSGSGSGTDFT |
| | | | | LTISSLQAEDVAVYYCQQYYSYPYTFGQGTKLEIKS |
| | | | | GGGGSEVQLVESGGGLVQPGGSLKLSCAASGFTFNK |
| | | | | YAMNWVRQAPGKGLEWVARIRSKYNNYATYYADSVK |
| | | | | DRFTISRDDSKNTAYLQMNNLKTEDTAVYYCVRHGN |
| | | | | FGNSYISYWAYWGQGTLVTVSSGGGGSGGGGSGGGG |
| | | | | SQTVVTQEPSLTVSPGGTVTLTCGSSTGAVTSGNYP |
| | | | | NWVQQKPGQAPRGLIGGTKFLAPGTPARFSGSLLGG |
| | | | | KAALTLSGVQPEDEAEYYCVLWYSNRWVFGGGTKLT |
| | | | | VL |
| 125. | CDH3 G8A 6- | artificial | aa | EVQLLESGGGLVQPGGSLRLSCAASGFSFSSYPINW |
| | B12 × I2C0 | | | VRQAPGKGLEWVGVIWTGGGTNYASSVKGRFTISRD |
| | bispecific | | | NSKNTVYLQMNSLRAEDTAVYYCAKSRGVYDFDGRG |
| | molecule HLE | | | AMDYWGQGTLVTVSSGGGGSGGGGSGGGGSDIVMTQ |
| | | | | SPDSLAVSLGERATINCKSSQSLLYSSNQKNYFAWY |
| | | | | QQKPGQPPKLLIYWASTRESGVPDRFSGSGSGTDFT |
| | | | | LTISSLQAEDVAVYYCQQYYSYPYTFGQGTKLEIKS |
| | | | | GGGGSEVQLVESGGGLVQPGGSLKLSCAASGFTFNK |
| | | | | YAMNWVRQAPGKGLEWVARIRSKYNNYATYYADSVK |
| | | | | DRFTISRDDSKNTAYLQMNNLKTEDTAVYYCVRHGN |
| | | | | FGNSYISYWAYWGQGTLVTVSSGGGGSGGGGSGGGG |
| | | | | SQTVVTQEPSLTVSPGGTVTLTCGSSTGAVTSGNYP |
| | | | | NWVQQKPGQAPRGLIGGTKFLAPGTPARFSGSLLGG |
| | | | | KAALTLSGVQPEDEAEYYCVLWYSNRWVFGGGTKLT |
| | | | | VLGGGGDKTHTCPPCPAPELLGGPSVFLFPPKPKDT |
| | | | | LMISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEM |
| | | | | TKNQVSLTCLVKGFYPSDIAVEWESNGQPENNYKTT |
| | | | | PPVLDSDGSFFLYSKLTVDKSRWQQGNVFSCSVMHE |
| | | | | ALHNHYTQKSLSLSPGKGGGGSGGGGSGGGGSGGGG |
| | | | | SGGGGSGGGGSDKTHTCPPCPAPELLGGPSVFLFPP |
| | | | | KPKDTLMISRTPEVTCVVVDVSHEDPEVKFNWYVDG |
| | | | | VEVHNAKTKPCEEQYGSTYRCVSVLTVLHQDWLNGK |
| | | | | EYKCKVSNKALPAPIEKTISKAKGQPREPQVYTLPP |
| | | | | SREEMTKNQVSLTCLVKGFYPSDIAVEWESNGQPEN |
| | | | | NYKTTPPVLDSDGSFFLYSKLTVDKSRWQQGNVFSC |
| | | | | SVMHEALHNHYTQKSLSLSPGK |
| 126. | BCMA A7 27- | artificial | aa | NHIIH |
| | C4-G7 CDR1 | | | |
| | VH | | | |
| 127. | BCMA A7 27- | artificial | aa | YINPYPGYHAYNEKFQG |
| | C4-G7 CDR2 | | | |
| | VH | | | |
| 128. | BCMA A7 27- | artificial | aa | DGYYRDTDVLDY |
| | C4-G7 CDR3 | | | |
| | VH | | | |
| 129. | BCMA A7 27- | artificial | aa | QASQDISNYLN |
| | C4-G7 CDR1 | | | |
| | VL | | | |
| 130. | BCMA A7 27- | artificial | aa | YTSRLHT |
| | C4-G7 CDR2 | | | |
| | VL | | | |
| 131. | BCMA A7 27- | artificial | aa | QQGNTLPWT |
| | C4-G7 CDR3 | | | |
| | VL | | | |
| 132. | BCMA A7 27- | artificial | aa | QVQLVQSGAEVKKPGASVKVSCKASGYTFTNHIIHW |
| | C4-G7 CC | | | VRQAPGQCLEWMGYINPYPGYHAYNEKFQGRATMTS |
| | (44/100) VH | | | DTSTSTVYMELSSLRSEDTAVYYCARDGYYRDTDVL |
| | | | | DYWGQGTLVTVSS |
| 133. | BCMA A7 27- | artificial | aa | DIQMTQSPSSLSASVGDRVTITCQASQDISNYLNWY |
| | C4-G7 CC | | | QQKPGKAPKLLIYYTSRLHTGVPSRFSGSGSGTDFT |
| | (44/100) VL | | | FTISSLEPEDIATYYCQQGNTLPWTFGCGTKLEIK |
| 134. | BCMA A7 27- | artificial | aa | QVQLVQSGAEVKKPGASVKVSCKASGYTFTNHIIHW |
| | C4-G7 CC | | | VRQAPGQCLEWMGYINPYPGYHAYNEKFQGRATMTS |
| | (44/100) scFv | | | DTSTSTVYMELSSLRSEDTAVYYCARDGYYRDTDVL |
| | | | | DYWGQGTLVTVSSGGGGSGGGGSGGGGSDIQMTQSP |
| | | | | SSLSASVGDRVTITCQASQDISNYLNWYQQKPGKAP |
| | | | | KLLIYYTSRLHTGVPSRFSGSGSGTDFTFTISSLEP |
| | | | | EDIATYYCQQGNTLPWTFGCGTKLEIK |
| 135. | BCMA A7 27- | artificial | aa | QVQLVQSGAEVKKPGASVKVSCKASGYTFTNHIIHW |
| | C4-G7 CC | | | VRQAPGQCLEWMGYINPYPGYHAYNEKFQGRATMTS |
| | (44/100) × | | | DTSTSTVYMELSSLRSEDTAVYYCARDGYYRDTDVL |
| | I2C0 bispecific | | | DYWGQGTLVTVSSGGGGSGGGGSGGGGSDIQMTQSP |
| | molecule | | | SSLSASVGDRVTITCQASQDISNYLNWYQQKPGKAP |
| | | | | KLLIYYTSRLHTGVPSRFSGSGSGTDFTFTISSLEP |
| | | | | EDIATYYCQQGNTLPWTFGCGTKLEIKSGGGGSEVQ |
| | | | | LVESGGGLVQPGGSLKLSCAASGFTFNKYAMNWVRQ |
| | | | | APGKGLEWVARIRSKYNNYATYYADSVKDRFTISRD |
| | | | | DSKNTAYLQMNNLKTEDTAVYYCVRHGNFGNSYISY |
| | | | | WAYWGQGTLVTVSSGGGGSGGGGSGGGGSQTVVTQE |
| | | | | PSLTVSPGGTVTLTCGSSTGAVTSGNYPNWVQQKPG |
| | | | | QAPRGLIGGTKFLAPGTPARFSGSLLGGKAALTLSG |
| | | | | VQPEDEAEYYCVLWYSNRWVFGGGTKLTVL |
| 136. | BCMA A7 27- | artificial | aa | QVQLVQSGAEVKKPGASVKVSCKASGYTFTNHIIHWVRQ |
| | C4-G7 CC | | | APGQCLEWMGYINPYPGYHAYNEKFQGRATMTSDTSTST |
| | (44/100) × | | | VYMELSSLRSEDTAVYYCARDGYYRDTDVLDYWGQGTLV |
| | I2C0-scFc | | | TVSSGGGGSGGGGSGGGGSDIQMTQSPSSLSASVGDRVT |
| | bispecific | | | ITCQASQDISNYLNWYQQKPGKAPKLLIYYTSRLHTGVP |
| | molecule HLE | | | SRFSGSGSGTDFTFTISSLEPEDIATYYCQQGNTLPWTF |
| | | | | GCGTKVEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGKGGGGSGGGGSGG |
| | | | | GGSGGGGSGGGGSGGGGSDKTHTCPPCPAPELLGGPSVF |
| | | | | LFPPKPKDTLMISRTPEVTCVVVDVSHEDPEVKFNWYVD |
| | | | | GVEVHNAKTKPCEEQYGSTYRCVSVLTVLHQDWLNGKEY |
| | | | | KCKVSNKALPAPIEKTISKAKGQPREPQVYTLPPSREEM |
| | | | | TKNQVSLTCLVKGFYPSDIAVEWESNGQPENNYKTTPPV |
| | | | | LDSDGSFFLYSKLTVDKSRWQQGNVFSCSVMHEALHNHY |
| | | | | TQKSLSLSPGK |
| 137. | PM 76-B10.17 | artificial | aa | DYYMY |
| | CC VH CDR1 | | | |
| 138. | PM 76-B10.17 | artificial | aa | IISDAGYYTYYSDIIKG |
| | CC VH CDR2 | | | |
| 139. | PM 76-B10.17 | artificial | aa | GFPLLRHGAMDY |
| | CC VH CDR3 | | | |
| 140. | PM 76-B10.17 | artificial | aa | KASQNVDANVA |
| | CC VL CDR1 | | | |
| 141. | PM 76-B10.17 | artificial | aa | SASYVYW |
| | CC VL CDR2 | | | |
| 142. | PM 76-B10.17 | artificial | aa | QQYDQQLIT |
| | CC VL CDR3 | | | |
| 143. | PM 76-B10.17 | artificial | aa | QVQLVESGGGLVKPGESLRLSCAASGFTFSDYYMYWVRQ |
| | CC VH | | | APGKCLEWVAIISDAGYYTYYSDIIKGRFTISRDNAKNS |
| | | | | LYLQMNSLKAEDTAVYYCARGFPLLRHGAMDYWGQGTLV |
| | | | | TVSS |
| 144. | PM 76-B10.17 | artificial | aa | DIQMTQSPSSLSASVGDRVTITCKASQNVDANVAWYQQK |
| | CC VL | | | PGQAPKSLIYSASYVYWDVPSRFSGSASGTDFTLTISSV |
| | | | | QSEDFATYYCQQYDQQLITFGCGTKLEIK |
| 145. | PM 76-B10.17 | artificial | aa | QVQLVESGGGLVKPGESLRLSCAASGFTFSDYYMYWVRQ |
| | CC scFv | | | APGKCLEWVAIISDAGYYTYYSDIIKGRFTISRDNAKNS |
| | | | | LYLQMNSLKAEDTAVYYCARGFPLLRHGAMDYWGQGTLV |
| | | | | TVSSGGGGSGGGGSGGGGSDIQMTQSPSSLSASVGDRVT |
| | | | | ITCKASQNVDANVAWYQQKPGQAPKSLIYSASYVYWDVP |
| | | | | SRFSGSASGTDFTLTISSVQSEDFATYYCQQYDQQLITF |
| | | | | VGCGTKLEIK |
| 146. | PM 76-B10.17 | artificial | aa | QVQLVESGGGLVKPGESLRLSCAASGFTFSDYYMYWVRQ |
| | CC × I2C0 | | | APGKCLEWVAIISDAGYYTYYSDIIKGRFTISRDNAKNS |
| | bispecific | | | LYLQMNSLKAEDTAVYYCARGFPLLRHGAMDYWGQGTLV |
| | molecule | | | TVSSGGGGSGGGGSGGGGSDIQMTQSPSSLSASVGDRVT |
| | | | | ITCKASQNVDANVAWYQQKPGQAPKSLIYSASYVYWDVP |
| | | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVL |
| 147. | PM 76- | artificial | aa | QVQLVESGGGLVKPGESLRLSCAASGFTFSDYYMYWVRQ |
| | B10.17 CC × | | | APGKCLEWVAIISDAGYYTYYSDIIKGRFTISRDNAKNS |
| | I2C0-scFc | | | LYLQMNSLKAEDTAVYYCARGFPLLRHGAMDYWGQGTLV |
| | bispecific | | | TVSSGGGGSGGGGSGGGGSDIQMTQSPSSLSASVGDRVT |
| | HLE molecule | | | ITCKASQNVDANVAWYQQKPGQAPKSLIYSASYVYWDVP |
| | | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGKGGGGSGGGGSGG |
| | | | | GGSGGGGSGGGGSGGGGSDKTHTCPPCPAPELLGGPSVF |
| | | | | LFPPKPKDTLMISRTPEVTCVVVDVSHEDPEVKFNWYVD |
| | | | | GVEVHNAKTKPCEEQYGSTYRCVSVLTVLHQDWLNGKEY |
| | | | | KCKVSNKALPAPIEKTISKAKGQPREPQVYTLPPSREEM |
| | | | | TKNQVSLTCLVKGFYPSDIAVEWESNGQPENNYKTTPPV |
| | | | | LDSDGSFFLYSKLTVDKSRWQQGNVFSCSVMHEALHNHY |
| | | | | TQKSLSLSPGK |
| 148. | PM 76- | artificial | aa | QVQLVESGGGLVKPGESLRLSCAASGFTFSDYYMYWVRQ |
| | B10.17 CC × | | | APGKCLEWVAIISDAGYYTYYSDIIKGRFTISRDNAKNS |
| | I2C0- | | | LYLQMNSLKAEDTAVYYCARGFPLLRHGAMDYWGQGTLV |
| | scFc_delGK | | | TVSSGGGGSGGGGSGGGGSDIQMTQSPSSLSASVGDRVT |
| | bispecific | | | ITCKASQNVDANVAWYQQKPGQAPKSLIYSASYVYWDVP |
| | HLE molecule | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGGGGSGGGGSGGGG |
| | | | | SGGGGSGGGGSGGGGSDKTHTCPPCPAPELLGGPSVFLF |
| | | | | PPKPKDTLMISRTPEVTCVVVDVSHEDPEVKFNWYVDGV |
| | | | | EVHNAKTKPCEEQYGSTYRCVSVLTVLHQDWLNGKEYKC |
| | | | | KVSNKALPAPIEKTISKAKGQPREPQVYTLPPSREEMTK |
| | | | | NQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPGK |
| 149. | PM 76-B10.17 | artificial | aa | QVQLVESGGGLVKPGESLRLSCAASGFTFSDYYMYWVRQ |
| | CC × I2C0 CC | | | APGKCLEWVAIISDAGYYTYYSDIIKGRFTISRDNAKNS |
| | (103/43)-scFc | | | LYLQMNSLKAEDTAVYYCARGFPLLRHGAMDYWGQGTLV |
| | bispecific | | | TVSSGGGGSGGGGSGGGGSDIQMTQSPSSLSASVGDRVT |
| | molecule | | | ITCKASQNVDANVAWYQQKPGQAPKSLIYSASYVYWDVP |
| | | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYCGQGTLVTVSSGGGGSGGGGSGGGGSQ |
| | | | | TVVTQEPSLTVSPGGTVTLTCGSSTGAVTSGNYPNWVQQ |
| | | | | KPGQCPRGLIGGTKFLAPGTPARFSGSLLGGKAALTLSG |
| | | | | VQPEDEAEYYCVLWYSNRWVFGGGTKLTVL |
| 150. | PM 76- | artificial | aa | QVQLVESGGGLVKPGESLRLSCAASGFTFSDYYMYWVRQ |
| | B10.17 CC × | | | APGKCLEWVAIISDAGYYTYYSDIIKGRFTISRDNAKNS |
| | I2C0 CC | | | LYLQMNSLKAEDTAVYYCARGFPLLRHGAMDYWGQGTLV |
| | (103/43)- | | | TVSSGGGGSGGGGSGGGGSDIQMTQSPSSLSASVGDRVT |
| | scFc | | | ITCKASQNVDANVAWYQQKPGQAPKSLIYSASYVYWDVP |
| | bispecific | | | SRFSGSASGTDFTLTISSVQSEDFATYYCQQYDQQLITF |
| | HLE molecule | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYCGQGTLVTVSSGGGGSGGGGSGGGGSQ |
| | | | | TVVTQEPSLTVSPGGTVTLTCGSSTGAVTSGNYPNWVQQ |
| | | | | KPGQCPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGKGGGGSGGGGSGG |
| | | | | GGSGGGGSGGGGSGGGGSDKTHTCPPCPAPELLGGPSVF |
| | | | | LFPPKPKDTLMISRTPEVTCVVVDVSHEDPEVKFNWYVD |
| | | | | GVEVHNAKTKPCEEQYGSTYRCVSVLTVLHQDWLNGKEY |
| | | | | KCKVSNKALPAPIEKTISKAKGQPREPQVYTLPPSREEM |
| | | | | TKNQVSLTCLVKGFYPSDIAVEWESNGQPENNYKTTPPV |
| | | | | LDSDGSFFLYSKLTVDKSRWQQGNVFSCSVMHEALHNHY |
| | | | | TQKSLSLSPGK |
| 151. | PM 76- | artificial | aa | QVQLVESGGGLVKPGESLRLSCAASGFTFSDYYMYWVRQ |
| | B10.17 CC × | | | APGKCLEWVAIISDAGYYTYYSDIIKGRFTISRDNAKNS |
| | I2C0 CC | | | LYLQMNSLKAEDTAVYYCARGFPLLRHGAMDYWGQGTLV |
| | (103/43)- | | | TVSSGGGGSGGGGSGGGGSDIQMTQSPSSLSASVGDRVT |
| | scFc_delGK | | | ITCKASQNVDANVAWYQQKPGQAPKSLIYSASYVYWDVP |
| | bispecific | | | SRFSGSASGTDFTLTISSVQSEDFATYYCQQYDQQLITF |
| | HLE molecule | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYCGQGTLVTVSSGGGGSGGGGSGGGGSQ |
| | | | | TVVTQEPSLTVSPGGTVTLTCGSSTGAVTSGNYPNWVQQ |
| | | | | KPGQCPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGGGGSGGGGSGGGG |
| | | | | SGGGGSGGGGSGGGGSDKTHTCPPCPAPELLGGPSVFLF |
| | | | | PPKPKDTLMISRTPEVTCVVVDVSHEDPEVKFNWYVDGV |
| | | | | EVHNAKTKPCEEQYGSTYRCVSVLTVLHQDWLNGKEYKC |
| | | | | KVSNKALPAPIEKTISKAKGQPREPQVYTLPPSREEMTK |
| | | | | NQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPGK |
| 152. | PM 76_B10.11 | artificial | aa | DYYMY |
| | CC VH CDR1 | | | |
| 153. | PM 76-B10.11 | artificial | aa | IISDGGYYTYYSDIIKG |
| | CC VH CDR2 | | | |
| 154. | PM 76_B10.11 | artificial | aa | GFPLLRHGAMDY |
| | CC VH CDR3 | | | |
| 155. | PM 76_B10.11 | artificial | aa | KASQNVDTNVA |
| | CC VL CDR1 | | | |
| 156. | PM 76_B10.11 | artificial | aa | SASYVYW |
| | CC VL CDR2 | | | |
| 157. | PM 76_B10.11 | artificial | aa | QQYDQQLIT |
| | CC VL CDR3 | | | |
| 158. | PM 76_B10.11 | artificial | aa | QVQLVESGGGLVKPGESLRLSCAASGFTFSDYYMYWVRQ |
| | CC VH | | | APGKGLEWVAIISDGGYYTYYSDIIKGRFTISRDNAKNS |
| | | | | LYLQMNSLKAEDTAVYYCARGFPLLRHGAMDYWGQGTLV |
| | | | | TVSS |
| 159. | PM 76-B10.11 | artificial | aa | DIQMTQSPSSLSASVGDRVTITCKASQNVDTNVAWYQQK |
| | CC VL | | | PGQAPKSLIYSASYVYWDVPSRFSGSASGTDFTLTISSV |
| | | | | QSEDFATYYCQQYDQQLITFGGGTKLEIK |
| 160. | PM 76-B10.11 | artificial | aa | QVQLVESGGGLVKPGESLRLSCAASGFTFSDYYMYWVRQ |
| | CC scFv | | | APGKGLEWVAIISDGGYYTYYSDIIKGRFTISRDNAKNS |
| | | | | LYLQMNSLKAEDTAVYYCARGFPLLRHGAMDYWGQGTLV |
| | | | | TVSSGGGGSGGGGSGGGGSDIQMTQSPSSLSASVGDRVT |
| | | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GGGTKLEIK |
| 161. | PM 76-B10.11 | artificial | aa | QVQLVESGGGLVKPGESLRLSCAASGFTFSDYYMYWVRQ |
| | CC × l2C0 | | | APGKGLEWVAIISDGGYYTYYSDIIKGRFTISRDNAKNS |
| | bispecific | | | LYLQMNSLKAEDTAVYYCARGFPLLRHGAMDYWGQGTLV |
| | molecule | | | TVSSGGGGSGGGGSGGGGSDIQMTQSPSSLSASVGDRVT |
| | | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GGGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVL |
| 162. | PM 76- | artificial | aa | QVQLVESGGGLVKPGESLRLSCAASGFTFSDYYMYWVRQ |
| | B10.11 CC × | | | APGKGLEWVAIISDGGYYTYYSDIIKGRFTISRDNAKNS |
| | I2C0-scFc | | | LYLQMNSLKAEDTAVYYCARGFPLLRHGAMDYWGQGTLV |
| | bispecific | | | TVSSGGGGSGGGGSGGGGSDIQMTQSPSSLSASVGDRVT |
| | HLE molecule | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GGGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGKGGGGSGGGGSGG |
| | | | | GGSGGGGSGGGGSGGGGSDKTHTCPPCPAPELLGGPSVF |
| | | | | LFPPKPKDTLMISRTPEVTCVVVDVSHEDPEVKFNWYVD |
| | | | | GVEVHNAKTKPCEEQYGSTYRCVSVLTVLHQDWLNGKEY |
| | | | | KCKVSNKALPAPIEKTISKAKGQPREPQVYTLPPSREEM |
| | | | | TKNQVSLTCLVKGFYPSDIAVEWESNGQPENNYKTTPPV |
| | | | | LDSDGSFFLYSKLTVDKSRWQQGNVFSCSVMHEALHNHY |
| | | | | TQKSLSLSPGK |
| 163. | PM 76- | artificial | aa | QVQLVESGGGLVKPGESLRLSCAASGFTFSDYYMYWVRQ |
| | B10.11 CC × | | | APGKGLEWVAIISDGGYYTYYSDIIKGRFTISRDNAKNS |
| | I2C0- | | | LYLQMNSLKAEDTAVYYCARGFPLLRHGAMDYWGQGTLV |
| | scFc_delGK | | | TVSSGGGGSGGGGSGGGGSDIQMTQSPSSLSASVGDRVT |
| | bispecific | | | ITCKASQNVDTNVAWYQQKPGQAPKSLIYSASYVYWDVP |
| | HLE molecule | | | SRFSGSASGTDFTLTISSVQSEDFATYYCQQYDQQLITF |
| | | | | GGGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGGGGSGGGGSGGGG |
| | | | | SGGGGSGGGGSGGGGSDKTHTCPPCPAPELLGGPSVFLF |
| | | | | PPKPKDTLMISRTPEVTCVVVDVSHEDPEVKFNWYVDGV |
| | | | | EVHNAKTKPCEEQYGSTYRCVSVLTVLHQDWLNGKEYKC |
| | | | | KVSNKALPAPIEKTISKAKGQPREPQVYTLPPSREEMTK |
| | | | | NQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPGK |
| 164. | PM 76_B10.11 | artificial | aa | QVQLVESGGGLVKPGESLRLSCAASGFTFSDYYMYWVRQ |
| | CC × 12C0 CC | | | APGKGLEWVAIISDGGYYTYYSDIIKGRFTISRDNAKNS |
| | (103/43)-scFc | | | LYLQMNSLKAEDTAVYYCARGFPLLRHGAMDYWGQGTLV |
| | bispecific | | | TVSSGGGGSGGGGSGGGGSDIQMTQSPSSLSASVGDRVT |
| | molecule | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GGGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYCGQGTLVTVSSGGGGSGGGGSGGGGSQ |
| | | | | TVVTQEPSLTVSPGGTVTLTCGSSTGAVTSGNYPNWVQQ |
| | | | | KPGQCPRGLIGGTKFLAPGTPARFSGSLLGGKAALTLSG |
| | | | | VQPEDEAEYYCVLWYSNRWVFGGGTKLTVL |
| 165. | PM 76- | artificial | aa | QVQLVESGGGLVKPGESLRLSCAASGFTFSDYYMYWVRQ |
| | B10.11 CC × | | | APGKGLEWVAIISDGGYYTYYSDIIKGRFTISRDNAKNS |
| | I2C0 CC | | | LYLQMNSLKAEDTAVYYCARGFPLLRHGAMDYWGQGTLV |
| | (103/43)- | | | TVSSGGGGSGGGGSGGGGSDIQMTQSPSSLSASVGDRVT |
| | scFc | | | ITCKASQNVDTNVAWYQQKPGQAPKSLIYSASYVYWDVP |
| | bispecific | | | SRFSGSASGTDFTLTISSVQSEDFATYYCQQYDQQLITF |
| | HLE molecule | | | GGGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYCGQGTLVTVSSGGGGSGGGGSGGGGSQ |
| | | | | TVVTQEPSLTVSPGGTVTLTCGSSTGAVTSGNYPNWVQQ |
| | | | | KPGQCPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGKGGGGSGGGGSGG |
| | | | | GGSGGGGSGGGGSGGGGSDKTHTCPPCPAPELLGGPSVF |
| | | | | LFPPKPKDTLMISRTPEVTCVVVDVSHEDPEVKFNWYVD |
| | | | | GVEVHNAKTKPCEEQYGSTYRCVSVLTVLHQDWLNGKEY |
| | | | | KCKVSNKALPAPIEKTISKAKGQPREPQVYTLPPSREEM |
| | | | | TKNQVSLTCLVKGFYPSDIAVEWESNGQPENNYKTTPPV |
| | | | | LDSDGSFFLYSKLTVDKSRWQQGNVFSCSVMHEALHNHY |
| | | | | TQKSLSLSPGK |
| 166. | PM 76- | artificial | aa | QVQLVESGGGLVKPGESLRLSCAASGFTFSDYYMYWVRQ |
| | B10.11 CC × | | | APGKGLEWVAIISDGGYYTYYSDIIKGRFTISRDNAKNS |
| | I2C0 CC | | | LYLQMNSLKAEDTAVYYCARGFPLLRHGAMDYWGQGTLV |
| | (103/43)- | | | TVSSGGGGSGGGGSGGGGSDIQMTQSPSSLSASVGDRVT |
| | scFc_delGK | | | ITCKASQNVDTNVAWYQQKPGQAPKSLIYSASYVYWDVP |
| | bispecific | | | SRFSGSASGTDFTLTISSVQSEDFATYYCQQYDQQLITF |
| | HLE molecule | | | GGGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYCGQGTLVTVSSGGGGSGGGGSGGGGSQ |
| | | | | TVVTQEPSLTVSPGGTVTLTCGSSTGAVTSGNYPNWVQQ |
| | | | | KPGQCPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGGGGSGGGGSGGGG |
| | | | | SGGGGSGGGGSGGGGSDKTHTCPPCPAPELLGGPSVFLF |
| | | | | PPKPKDTLMISRTPEVTCVVVDVSHEDPEVKFNWYVDGV |
| | | | | EVHNAKTKPCEEQYGSTYRCVSVLTVLHQDWLNGKEYKC |
| | | | | KVSNKALPAPIEKTISKAKGQPREPQVYTLPPSREEMTK |
| | | | | NQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPGK |
| 167. | PM 76-B10.11 | artificial | aa | DYYMY |
| | CC × I2C0-scFc | | | |
| | VH CDR1 | | | |
| 168. | PM 76-B10.11 | artificial | aa | IISDGGYYTYYSDIIKG |
| | CC × I2C0-scFc | | | |
| | VH CDR2 | | | |
| 169. | PM 76-B10.11 | artificial | aa | GFPLLRHGAMDY |
| | CC × I2C0-scFc | | | |
| | VH CDR3 | | | |
| 170. | PM 76-B10.11 | artificial | aa | KASQNVDTNVA |
| | CC × I2C0-scFc | | | |
| | VL CDR1 | | | |
| 171. | PM 76-B10.11 | artificial | aa | SASYVYW |
| | CC × I2C0-scFc | | | |
| | VL CDR2 | | | |
| 172. | PM 76-B10.11 | artificial | aa | QQYDQQLIT |
| | CC × I2C0-scFc | | | |
| | VL CDR3 | | | |
| 173. | PM 76-B10.11 | artificial | aa | QVQLVESGGGLVKPGESLRLSCAASGFTFSDYYMYWVRQ |
| | CC × I2C0-scFc | | | APGKCLEWVAIISDGGYYTYYSDIIKGRFTISRDNAKNS |
| | VH | | | LYLQMNSLKAEDTAVYYCARGFPLLRHGAMDYWGQGTLV |
| | | | | TVSS |
| 174. | PM 76-B10.11 | artificial | aa | DIQMTQSPSSLSASVGDRVTITCKASQNVDTNVAWYQQK |
| | CC × I2C0-scFc | | | PGQAPKSLIYSASYVYWDVPSRFSGSASGTDFTLTISSV |
| | VL | | | QSEDFATYYCQQYDQQLITFGCGTKLEIK |
| 175. | PM 76-1310.11 | artificial | aa | QVQLVESGGGLVKPGESLRLSCAASGFTFSDYYMYWVRQ |
| | CC × I2C0-scFc | | | APGKCLEWVAIISDGGYYTYYSDIIKGRFTISRDNAKNS |
| | scFv | | | LYLQMNSLKAEDTAVYYCARGFPLLRHGAMDYWGQGTLV |
| | | | | TVSSGGGGSGGGGSGGGGSDIQMTQSPSSLSASVGDRVT |
| | | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GCGTKLEIK |
| 176. | PM 76-B10.11 | artificial | aa | QVQLVESGGGLVKPGESLRLSCAASGFTFSDYYMYWVRQ |
| | CC × I2C0-scFc | | | APGKCLEWVAIISDGGYYTYYSDIIKGRFTISRDNAKNS |
| | bispecific | | | LYLQMNSLKAEDTAVYYCARGFPLLRHGAMDYWGQGTLV |
| | molecule | | | TVSSGGGGSGGGGSGGGGSDIQMTQSPSSLSASVGDRVT |
| | | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVL |
| 177. | PM 76-B10.11 | artificial | aa | QVQLVESGGGLVKPGESLRLSCAASGFTFSDYYMYWVRQ |
| | CC × I2C0-scFc | | | APGKCLEWVAIISDGGYYTYYSDIIKGRFTISRDNAKNS |
| | bispecific | | | LYLQMNSLKAEDTAVYYCARGFPLLRHGAMDYWGQGTLV |
| | HLE molecule | | | TVSSGGGGSGGGGSGGGGSDIQMTQSPSSLSASVGDRVT |
| | | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGKGGGGSGGGGSGG |
| | | | | GGSGGGGSGGGGSGGGGSDKTHTCPPCPAPELLGGPSVF |
| | | | | LFPPKPKDTLMISRTPEVTCVVVDVSHEDPEVKFNWYVD |
| | | | | GVEVHNAKTKPCEEQYGSTYRCVSVLTVLHQDWLNGKEY |
| | | | | KCKVSNKALPAPIEKTISKAKGQPREPQVYTLPPSREEM |
| | | | | TKNQVSLTCLVKGFYPSDIAVEWESNGQPENNYKTTPPV |
| | | | | LDSDGSFFLYSKLTVDKSRWQQGNVFSCSVMHEALHNHY |
| | | | | TQKSLSLSPGK |
| 178. | PM 76-B10.11 | artificial | aa | QVQLVESGGGLVKPGESLRLSCAASGFTFSDYYMYWVRQ |
| | CC × I2C0- | | | APGKCLEWVAIISDGGYYTYYSDIIKGRFTISRDNAKNS |
| | scFc_delGK | | | LYLQMNSLKAEDTAVYYCARGFPLLRHGAMDYWGQGTLV |
| | bispecific | | | TVSSGGGGSGGGGSGGGGSDIQMTQSPSSLSASVGDRVT |
| | HLE molecule | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYWGQGTLVTVSSGGGGSGGGGSGGGGSQ |
| | | | | TVVTQEPSLTVSPGGTVTLTCGSSTGAVTSGNYPNWVQQ |
| | | | | KPGQAPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGGGGSGGGGSGGGG |
| | | | | SGGGGSGGGGSGGGGSDKTHTCPPCPAPELLGGPSVFLF |
| | | | | PPKPKDTLMISRTPEVTCVVVDVSHEDPEVKFNWYVDGV |
| | | | | EVHNAKTKPCEEQYGSTYRCVSVLTVLHQDWLNGKEYKC |
| | | | | KVSNKALPAPIEKTISKAKGQPREPQVYTLPPSREEMTK |
| | | | | NQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPGK |
| 179. | PM 76-B10.11 | artificial | aa | QVQLVESGGGLVKPGESLRLSCAASGFTFSDYYMYWVRQ |
| | CC × l2C0 CC | | | APGKCLEWVAIISDGGYYTYYSDIIKGRFTISRDNAKNS |
| | (103/43)-scFc | | | LYLQMNSLKAEDTAVYYCARGFPLLRHGAMDYWGQGTLV |
| | bispecific | | | TVSSGGGGSGGGGSGGGGSDIQMTQSPSSLSASVGDRVT |
| | molecule | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYCGQGTLVTVSSGGGGSGGGGSGGGGSQ |
| | | | | TVVTQEPSLTVSPGGTVTLTCGSSTGAVTSGNYPNWVQQ |
| | | | | KPGQCPRGLIGGTKFLAPGTPARFSGSLLGGKAALTLSG |
| | | | | VQPEDEAEYYCVLWYSNRWVFGGGTKLTVL |
| 180. | PM 76-B10.11 | artificial | aa | QVQLVESGGGLVKPGESLRLSCAASGFTFSDYYMYWVRQ |
| | CC × l2C0 CC | | | APGKCLEWVAIISDGGYYTYYSDIIKGRFTISRDNAKNS |
| | (103/43)-scFc | | | LYLQMNSLKAEDTAVYYCARGFPLLRHGAMDYWGQGTLV |
| | bispecific | | | TVSSGGGGSGGGGSGGGGSDIQMTQSPSSLSASVGDRVT |
| | HLE molecule | | | ITCKASQNVDTNVAWYQQKPGQAPKSLIYSASYVYWDVP |
| | | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYCGQGTLVTVSSGGGGSGGGGSGGGGSQ |
| | | | | TVVTQEPSLTVSPGGTVTLTCGSSTGAVTSGNYPNWVQQ |
| | | | | KPGQCPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGKGGGGSGGGGSGG |
| | | | | GGSGGGGSGGGGSGGGGSDKTHTCPPCPAPELLGGPSVF |
| | | | | LFPPKPKDTLMISRTPEVTCVVVDVSHEDPEVKFNWYVD |
| | | | | GVEVHNAKTKPCEEQYGSTYRCVSVLTVLHQDWLNGKEY |
| | | | | KCKVSNKALPAPIEKTISKAKGQPREPQVYTLPPSREEM |
| | | | | TKNQVSLTCLVKGFYPSDIAVEWESNGQPENNYKTTPPV |
| | | | | LDSDGSFFLYSKLTVDKSRWQQGNVFSCSVMHEALHNHY |
| | | | | TQKSLSLSPGK |
| 181. | PM 76-B10.11 | artificial | aa | QVQLVESGGGLVKPGESLRLSCAASGFTFSDYYMYWVRQ |
| | CC × l2C0 CC | | | APGKCLEWVAIISDGGYYTYYSDIIKGRFTISRDNAKNS |
| | (103/43)- | | | LYLQMNSLKAEDTAVYYCARGFPLLRHGAMDYWGQGTLV |
| | scFc_delGK | | | TVSSGGGGSGGGGSGGGGSDIQMTQSPSSLSASVGDRVT |
| | bispecific | | | ITCKASQNVDTNVAWYQQKPGQAPKSLIYSASYVYWDVP |
| | HLE molecule | | | SRFSGSASGTDFTLTISSVQSEDFATYYCQQYDQQLITF |
| | | | | GCGTKLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHG |
| | | | | NFGNSYISYWAYCGQGTLVTVSSGGGGSGGGGSGGGGSQ |
| | | | | TVVTQEPSLTVSPGGTVTLTCGSSTGAVTSGNYPNWVQQ |
| | | | | KPGQCPRGLIGGTKFLAPGTPARFSGSLLGGKAALTLSG |
| | | | | VQPEDEAEYYCVLWYSNRWVFGGGTKLTVLGGGGDKTHT |
| | | | | CPPCPAPELLGGPSVFLFPPKPKDTLMISRTPEVTCVVV |
| | | | | DVSHEDPEVKFNWYVDGVEVHNAKTKPCEEQYGSTYRCV |
| | | | | SVLTVLHQDWLNGKEYKCKVSNKALPAPIEKTISKAKGQ |
| | | | | PREPQVYTLPPSREEMTKNQVSLTCLVKGFYPSDIAVEW |
| | | | | ESNGQPENNYKTTPPVLDSDGSFFLYSKLTVDKSRWQQG |
| | | | | NVFSCSVMHEALHNHYTQKSLSLSPGGGGSGGGGSGGGG |
| | | | | SGGGGSGGGGSGGGGSDKTHTCPPCPAPELLGGPSVFLF |
| | | | | PPKPKDTLMISRTPEVTCVVVDVSHEDPEVKFNWYVDGV |
| | | | | EVHNAKTKPCEEQYGSTYRCVSVLTVLHQDWLNGKEYKC |
| | | | | KVSNKALPAPIEKTISKAKGQPREPQVYTLPPSREEMTK |
| | | | | NQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPGK |
| 182. | IgG1 hinge | artificial | aa | DKTHTCPPCP |
| 183. | IgG2 subtype | artificial | aa | ERKCCVECPPCP |
| | hinge | | | |
| 184. | IgG3 subtype | artificial | aa | ELKTPLDTTHTCPRCP |
| | hinge | | | |
| 185. | IgG3 subtype | artificial | aa | ELKTPLGDTTHTCPRCP |
| | hinge | | | |
| 186. | IgG4 subtype | artificial | aa | ESKYGPPCPSCP |
| | hinge | | | |
| 187. | G4S linker | artificial | aa | GGGGS |
| 188. | (G4S)2 linker | artificial | aa | GGGGSGGGGS |
| 189. | (G4S)3 linker | artificial | aa | GGGGSGGGGSGGGGS |
| 190. | (G4S)4 linker | artificial | aa | GGGGSGGGGSGGGGSGGGGS |
| 191. | (G4S)5 linker | artificial | aa | GGGGSGGGGSGGGGSGGGGSGGGGS |
| 192. | (G4S)6 linker | artificial | aa | GGGGSGGGGSGGGGSGGGGSGGGGSGGGGS |
| 193. | (G4S)7 linker | artificial | aa | GGGGSGGGGSGGGGSGGGGSGGGGSGGGGSGGGGS |
| 194. | (G4S)8 linker | artificial | aa | GGGGSGGGGSGGGGSGGGGSGGGGSGGGGSGGGGSGGGG |
| | | | | S |
| 195. | Peptide linker | artificial | aa | PGGGGS |
| 196. | Peptide linker | artificial | aa | PGGDGS |
| 197. | Peptide linker | artificial | aa | SGGGGS |
| 198. | Peptide linker | artificial | aa | GGGG |
| 199. | hexa-histidine | artificial | aa | HHHHHH |
| | tag | | | |
| 200. | CD3e binder | artificial | aa | QTVVTQEPSLTVSPGGTVTLTCGSSTGAVTSGNYPNWVQ |
| | VL | | | QKPGQAPRGLIGGTKFLAPGTPARFSGSLLGGKAALTLS |
| | | | | GVQPEDEAEYYCVLWYSNRWVFGGGTKLTVL |
| 201. | CD3e binder | artificial | aa | EVQLVESGGGLVQPGGSLRLSCAASGFTFNSYAMNWVRQ |
| | VH | | | APGKGLEWVARIRSKYNNYATYYADSVKGRFTISRDDSK |
| | | | | NTAYLQMNSLKTEDTAVYYCVRHGNFGNSYVSWWAYWGQ |
| | | | | GTLVTVSS |
| 202. | CD3e binder | artificial | aa | EVQLVESGGGLVQPGGSLRLSCAASGFTFNSYAMNWVRQ |
| | scFv | | | APGKGLEWVARIRSKYNNYATYYADSVKGRFTISRDDSK |
| | | | | NTAYLQMNSLKTEDTAVYYCVRHGNFGNSYVSWWAYWGQ |
| | | | | GTLVTVSSGGGGSGGGGSGGGGSQTVVTQEPSLTVSPGG |
| | | | | TVTLTCGSSTGAVTSGNYPNWVQQKPGQAPRGLIGGTKF |
| | | | | LAPGTPARFSGSLLGGKAALTLSGVQPEDEAEYYCVLWY |
| | | | | SNRWVFGGGTKLTVL |
| 203. | Fc monomer- | artificial | aa | DKTHTCPPCPAPELLGGPSVFLFPPKPKDTLMISRTPEV |
| | 1 | | | TCVVVDVSHEDPEVKFNWYVDGVEVHNAKTKPCEEQYGS |
| | +c/−g | | | TYRCVSVLTVLHQDWLNGKEYKCKVSNKALPAPIEKTIS |
| | | | | KAKGQPREPQVYTLPPSREEMTKNQVSLTCLVKGFYPSD |
| | | | | IAVEWESNGQPENNYKTTPPVLDSDGSFFLYSKLTVDKS |
| | | | | RWQQGNVFSCSVMHEALHNHYTQKSLSLSPGK |
| 204. | Fc monomer- | artificial | aa | DKTHTCPPCPAPELLGGPSVFLFPPKPKDTLMISRTPEV |
| | 2 | | | TCVVVDVSHEDPEVKFNWYVDGVEVHNAKTKPCEEQYGS |
| | +c/−g/delGK | | | TYRCVSVLTVLHQDWLNGKEYKCKVSNKALPAPIEKTIS |
| | | | | KAKGQPREPQVYTLPPSREEMTKNQVSLTCLVKGFYPSD |
| | | | | IAVEWESNGQPENNYKTTPPVLDSDGSFFLYSKLTVDKS |
| | | | | RWQQGNVFSCSVMHEALHNHYTQKSLSLSP |
| 205. | Fc monomer- | artificial | aa | DKTHTCPPCPAPELLGGPSVFLFPPKPKDTLMISRTPEV |
| | 3 | | | TCVVVDVSHEDPEVKFNWYVDGVEVHNAKTKPREEQYNS |
| | −c/+g | | | TYRVVSVLTVLHQDWLNGKEYKCKVSNKALPAPIEKTIS |
| | | | | KAKGQPREPQVYTLPPSREEMTKNQVSLTCLVKGFYPSD |
| | | | | IAVEWESNGQPENNYKTTPPVLDSDGSFFLYSKLTVDKS |
| | | | | RWQQGNVFSCSVMHEALHNHYTQKSLSLSPGK |
| 206. | Fc monomer- | artificial | aa | DKTHTCPPCPAPELLGGPSVFLFPPKPKDTLMISRTPEV |
| | 4 | | | TCVVVDVSHEDPEVKFNWYVDGVEVHNAKTKPREEQYNS |
| | −c/+g/delGK | | | TYRVVSVLTVLHQDWLNGKEYKCKVSNKALPAPIEKTIS |
| | | | | KAKGQPREPQVYTLPPSREEMTKNQVSLTCLVKGFYPSD |
| | | | | IAVEWESNGQPENNYKTTPPVLDSDGSFFLYSKLTVDKS |
| | | | | RWQQGNVFSCSVMHEALHNHYTQKSLSLSP |
| 207. | Fc monomer- | artificial | aa | DKTHTCPPCPAPELLGGPSVFLFPPKPKDTLMISRTPEV |
| | 5 | | | TCVVVDVSHEDPEVKFNWYVDGVEVHNAKTKPREEQYGS |
| | −c/−g | | | TYRVVSVLTVLHQDWLNGKEYKCKVSNKALPAPIEKTIS |
| | | | | KAKGQPREPQVYTLPPSREEMTKNQVSLTCLVKGFYPSD |
| | | | | IAVEWESNGQPENNYKTTPPVLDSDGSFFLYSKLTVDKS |
| | | | | RWQQGNVFSCSVMHEALHNHYTQKSLSLSPGK |
| 208. | Fc monomer- | artificial | aa | DKTHTCPPCPAPELLGGPSVFLFPPKPKDTLMISRTPEV |
| | 6 | | | TCVVVDVSHEDPEVKFNWYVDGVEVHNAKTKPREEQYGS |
| | −c/−g/delGK | | | TYRVVSVLTVLHQDWLNGKEYKCKVSNKALPAPIEKTIS |
| | | | | KAKGQPREPQVYTLPPSREEMTKNQVSLTCLVKGFYPSD |
| | | | | IAVEWESNGQPENNYKTTPPVLDSDGSFFLYSKLTVDKS |
| | | | | RWQQGNVFSCSVMHEALHNHYTQKSLSLSP |
| 209. | Fc monomer- | artificial | aa | DKTHTCPPCPAPELLGGPSVFLFPPKPKDTLMISRTPEV |
| | 7 | | | TCVVVDVSHEDPEVKFNWYVDGVEVHNAKTKPCEEQYNS |
| | +c/+g | | | TYRCVSVLTVLHQDWLNGKEYKCKVSNKALPAPIEKTIS |
| | | | | KAKGQPREPQVYTLPPSREEMTKNQVSLTCLVKGFYPSD |
| | | | | IAVEWESNGQPENNYKTTPPVLDSDGSFFLYSKLTVDKS |
| | | | | RWQQGNVFSCSVMHEALHNHYTQKSLSLSPGK |
| 210. | Fc monomer- | artificial | aa | DKTHTCPPCPAPELLGGPSVFLFPPKPKDTLMISRTPEV |
| | 8 | | | TCVVVDVSHEDPEVKFNWYVDGVEVHNAKTKPCEEQYNS |
| | +c/+g/delGK | | | TYRCVSVLTVLHQDWLNGKEYKCKVSNKALPAPIEKTIS |
| | | | | KAKGQPREPQVYTLPPSREEMTKNQVSLTCLVKGFYPSD |
| | | | | IAVEWESNGQPENNYKTTPPVLDSDGSFFLYSKLTVDKS |
| | | | | RWQQGNVFSCSVMHEALHNHYTQKSLSLSP |

1. A method for purifying a bispecific antigen-binding polypeptide comprising a first domain which binds to a cell surface antigen, and a second domain which binds to an extracellular epitope of the human and the Macaca CD3ε chain, wherein the method comprises the steps of
(a) providing a separation resin comprising a polymer matrix part and a ligand part, wherein the matrix part comprises a polymer, preferably polymethacrylate, and has a particle size of at least 10 μm, preferably of at least 20 μm, more preferably about 30 to 60 μm, wherein the ligand part comprises recombinant protein L, and wherein the ligand part's protein L is covalently bound to the matrix part's particles,
(b) contacting a process fluid comprising the bispecific antigen-binding polypeptide with the separation resin,
(c) capturing the bispecific antigen-binding polypeptide by the ligand part of the separation resin, wherein the bispecific antigen-binding polypeptide reversibly binds to the ligand part of the separation resin, and wherein the remainder of the process fluid does not bind to the ligand part of the separation resin,
(c) washing the bound bispecific antigen-binding polypeptide with a wash buffer which does not elute the bispecific antigen-binding polypeptide from the ligand portion, and
(d) elute the bispecific antigen-binding polypeptide from the ligand part with an elution buffer at an acidic pH.

2. The method according to claim 1, wherein the matrix part has a particle size of about 45 μm.

3. The method according to claim 1, wherein the recombinant protein L comprises a modified B4 domain with an alkali-stable tetramer ligand having multiple coupling sites.

4. The method according to claim 1, wherein the recombinant protein L reversibly binds to a bispecific antigen-binding polypeptide's κ-light chain outside of the antigen binding site.

5. The method according to claim 1, wherein the process fluid is passed through the separation resin at least one time (purification cycle) allowing the bispecific antigen-binding polypeptide to contact with the protein L (residence time), wherein bispecific antigen-binding polypeptide residence time before elution is at least about 2 minutes, preferably about 2.5 to 4 minutes.

6. The method according to claim 1, wherein the wash buffer comprises at least one of the compound selected from the group consisting of phosphate buffered saline (PBS) preferably in the range of 0.01 to 1 times concentration, 3-(N-morpholino)propanesulfonic acid (MOPS) preferably in the range of 0 to 30 mM, NaCl preferably in the range of 50 to 150 mM, Tris preferably in the range 15 to 35 mM, Arginine preferably in the range 0.25 to 1 M, and Acetate preferably in the range 40-60 mM, wherein the wash puffer is in the range of pH 5 to 8.

7. The method according to claim 1, wherein the elution buffer comprises at least one of the compound selected from the group consisting of Tris preferably in the range of 15 to 35 mM, Arginine preferably in the range of 0.25 to 1 M, Glycine preferably in the range of 50 to 150 mM and Acetate preferably in the range of 50 to 150 mM, wherein the elution buffer has a pH in the range of about 3 to 7.5, preferably pH 3.3 to 4.2.

8. The method according to claim 1, wherein the dynamic loading capacity is at least 10 mg/ml resin, preferably at least 15 mg/ml resin, more preferably at least 18 mg/ml resin.

9. The method according to claim 1, wherein the elution binding capacity is at least 7.5 mg/ml resin, preferably at least 9 mg/ml resin, more preferably 16 mg/ml resin.

10. The bispecific antigen-binding polypeptide of claim 1, wherein the antigen-binding polypeptide is a single chain antigen-binding polypeptide.

11. The bispecific antigen-binding polypeptide of claim 1 further comprising a third domain which comprises two polypeptide monomers, each comprising a hinge, a CH2 domain and a CH3 domain, wherein said two polypeptide monomers are fused to each other via a peptide linker.

12. The bispecific antigen-binding polypeptide of claim 11, wherein said third domain comprises in an amino to carboxyl order:
hinge-CH2-CH3-linker-hinge-CH2-CH3.

13. The bispecific antigen-binding polypeptide of any claim 11, wherein each of said polypeptide monomers in the third domain has an amino acid sequence that is at least 90% identical to a sequence selected from the group from the group consisting of: SEQ ID NO: 203-210.

14. The bispecific antigen-binding polypeptide of claim 11, wherein each of said polypeptide monomers has an amino acid sequence selected from SEQ ID NO: 203-210.

15. The bispecific antigen-binding polypeptide of claim 12, wherein the CH2 domain comprises an intra domain cysteine disulfide bridge.

16. The bispecific antigen-binding polypeptide of claim 1, wherein
(i) the first domain comprises two antibody variable domains and the second domain comprises two antibody variable domains;
(ii) the first domain comprises one antibody variable domain and the second domain comprises two antibody variable domains;
(iii) the first domain comprises two antibody variable domains and the second domain comprises one antibody variable domain; or
(iv) the first domain comprises one antibody variable domain and the second domain comprises one antibody variable domain.

17. The bispecific antigen-binding polypeptide of claim 1, wherein the first and second domain are fused to the third domain via a peptide linker.

18. The bispecific antigen-binding polypeptide of claim 1, wherein the polypeptide comprises in an amino to carboxyl order:
(a) the first domain;
(b) a peptide linker preferably having an amino acid sequence selected from the group consisting of SEQ ID NOs: 187-189;
(c) the second domain.

19. The bispecific antigen-binding polypeptide according to claim 17, wherein the polypeptide further comprises in an amino to carboxyl order:
(d) a peptide linker having an amino acid sequence selected from the group consisting of SEQ ID NOs: 187, 188, 189, 195, 196, 197, and 198,
(e) the first polypeptide monomer of the third domain;
(f) a peptide linker having an amino acid sequence selected from the group consisting of SEQ ID NOs: 191, 192, 193 and 194; and
(g) the second polypeptide monomer of the third domain.

20. The bispecific antigen-binding polypeptide of claim 1, wherein the first domain of the polypeptide binds to an epitope of CD33, CD19, BCMA, PSMA, MSLN, EGFRvIII, MUC17, CD70 or EpCAM, preferably CD33.

21. The bispecific antigen-binding polypeptide of claim 1, wherein the first binding domain comprises a VH region comprising CDR-H 1, CDR-H2 and CDR-H3 selected from:
(a) CDR-H1 as depicted in SEQ ID NO: 1, CDR-H2 as depicted in SEQ ID NO: 2, CDR-H3 as depicted in SEQ ID NO: 3, CDR-L1 as depicted in SEQ ID NO: 4, CDR-L2 as depicted in SEQ ID NO: 5 and CDR-L3 as depicted in SEQ ID NO: 6,
(b) CDR-H1 as depicted in SEQ ID NO: 29, CDR-H2 as depicted in SEQ ID NO: 30, CDR-H3 as depicted in SEQ ID NO: 31, CDR-L1 as depicted in SEQ ID NO: 34, CDR-L2 as depicted in SEQ ID NO: 35 and CDR-L3 as depicted in SEQ ID NO: 36,
(c) CDR-H1 as depicted in SEQ ID NO: 42, CDR-H2 as depicted in SEQ ID NO: 43, CDR-H3 as depicted in SEQ ID NO: 44, CDR-L1 as depicted in SEQ ID NO: 45, CDR-L2 as depicted in SEQ ID NO: 46 and CDR-L3 as depicted in SEQ ID NO: 47,
(d) CDR-H1 as depicted in SEQ ID NO: 53, CDR-H2 as depicted in SEQ ID NO: 54, CDR-H3 as depicted in SEQ ID NO: 55, CDR-L1 as depicted in SEQ ID NO: 56, CDR-L2 as depicted in SEQ ID NO: 57 and CDR-L3 as depicted in SEQ ID NO: 58,
(e) CDR-H1 as depicted in SEQ ID NO: 65, CDR-H2 as depicted in SEQ ID NO: 66, CDR-H3 as depicted in SEQ ID NO: 67, CDR-L1 as depicted in SEQ ID NO: 68, CDR-L2 as depicted in SEQ ID NO: 69 and CDR-L3 as depicted in SEQ ID NO: 70,
(f) CDR-H1 as depicted in SEQ ID NO: 83, CDR-H2 as depicted in SEQ ID NO: 84, CDR-H3 as depicted in SEQ ID NO: 85, CDR-L1 as depicted in SEQ ID NO: 86, CDR-L2 as depicted in SEQ ID NO: 87 and CDR-L3 as depicted in SEQ ID NO: 88,
(g) CDR-H1 as depicted in SEQ ID NO: 94, CDR-H2 as depicted in SEQ ID NO: 95, CDR-H3 as depicted in SEQ ID NO: 96, CDR-L1 as depicted in SEQ ID NO: 97, CDR-L2 as depicted in SEQ ID NO: 98 and CDR-L3 as depicted in SEQ ID NO: 99,
(h) CDR-H1 as depicted in SEQ ID NO: 105, CDR-H2 as depicted in SEQ ID NO: 106, CDR-H3 as depicted in SEQ ID NO: 107, CDR-L1 as depicted in SEQ ID NO: 109, CDR-L2 as depicted in SEQ ID NO: 110 and CDR-L3 as depicted in SEQ ID NO: 111,
(i) CDR-H1 as depicted in SEQ ID NO: 115, CDR-H2 as depicted in SEQ ID NO: 116, CDR-H3 as depicted in SEQ ID NO: 117, CDR-L1 as depicted in SEQ ID NO: 118, CDR-L2 as depicted in SEQ ID NO: 119 and CDR-L3 as depicted in SEQ ID NO: 120,
(j) CDR-H1 as depicted in SEQ ID NO: 126, CDR-H2 as depicted in SEQ ID NO: 127, CDR-H3 as depicted in SEQ ID NO: 128, CDR-L1 as depicted in SEQ ID NO: 129, CDR-L2 as depicted in SEQ ID NO: 130 and CDR-L3 as depicted in SEQ ID NO: 131,
(k) CDR-H1 as depicted in SEQ ID NO: 137, CDR-H2 as depicted in SEQ ID NO: 138, CDR-H3 as depicted in SEQ ID NO: 139, CDR-L1 as depicted in SEQ ID NO: 140, CDR-L2 as depicted in SEQ ID NO: 141 and CDR-L3 as depicted in SEQ ID NO: 142,
(l) CDR-H1 as depicted in SEQ ID NO: 152, CDR-H2 as depicted in SEQ ID NO: 153, CDR-H3 as depicted in SEQ ID NO: 154, CDR-L1 as depicted in SEQ ID NO: 155, CDR-L2 as depicted in SEQ ID NO: 156 and CDR-L3 as depicted in SEQ ID NO: 157, and
(m) CDR-H1 as depicted in SEQ ID NO: 167, CDR-H2 as depicted in SEQ ID NO: 168, CDR-H3 as depicted in SEQ ID NO: 169, CDR-L1 as depicted in SEQ ID NO: 170, CDR-L2 as depicted in SEQ ID NO: 171 and CDR-L3 as depicted in SEQ ID NO: 172.

22. A pharmaceutical composition comprising the bispecific antigen-binding polypeptide of claims 1 to 21

23. The antigen-binding polypeptide of claims 1 to 21 for use in the prevention, treatment or amelioration of a disease selected from a proliferative disease, a tumorous disease, cancer or an immunological disorder.

24. A method for improving the yield of a production process for a bispecific antigen-binding polypeptide, wherein in downstream processing the method according to claim 1 is applied.


SEQUENCE LISTING (210 sequences)

SEQ ID NO: 1 - protein, 15 aa, Artificial Sequence
  Synthetic Polypeptide
KASQSVDYDGDSYLN

SEQ ID NO: 2 - protein, 7 aa, Artificial Sequence
  Synthetic Polypeptide
DASNLVS

SEQ ID NO: 3 - protein, 9 aa, Artificial Sequence
  Synthetic Polypeptide
QQSTEDPWT

SEQ ID NO: 4 - protein, 5 aa, Artificial Sequence
  Synthetic Polypeptide
SYWMN

SEQ ID NO: 5 - protein, 17 aa, Artificial Sequence
  Synthetic Polypeptide
QIWPGDGDTNYNGKFKG

SEQ ID NO: 6 - protein, 15 aa, Artificial Sequence
  Synthetic Polypeptide
RETTTVGRYYYAMDY

SEQ ID NO: 7 - protein, 111 aa, Artificial Sequence
  Synthetic Polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 8 - protein, 124 aa, Artificial Sequence
  Synthetic Polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 9 - protein, 5 aa, Artificial Sequence
  Synthetic Polypeptide
RYTMH

SEQ ID NO: 10 - protein, 17 aa, Artificial Sequence
  Synthetic Polypeptide
YINPSRGYTNYNQKFKD

SEQ ID NO: 11 - protein, 10 aa, Artificial Sequence
  Synthetic Polypeptide
YYDDHYCLDY

SEQ ID NO: 12 - protein, 10 aa, Artificial Sequence
  Synthetic Polypeptide
RASSSVSYMN

SEQ ID NO: 13 - protein, 7 aa, Artificial Sequence
  Synthetic Polypeptide
DTSKVAS

SEQ ID NO: 14 - protein, 9 aa, Artificial Sequence
  Synthetic Polypeptide
QQWSSNPLT

SEQ ID NO: 15 - protein, 119 aa, Artificial Sequence
  Synthetic Polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 16 - protein, 108 aa, Artificial Sequence
  Synthetic Polypeptide
VDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 17 - protein, 504 aa, Artificial Sequence
  Synthetic Polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 18 - protein, 14 aa, Artificial Sequence
  Synthetic Polypeptide
GSSTGAVTSGNYPN

SEQ ID NO: 19 - protein, 7 aa, Artificial Sequence
  Synthetic Polypeptide
GTKFLAP

SEQ ID NO: 20 - protein, 9 aa, Artificial Sequence
  Synthetic Polypeptide
VLWYSNRWV

SEQ ID NO: 21 - protein, 5 aa, Artificial Sequence
  Synthetic Polypeptide
KYAMN

SEQ ID NO: 22 - protein, 19 aa, Artificial Sequence
  Synthetic Polypeptide
RIRSKYNNYATYYADSVKD

SEQ ID NO: 23 - protein, 14 aa, Artificial Sequence
  Synthetic Polypeptide
HGNFGNSYISYWAY

SEQ ID NO: 24 - protein, 125 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 25 - protein, 109 aa, Artificial Sequence
  Synthetic Polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 26 - protein, 249 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 27 - protein, 122 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 28 - protein, 122 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 29 - protein, 5 aa, Artificial Sequence
  Synthetic Polypeptide
NYGMN

SEQ ID NO: 30 - protein, 17 aa, Artificial Sequence
  Synthetic Polypeptide
WINTYTGEPTYADKFQG

SEQ ID NO: 31 - protein, 13 aa, Artificial Sequence
  Synthetic Polypeptide
WSWSDGYYVYFDY

SEQ ID NO: 32 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 33 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 34 - protein, 17 aa, Artificial Sequence
  Synthetic Polypeptide
KSSQSVLDSSTNKNSLA

SEQ ID NO: 35 - protein, 7 aa, Artificial Sequence
  Synthetic Polypeptide
WASTRES

SEQ ID NO: 36 - protein, 9 aa, Artificial Sequence
  Synthetic Polypeptide
QQSAHFPIT

SEQ ID NO: 37 - protein, 250 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 38 - protein, 250 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 39 - protein, 505 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 40 - protein, 530 aa, Artificial Sequence
  Synthetic Polypeptide
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 41 - protein, 993 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 5 aa, Artificial Sequence
  Synthetic Polypeptide
NYGMH

SEQ ID NO: 43 - protein, 17 aa, Artificial Sequence
  Synthetic Polypeptide
VIWYDGSDKYYADSVRG

SEQ ID NO: 44 - protein, 15 aa, Artificial Sequence
  Synthetic Polypeptide
DGYDILTGNPRDFDY

SEQ ID NO: 45 - protein, 16 aa, Artificial Sequence
  Synthetic Polypeptide
RSSQSLVHSDGNTYLS

SEQ ID NO: 46 - protein, 7 aa, Artificial Sequence
  Synthetic Polypeptide
RISRRFS

SEQ ID NO: 47 - protein, 9 aa, Artificial Sequence
  Synthetic Polypeptide
MQSTHVPRT

SEQ ID NO: 48 - protein, 124 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 49 - protein, 112 aa, Artificial Sequence
  Synthetic Polypeptide
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 50 - protein, 251 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 51 - protein, 506 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 52 - protein, 994 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 5 aa, Artificial Sequence
  Synthetic Polypeptide
DYYMT

SEQ ID NO: 54 - protein, 17 aa, Artificial Sequence
  Synthetic Polypeptide
YISSSGSTIYYADSVKG

SEQ ID NO: 55 - protein, 8 aa, Artificial Sequence
  Synthetic Polypeptide
DRNSHFDY

SEQ ID NO: 56 - protein, 11 aa, Artificial Sequence
  Synthetic Polypeptide
RASQGINTWLA

SEQ ID NO: 57 - protein, 7 aa, Artificial Sequence
  Synthetic Polypeptide
GASGLQS

SEQ ID NO: 58 - protein, 9 aa, Artificial Sequence
  Synthetic Polypeptide
QQAKSFPRT

SEQ ID NO: 59 - protein, 117 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 60 - protein, 107 aa, Artificial Sequence
  Synthetic Polypeptide
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 61 - protein, 239 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 62 - protein, 494 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 63 - protein, 982 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 982 aa, Artificial Sequence
  Synthetic Polypeptide
QVQLVESGGGLVKPGGSLRLSCAASGFTFSDHYMSWIRQAPGKCLEWFSYISSSGGIIYYADSVKGRFTISRDNAKNSLYLQMNSLRAEDTAVYYCARDVGSHFDYWGQGTLVTVSSGGGGSGGGGSGGGGSDIQMTQSPSSVSASVGDRVTITCRASQDISRWLAWYQQKPGKAPKLLISAASRLQSGVPSRFSGSGSGTDFTLTISSLQPEDFAIYYCQQAKSFPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 5 aa, Artificial Sequence
  Synthetic Polypeptide
SYGMH

SEQ ID NO: 66 - protein, 17 aa, Artificial Sequence
  Synthetic Polypeptide
FIWYEGSNKYYAESVKD

SEQ ID NO: 67 - protein, 16 aa, Artificial Sequence
  Synthetic Polypeptide
RAGIIGTIGYYYGMDV

SEQ ID NO: 68 - protein, 11 aa, Artificial Sequence
  Synthetic Polypeptide
SGDRLGEKYTS

SEQ ID NO: 69 - protein, 7 aa, Artificial Sequence
  Synthetic Polypeptide
QDTKRPS

SEQ ID NO: 70 - protein, 9 aa, Artificial Sequence
  Synthetic Polypeptide
QAWESSTVV

SEQ ID NO: 71 - protein, 125 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSS

SEQ ID NO: 72 - protein, 107 aa, Artificial Sequence
  Synthetic Polypeptide
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 73 - protein, 247 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 74 - protein, 507 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 75 - protein, 989 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 987 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 125 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSS

SEQ ID NO: 78 - protein, 106 aa, Artificial Sequence
  Synthetic Polypeptide
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 79 - protein, 246 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 80 - protein, 501 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 81 - protein, 989 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 987 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 7 aa, Artificial Sequence
  Synthetic Polypeptide
NARMGVS

SEQ ID NO: 84 - protein, 16 aa, Artificial Sequence
  Synthetic Polypeptide
HIFSNDEKSYSTSLKN

SEQ ID NO: 85 - protein, 14 aa, Artificial Sequence
  Synthetic Polypeptide
IVGYGSGWYGFFDY

SEQ ID NO: 86 - protein, 11 aa, Artificial Sequence
  Synthetic Polypeptide
RASQGIRNDLG

SEQ ID NO: 87 - protein, 7 aa, Artificial Sequence
  Synthetic Polypeptide
AASTLQS

SEQ ID NO: 88 - protein, 9 aa, Artificial Sequence
  Synthetic Polypeptide
LQHNSYPLT

SEQ ID NO: 89 - protein, 124 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 90 - protein, 107 aa, Artificial Sequence
  Synthetic Polypeptide
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 91 - protein, 246 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 92 - protein, 501 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 93 - protein, 989 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 5 aa, Artificial Sequence
  Synthetic Polypeptide
SYYWS

SEQ ID NO: 95 - protein, 16 aa, Artificial Sequence
  Synthetic Polypeptide
YVYYSGTTNYNPSLKS

SEQ ID NO: 96 - protein, 10 aa, Artificial Sequence
  Synthetic Polypeptide
IAVTGFYFDY

SEQ ID NO: 97 - protein, 12 aa, Artificial Sequence
  Synthetic Polypeptide
RASQRVNNNYLA

SEQ ID NO: 98 - protein, 7 aa, Artificial Sequence
  Synthetic Polypeptide
GASSRAT

SEQ ID NO: 99 - protein, 9 aa, Artificial Sequence
  Synthetic Polypeptide
QQYDRSPLT

SEQ ID NO: 100 - protein, 118 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 101 - protein, 108 aa, Artificial Sequence
  Synthetic Polypeptide
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 102 - protein, 241 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 103 - protein, 496 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 104 - protein, 982 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 5 aa, Artificial Sequence
  Synthetic Polypeptide
SYGMH

SEQ ID NO: 106 - protein, 17 aa, Artificial Sequence
  Synthetic Polypeptide
VISYEGSNKYYAESVKG

SEQ ID NO: 107 - protein, 13 aa, Artificial Sequence
  Synthetic Polypeptide
DRGTIFGNYGLEV

SEQ ID NO: 108 - protein, 122 aa, Artificial Sequence
  Synthetic Polypeptide
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 109 - protein, 16 aa, Artificial Sequence
  Synthetic Polypeptide
RSSQSLLHKNAFNYLD

SEQ ID NO: 110 - protein, 7 aa, Artificial Sequence
  Synthetic Polypeptide
LGSNRAS

SEQ ID NO: 111 - protein, 9 aa, Artificial Sequence
  Synthetic Polypeptide
MQALQTPFT

SEQ ID NO: 112 - protein, 112 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 113 - protein, 525 aa, Artificial Sequence
  Synthetic Polypeptide
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 114 - protein, 1013 aa, Artificial Sequence
  Synthetic Polypeptide
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 5 aa, Artificial Sequence
  Synthetic Polypeptide
SYPIN

SEQ ID NO: 116 - protein, 16 aa, Artificial Sequence
  Synthetic Polypeptide
VIWTGGGTNYASSVKG

SEQ ID NO: 117 - protein, 15 aa, Artificial Sequence
  Synthetic Polypeptide
SRGVYDFDGRGAMDY

SEQ ID NO: 118 - protein, 17 aa, Artificial Sequence
  Synthetic Polypeptide
KSSQSLLYSSNQKNYFA

SEQ ID NO: 119 - protein, 7 aa, Artificial Sequence
  Synthetic Polypeptide
WASTRES

SEQ ID NO: 120 - protein, 9 aa, Artificial Sequence
  Synthetic Polypeptide
QQYYSYPYT

SEQ ID NO: 121 - protein, 123 aa, Artificial Sequence
  Synthetic Polypeptide
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSS

SEQ ID NO: 122 - protein, 113 aa, Artificial Sequence
  Synthetic Polypeptide
DIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 123 - protein, 251 aa, Artificial Sequence
  Synthetic Polypeptide
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 124 - protein, 506 aa, Artificial Sequence
  Synthetic Polypeptide
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 125 - protein, 994 aa, Artificial Sequence
  Synthetic Polypeptide
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 5 aa, Artificial Sequence
  Synthetic Polypeptide
NHIIH

SEQ ID NO: 127 - protein, 17 aa, Artificial Sequence
  Synthetic Polypeptide
YINPYPGYHAYNEKFQG

SEQ ID NO: 128 - protein, 12 aa, Artificial Sequence
  Synthetic Polypeptide
DGYYRDTDVLDY

SEQ ID NO: 129 - protein, 11 aa, Artificial Sequence
  Synthetic Polypeptide
QASQDISNYLN

SEQ ID NO: 130 - protein, 7 aa, Artificial Sequence
  Synthetic Polypeptide
YTSRLHT

SEQ ID NO: 131 - protein, 9 aa, Artificial Sequence
  Synthetic Polypeptide
QQGNTLPWT

SEQ ID NO: 132 - protein, 121 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 133 - protein, 107 aa, Artificial Sequence
  Synthetic Polypeptide
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 134 - protein, 243 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 135 - protein, 498 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 136 - protein, 986 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 5 aa, Artificial Sequence
  Synthetic Polypeptide
DYYMY

SEQ ID NO: 138 - protein, 17 aa, Artificial Sequence
  Synthetic Polypeptide
IISDAGYYTYYSDIIKG

SEQ ID NO: 139 - protein, 12 aa, Artificial Sequence
  Synthetic Polypeptide
GFPLLRHGAMDY

SEQ ID NO: 140 - protein, 11 aa, Artificial Sequence
  Synthetic Polypeptide
KASQNVDANVA

SEQ ID NO: 141 - protein, 7 aa, Artificial Sequence
  Synthetic Polypeptide
SASYVYW

SEQ ID NO: 142 - protein, 9 aa, Artificial Sequence
  Synthetic Polypeptide
QQYDQQLIT

SEQ ID NO: 143 - protein, 121 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 144 - protein, 107 aa, Artificial Sequence
  Synthetic Polypeptide
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 145 - protein, 243 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 146 - protein, 498 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 147 - protein, 986 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 984 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 498 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 150 - protein, 986 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 984 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 5 aa, Artificial Sequence
  Synthetic Polypeptide
DYYMY

SEQ ID NO: 153 - protein, 17 aa, Artificial Sequence
  Synthetic Polypeptide
IISDGGYYTYYSDIIKG

SEQ ID NO: 154 - protein, 12 aa, Artificial Sequence
  Synthetic Polypeptide
GFPLLRHGAMDY

SEQ ID NO: 155 - protein, 11 aa, Artificial Sequence
  Synthetic Polypeptide
KASQNVDTNVA

SEQ ID NO: 156 - protein, 7 aa, Artificial Sequence
  Synthetic Polypeptide
SASYVYW

SEQ ID NO: 157 - protein, 9 aa, Artificial Sequence
  Synthetic Polypeptide
QQYDQQLIT

SEQ ID NO: 158 - protein, 121 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 159 - protein, 107 aa, Artificial Sequence
  Synthetic Polypeptide
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 160 - protein, 243 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 161 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 162 - protein, 986 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 984 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 165 - protein, 986 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 984 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 5 aa, Artificial Sequence
  Synthetic Polypeptide
DYYMY

SEQ ID NO: 168 - protein, 17 aa, Artificial Sequence
  Synthetic Polypeptide
IISDGGYYTYYSDIIKG

SEQ ID NO: 169 - protein, 12 aa, Artificial Sequence
  Synthetic Polypeptide
GFPLLRHGAMDY

SEQ ID NO: 170 - protein, 11 aa, Artificial Sequence
  Synthetic Polypeptide
KASQNVDTNVA

SEQ ID NO: 171 - protein, 7 aa, Artificial Sequence
  Synthetic Polypeptide
SASYVYW

SEQ ID NO: 172 - protein, 9 aa, Artificial Sequence
  Synthetic Polypeptide
QQYDQQLIT

SEQ ID NO: 173 - protein, 121 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSS

SEQ ID NO: 174 - protein, 107 aa, Artificial Sequence
  Synthetic Polypeptide
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 175 - protein, 243 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 176 - protein, 498 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 177 - protein, 986 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 984 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 498 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 180 - protein, 986 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 984 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 10 aa, Artificial Sequence
  Synthetic Polypeptide
DKTHTCPPCP

SEQ ID NO: 183 - protein, 12 aa, Artificial Sequence
  Synthetic Polypeptide
ERKCCVECPPCP

SEQ ID NO: 184 - protein, 16 aa, Artificial Sequence
  Synthetic Polypeptide
ELKTPLDTTHTCPRCP

SEQ ID NO: 185 - protein, 17 aa, Artificial Sequence
  Synthetic Polypeptide
ELKTPLGDTTHTCPRCP

SEQ ID NO: 186 - protein, 12 aa, Artificial Sequence
  Synthetic Polypeptide
ESKYGPPCPSCP

SEQ ID NO: 187 - protein, 5 aa, Artificial Sequence
  Synthetic Polypeptide
GGGGS

SEQ ID NO: 188 - protein, 10 aa, Artificial Sequence
  Synthetic Polypeptide
GGGGSGGGGS

SEQ ID NO: 189 - protein, 15 aa, Artificial Sequence
  Synthetic Polypeptide
GGGGSGGGGSGGGGS

SEQ ID NO: 190 - protein, 20 aa, Artificial Sequence
  Synthetic Polypeptide
GGGGSGGGGSGGGGSGGGGS

SEQ ID NO: 191 - protein, 25 aa, Artificial Sequence
  Synthetic Polypeptide
GGGGSGGGGSGGGGSGGGGSGGGGS

SEQ ID NO: 192 - protein, 30 aa, Artificial Sequence
  Synthetic Polypeptide
GGGGSGGGGSGGGGSGGGGSGGGGSGGGGS

SEQ ID NO: 193 - protein, 35 aa, Artificial Sequence
  Synthetic Polypeptide
GGGGSGGGGSGGGGSGGGGSGGGGSGGGGSGGGGS

SEQ ID NO: 194 - protein, 40 aa, Artificial Sequence
  Synthetic Polypeptide
GGGGSGGGGSGGGGSGGGGSGGGGSGGGGSGGGGSGGGGS

SEQ ID NO: 195 - protein, 6 aa, Artificial Sequence
  Synthetic Polypeptide
PGGGGS

SEQ ID NO: 196 - protein, 6 aa, Artificial Sequence
  Synthetic Polypeptide
PGGDGS

SEQ ID NO: 197 - protein, 6 aa, Artificial Sequence
  Synthetic Polypeptide
SGGGGS

SEQ ID NO: 198 - protein, 4 aa, Artificial Sequence
  Synthetic Polypeptide
GGGG

SEQ ID NO: 199 - protein, 6 aa, Artificial Sequence
  Synthetic Polypeptide
HHHHHH

SEQ ID NO: 200 - protein, 109 aa, Artificial Sequence; identical to SEQ ID NO: 25
  Synthetic Polypeptide

SEQ ID NO: 201 - protein, 125 aa, Artificial Sequence
  Synthetic Polypeptide
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSS

SEQ ID NO: 202 - protein, 249 aa, Artificial Sequence
  Synthetic Polypeptide
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 203 - protein, 227 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 225 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 205 - protein, 227 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 225 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 207 - protein, 227 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 225 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 209 - protein, 227 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 225 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP